(19) United States
(12) Patent Application Publication
(10) Pub. No.: US 2024/0299353 A1
(43) Pub. Date: Sep. 12, 2024

(54) COMPOSITIONS AND METHODS FOR TREATING ADDICTIONS COMPRISING 5-MEO-DMT

(71) Applicants: Alvarius Pharmaceuticals Ltd., Dublin (IE); University College Dublin, National University of Ireland, Dublin, Dublin (IE)

(72) Inventors: Keith J. MURPHY, Dublin (IE); James LINDEN, Dublin (IE)

(21) Appl. No.: 18/550,086

(22) PCT Filed: Mar. 11, 2022
(86) PCT No.: PCT/EP2022/056406

§ 371 Date: Sep. 11, 2023

Related U.S. Application Data
(60) Provisional application No. 63/160,068, filed on Mar. 12, 2021.

(51) Int. Cl. A61K 31/4045; A61K 31/436; A61K 31/438; A61K 31/4535; A61K 31/496; A61K 31/517; A61K 31/519; A61K 31/55; A61P 25/30

(52) U.S. Cl. CPC A61K 31/4045; A61K 31/436; A61K 31/438; A61K 31/4535; A61K 31/496; A61K 31/517; A61K 31/519; A61K 31/55; A61P 25/30

(57) ABSTRACT

Disclosed herein is a method of treating a substance use disorder in a subject suffering from sustained substance exposure, comprising: administering to the subject a therapeutically effective amount of 5-methoxy-N,N-dimethyltryptamine (5-MeO-DMT), or a pharmaceutically acceptable derivative or salt thereof.

### CROSS-REFERENCE TO RELATED APPLICATION

This application claims priority to U.S. Application No. 63/160,068 filed on Mar. 12, 2021, the disclosure of which is incorporated by reference herein in its entirety for all purposes.

### BACKGROUND

Substance addictions manifest an unprecedented medical, financial, and emotional toll on society in the forms of overdose and health complications, family disintegration, loss of employment, and crime. For example, according to a 2018 survey of drug use conducted by the European Monitoring Centre for Drugs and Drug Addiction 5.1% of adults aged 15-64 report cocaine use over their lifetime making it the second most used illicit substance after cannabis in the EU. Of these an estimated 10-20% will develop a cocaine use disorder (CUD).

There currently are no medications approved by the food and drug administration for the treatment of certain stimulant use disorders, including CUD. There is an ongoing unmet need for the investigation and development of new therapeutic agents that may alleviate stimulant (e.g., drug or alcohol) dependency and reduce the long-term risk of relapse that persists after the cessation of stimulant use.

### BRIEF SUMMARY

Disclosed herein are compositions and methods for treating additions. In some cases, disclosed is a method of treating a substance use disorder in a subject suffering from sustained substance exposure, comprising: administering to the subject a therapeutically effective amount of 5-methoxy-N,N-dimethyltryptamine (5-MeO-DMT), or a pharmaceutically acceptable derivative or salt thereof. In some cases, the administering 5-MeO-DMT results in a clinical endpoint for treating the substance use disorder. In some cases, the clinical endpoint comprises reducing self-administration of the substance, decreasing a propensity for relapse, reducing an effect of substance withdrawal, or any combination thereof.

In some cases, disclosed is a method of reducing anxiety or depression of a subject suffering from sustained substance exposure, comprising: administering to the subject a therapeutically effective amount of 5-methoxy-N,N-dimethyltryptamine (5-MeO-DMT), or a pharmaceutically acceptable derivative or salt thereof. In some cases, the method modulates gene expression of a biomarker in the subject.

In some cases, disclosed is a method of modulating gene or protein expression of a biomarker in a subject suffering from sustained substance exposure, comprising: administering to the subject a therapeutically effective amount of 5-methoxy-N,N-dimethyltryptamine (5-MeO-DMT), or a pharmaceutically acceptable derivative or salt thereof.

In some cases, the substance comprises a stimulant or a sedative. In some cases, the stimulant is selected from the group consisting of cocaine, nicotine, methamphetamine, amphetamine, ecstasy, and any combination thereof. In some cases, the sedative is selected from the group consisting of barbiturates, benzodiazepines, antihistamines, antidepressants, opioids, antipsychotics, alcohol, and any combination thereof. In some cases, the sedative is heroin. In some cases, the stimulant is cocaine.

In some cases, the biomarker is measured in a blood or urine sample from the subject. In some cases, the biomarker is selected from the group consisting of FosB, ΔFosB, cAMP response element binding protein (CREB), histone methyltransferase (G9a), histone H3 lysine 9 (H3K9), metabotropic glutamate receptor (mGluR), glucocorticoid receptor (GR), 5-HT_{1A} Receptor (5-HT_{1A}R), and brain-derived neurotrophic factor (BDNF).

In some cases, the biomarker is FosB, and its gene and protein expression is elevated in nucleus accumbens 24 hours after the sustained substance exposure. In some cases, the method reduces the elevated gene or protein expression of FosB in nucleus accumbens. In some cases, the method reduces the elevated gene or protein expression of FosB by about 10% to about 90%. In some cases, the method reduces the elevated gene or protein expression of FosB by at least about 10%. In some cases, the method reduces the elevated gene or protein expression of FosB by at most about 90%. In some cases, the method reduces the elevated gene or protein expression of FosB by about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some cases, the method reduces the elevated gene or protein expression of FosB by about 10%, about 20%, about 30%, about 40%, about 50%, about 60%, about 70%, about 80%, or about 90%.

In some cases, the biomarker is G9a, and its gene or protein expression is elevated in dorsal striatum 24 hours after the sustained substance exposure. In some cases, the method reduces the elevated gene or protein expression of G9a in dorsal striatum. In some cases, the method reduces the elevated gene or protein expression of G9a by about 10% to about 90%. In some cases, the method reduces the elevated gene or protein expression of G9a by at least about 10%. In some cases, the method reduces the elevated gene or protein expression of G9a by at most about 90%. In some cases, the method reduces the elevated gene or protein expression of G9a by about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some cases, the method reduces the elevated gene or protein expression of G9a by about 10%, about 20%, about 30%, about 40% about 50%, about 60%, about 70%, about 80%, or about 90%.

In some cases, the biomarker is ΔFosB, and its gene or protein expression is elevated in dorsal striatum 24 hours after the sustained substance exposure. In some cases, the method reduces the elevated gene or protein expression of ΔFosB in dorsal striatum. In some cases, the method reduces the elevated gene or protein expression of ΔFosB by about 10% to about 90%. In some cases, the method reduces the elevated gene or protein expression of ΔFosB by at least about 10%. In some cases, the method reduces the elevated gene or protein expression of ΔFosB by at most about 90%. In some cases, the method reduces the elevated gene or protein expression of ΔFosB by about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50% about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some cases, the method reduces the elevated gene or protein expression of ΔFosB by about 10%, about 20%, about 30%, about 40%, about 50%, about 60%, about 70%, about 80%, or about 90%.

In some cases, the biomarker is 5-HT_{1A}R, and its gene or protein expression is reduced in hippocampus 24 hours after the sustained substance exposure. In some cases, the method increases the reduced gene or protein expression of 5-HT_{1A}R in hippocampus. In some cases, the method increases the reduced gene or protein expression of 5-HT_{1A}R by about 10% to about 90%. In some cases, the method increases the reduced gene or protein expression of 5-HT_{1A}R by at least about 10%. In some cases, the method increases the reduced gene or protein expression of 5-HT_{1A}R by at most about 90%. In some cases, the method increases the reduced gene or protein expression of 5-HT_{1A}R by about 10% to about 20%, about 10% to about 30%, about 10% to about 40%, about 10% to about 50%, about 10% to about 60%, about 10% to about 70%, about 10% to about 800%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some cases, the method increases the reduced gene or protein expression of 5-HT_{1A}R by about 10%, about 20%, about 30%, about 40%, about 50%, about 60%, about 70%, about 80%, or about 90%.

In some cases, the biomarker is G9a, and its gene or protein expression is reduced in hippocampus 24 hours after the sustained substance exposure. In some cases, the method increases the reduced gene or protein expression of G9a in hippocampus. In some cases, the method increases the reduced gene or protein expression of G9a by about 10% to about 90%. In some cases, the method increases the reduced gene or protein expression of G9a by at least about 10%. In some cases, the method increases the reduced gene or protein expression of G9a by at most about 90%. In some cases, the method increases the reduced gene or protein expression of G9a by about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some cases, the method increases the reduced gene or protein expression of G9a by about 10%, about 20%, about 30% about 40%, about 50%, about 60%, about 70%, about 80%, or about 90%.

In some cases, the biomarker is ΔFosB, and its gene or protein expression is elevated in amygdala 24 hours after the sustained substance exposure. In some cases, the method reduces the elevated gene or protein expression of ΔFosB in amygdala. In some cases, the method reduces the elevated gene or protein expression of ΔFosB by about 10% to about 90%. In some cases, the method reduces the elevated gene or protein expression of ΔFosB by at least about 10%. In some cases, the method reduces the elevated gene or protein expression of ΔFosB by at most about 90%.

In some cases, the method reduces the elevated gene or protein expression of ΔFosB by about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some cases, the method reduces the elevated gene or protein expression of ΔFosB by about 10%, about 20%, about 30%, about 40%, about 50%, about 60%, about 70%, about 80%, or about 90%.

In some cases, the biomarker is 5-HT_{1A}R, and its gene or protein expression is reduced in amygdala 24 hours after the sustained substance exposure. In some cases, the method increases the reduced gene or protein expression of 5-HT_{1A}R in amygdala. In some cases, the method increases the reduced gene or protein expression of 5-HT_{1A}R by about 10% to about 90%. In some cases, the method increases the reduced gene or protein expression of 5-HT_{1A}R by at least about 10%. In some cases, the method increases the reduced gene or protein expression of 5-HT_{1A}R by at most about 90%. In some cases, the method increases the reduced gene or protein expression of 5-HT_{1A}R by about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some cases, the method increases the reduced gene or protein expression of 5-HT_{1A}R by about 10%, about 20% about 30%, about 40%, about 50%, about 60%, about 70%, about 80%, or about 90%.

In some cases, the biomarker is G9a, and its gene or protein expression is reduced in amygdala 24 hours after the sustained substance exposure. In some cases, the method increases the reduced gene or protein expression of G9a in amygdala. In some cases, the method increases the reduced gene or protein expression of G9a by about 10% to about 90%. In some cases, the method increases the reduced gene or protein expression of G9a by at least about 10%. In some cases, the method increases the reduced gene or protein expression of G9a by at most about 90%. In some cases, the method increases the reduced gene or protein expression of G9a by about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some cases, the method increases the reduced gene or protein expression of G9a by about 10%, about 20%, about 30% about 40%, about 50%, about 60%, about 70%, about 80%, or about 90%.

In some cases, the therapeutically effective amount is from about 1 mg/kg to about 50 mg/kg. In some cases, the therapeutically effective amount is from about 5 mg/kg to about 25 mg/kg. In some cases, the therapeutically effective amount is about 10 mg/kg or 20 mg/kg. In some cases, the therapeutically effective amount is about 1 mg/kg to about 50 mg/kg. In some cases, the therapeutically effective amount is at least about 1 mg/kg. In some cases, the therapeutically effective amount is at most about 50 mg/kg. In some cases, the therapeutically effective amount is about 1 mg/kg to about 10 mg/kg, about 1 mg/kg to about 20 mg/kg, about 1 mg/kg to about 30 mg/kg, about 1 mg/kg to about 40 mg/kg, about 1 mg/kg to about 50 mg/kg, about 10 mg/kg to about 20 mg/kg, about 10 mg/kg to about 30 mg/kg, about 10 mg/kg to about 40 mg/kg, about 10 mg/kg to about 50 mg/kg, about 20 mg/kg to about 30 mg/kg, about 20 mg/kg to about 40 mg/kg, about 20 mg/kg to about 50 mg/kg, about 30 mg/kg to about 40 mg/kg, about 30 mg/kg to about 50 mg/kg, or about 40 mg/kg to about 50 mg/kg. In some cases, the therapeutically effective amount is about 1 mg/kg, about 10 mg/kg, about 20 mg/kg, about 30 mg/kg, about 40 mg/kg, or about 50 mg/kg.

In some cases, the method further comprises administering to the subject a therapeutically effective amount of an antidote reversal agent. In some cases, the antidote reversal agent is selected from the group consisting of ketanserin, rapamycin, pizotifen, spiperone, ritanserin, WAY100635, and ANA-12. In some cases, the method further comprises calculating a dose of the antidote reversal agent to be delivered.

In some cases, the 5-MeO-DMT or pharmaceutically acceptable derivative or salt thereof is administered to the subject via a delivery route selected from the group consisting of oral, intravenous, intraperitoneal, intramuscular, intradermal, subcutaneous, intra-arteriole, intraventricular, intracranial, intralesional, intrathecal, topical, transmucosal, intranasal, and a combination thereof.

In some cases, disclosed is a kit, comprising: (a) 5-Methoxy-N,N-dimethyltryptamine (5-MeO-DMT) or pharmaceutically acceptable derivative or salt thereof; and (b) an antidote reversal agent. In some cases, the antidote reversal agent is selected from the group consisting of ketanserin, rapamycin, pizotifen, spiperone, ritanserin, WAY100635, and ANA-12. In some cases, the kit further comprises a digital device configured to calculate a dose of the antidote reversal agent to be delivered. A kit may also comprise instructions for use thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows exemplary serotonin and indolealkylamines chemical structures.

FIG. 2A-2E show exemplary brain regions of interest. Immediately following euthanasia, the rodent whole brain was removed from the skull. The olfactory bulbs highlighted in (FIG. 2A) were removed before the brain was placed in the brain matrix. Blades were inserted in positions 2 and 3 highlighted in (FIG. 2B) to remove a coronal slice 2 mm thick of Bregma coordinates ˜0.2-˜2.2 (FIG. 2C). From this coronal slice a blunted 12 G needle was used to punch dissect the NAc and DS as shown in (FIG. 2D). A diagrammatic representation of the dissected NAc and DS is shown in (FIG. 2E).

FIG. 3A-3D show other exemplary brain regions of interest. Following the removal of the coronal slice for the NAc and DS dissection described in FIG. 2A-2E, the PFC was dissected from the region anterior to the coronal slice (FIG. 3A). The cerebellum was removed from the hind region of the brain, posterior to the slice (˜0.2+Bregma) (FIG. 3B). The hippocampus (FIG. 3C) and the amygdaloid complex (FIG. 3D) were then dissected.

FIG. 4 shows the cocaine±5-MeO-DMT study design in Example 1. Animals received 14 injections of cocaine or saline over 14 days. They were then administered a vehicle, 10 mg/kg 5-MeO-DMT or 20 mg/kg 5-MeO-DMT injection and were euthanized 24 or 120 hours after the last cocaine or saline exposure. Brain regions of interest were dissected, frozen in liquid nitrogen and stored at −80° C. until processing, n=6 per group.

FIG. 5 shows the exemplary antibodies used for western blotting and immunocytochemistry.

FIG. 6A-6C show the exemplary flowchart of hippocampal mass spectrometry. (FIG. 6A) Hippocampal tissue was dissected from the animals in the cocaine+5-MeO-DMT molecular study. Protein was extracted with protein lysis buffer and the samples were prepared for mass spectrometry. Briefly, each sample was denatured, reduced, and alkylated before undergoing trypsinisation overnight to generate short peptide sequences suitable for shotgun proteomics. (FIG. 6B) Samples were loaded into the mass spectrometer, individual peptides were ionized by electrospray ionization and were accelerated through the spectrometer at a flow rate of 250 nl/min. (FIG. 6C) Individual peptides were detected and identified by their mass/charge spectra, this m/z ratio was used to map peptides to their parent protein using the Rat UniProt database and to measure their relative abundance. Between treatment group comparisons were done using the Student's t test (P<0.05) to determine treatment effects on relative protein abundance with n=6 per group.

FIG. 7A-7B show the exemplary 5-HT_{1A}R and 5-HT_{2A}R expression in primary hippocampal neurons. Primary mixed hippocampal cultures were grown for 9 days. Nuclei were stained with DAPI. Neuronal cells in the culture were identified through positive staining for NeuN. 5-HT_{1A}R (FIG. 7A) and 5-HT_{2A}R (FIG. 7B) expression were assessed at 3-5, 7 and 8 DIV.

FIG. 8A-8B show the exemplary timeline of behavioral tests. (FIG. 8A) Cohort 1: animals were administered cocaine daily for 14 days followed by a single administration of 5-MeO-DMT (20 mg/kg) at 1 hr post last cocaine. (FIG. 8B) Cohort 2: animals were administered cocaine daily for 13 days followed by a single administration of 5-MeO-DMT (10 mg/kg) at 24 hr post last cocaine. Coc=cocaine, PPI=pre-pulse inhibition, EPM=elevated plus maze, NO1=1st novel object session, NO2=2nd novel object session, FS=forced swim, FST=forced swim test.

FIG. 9 shows the exemplary open field test. Animals were places in an open field arena and allowed to freely explore the environment for 5 minutes. The number of entries into the centre of the arena, time spent in the centre and time spent in the perimeter of the arena were all measured by an observer blinded to experimental conditions. The red square delineates the region defined as the centre of the arena; the blue area defines the perimeter of the environment. An animal must have all four paws contained within the red square to count as an entry.

FIG. 10A-10B show the exemplary elevated plus maze study. The maze consists of four arms elevated 100 cm above the ground. Each animal was placed in the centre of the maze facing a closed arm and was freely allowed to explore the maze for 5 minutes. The number of entries into and duration of time spent in an open arm of the maze were measured (FIG. 10A). The duration of time spent in a closed arm of the maze was also measured (FIG. 10B). All four of an animal's paws must be on an open arm before it is counted as an entry. The animal in each image is highlighted in a circle.

FIG. 11A-11B show the exemplary Novel Object Recognition study. In the 1st trial (FIG. 11A) two identical objects are placed in the open field arena and animals can freely explore the environment and investigate the objects for 10 minutes. In the 2nd trial (FIG. 11B) animals are returned to the arena 24 hours after the 1st trial and one of the familiar objects has been swapped for a novel object the animal has never encountered. The duration of time the animal interacts with both the novel and the familiar object during the 2nd trial is measured. Interaction with an object is classified as sniffing or touching the object with the animal's snout being orientated toward the object at a distance of <2 cm from the object. Climbing, circling or being adjacent to the object was not be deemed an interaction.

FIG. 12A-12B show the exemplary Forced Swim Test study. The test consists of each animal being placed in a cylinder of water from which it cannot escape. During the pre-test session animals were placed in the cylinder for 15 minutes. After 24 hours animals were returned to the cylinder for a 5-minute test session. For the duration of the test an animals' behavior can be classified as “swimming”, “climbing” or “immobile”. The duration of time spent performing these behaviors was measured by an observer blind to experimental conditions. In (FIG. 12A) the animal highlighted by the circle is classified as “climbing”. In (FIG. 12B) the animal highlighted by the circle is classified as immobile.

FIG. 13A-13C show the effect of chronic cocaine and 5-MeO-DMT on molecular markers in the NAc. Repeated cocaine and a single administration of 5-MeO-DMT alter FosB, ΔFosB and mGluR5 levels in the NAc at 24-hours. (FIG. 13A) There is a significant interaction between cocaine and 5-MeO-DMT (p=0.0110). 20 mg/kg 5-MeO-DMT, cocaine or cocaine+10 mg/kg 5-MeO-DMT increase FosB expression. 20 mg/kg 5-MeO-DMT post cocaine normalizes FosB expression. (FIG. 13B) There is a significant 5-MeO-DMT effect (p=0.0107) and a significant interaction between cocaine and 5-MeO-DMT (p=0.0051). 5-Meo, cocaine and the combination increase ΔFosB expression. (FIG. 13C) There is a significant cocaine effect (p=0.0450) on mGluR5 with no post hoc differences between treatment groups. All values are expressed as mean±SEM, n=6 per group, *=p<0.05 relative to sal+veh as determined by a two-way ANOVA followed by Bonferroni's post hoc test or 1-way ANOVA followed by Dunnett's post hoc test. Sal+Veh (S+V), Sal+10 mgkg 5-MeO-DMT (5+10), Sal+20 mg/kg 5-MeO-DMT (S+20), Cocaine+Veh (C+V), Cocaine+10 mg/kg 5-MeO-DMT (C+10) and Cocaine+20 mg/kg 5-MeO-DMT (C+20).

FIG. 14 shows the summary of cocaine and 5-MeO-DMT induced protein changes in the nucleus accumbens. Summaries of measured protein markers with changes induced by cocaine, 5-MeO-DMT or their combination outlined. -=no change in expression levels, *=significant effect on marker expression with reported p values for each variable determined by ANOVA and ↑=significant increase in marker expression and ↓=significant decrease in marker expression as determined by post hoc testing.

FIG. 15A-15C show the effect of chronic cocaine and 5-MeO-DMT on striatal marker expression. Repeated cocaine and a single administration of 5-MeO-DMT alter FosB, ΔFosB and G9a levels in the striatum at 24 hours (FIG. 15A) There is a significant cocaine effect (p=0.0217) on FosB with no post hoc differences between treatment groups. (FIG. 15B) Cocaine significantly increased ΔFosB expression in the cocaine and cocaine+10 mg/kg 5-MeO-DMT relative to sal+veh. 20 mg/kg 5-MeO-DMT normalises elevated ΔFosB expression post cocaine. (FIG. 15C) There is a significant interaction between cocaine and 5-MeO-DMT (p=0.0010) affecting G9a levels. 20 mg/kg 5-MeO-DMT or repeated cocaine increase G9a in the striatum while 5-MeO-DMT normalised elevated G9a levels in cocaine-experienced animals. All values are expressed as mean±SEM, n=6 per group, *=p<0.05 relative to sal+veh or #=p<0.05 relative to coc+veh as determined by a two-way ANOVA followed by Bonferroni's post hoc test for (FIG. 15A) and (FIG. 15C) or Kruskal-Wallis with Dunn's post hoc test for (FIG. 15B).

FIG. 16 shows the summary of cocaine and 5-MeO-DMT induced protein changes in the dorsal striatum. Summaries of measured protein markers with changes induced by cocaine, 5-MeO-DMT or their combination outlined. -=no change in expression levels, *=significant effect on marker expression with reported p values for each variable determined by ANOVA and ↑=significant increase in marker expression and ↓=significant decrease in marker expression as determined by post hoc testing.

FIG. 17A-17D show the effect of chronic cocaine and 5-MeO-DMT on molecular markers in the PFC. Repeated cocaine and a single administration of 5-MeO-DMT alter ΔFosB, G9a, BDNF and pCREB levels in the PFC at 24-hours. (FIG. 17A) There is a significant cocaine effect (p=0.0411) and a significant interaction between cocaine and 5-MeO-DMT (p=0.0205) with the combination of cocaine and 10 mg/kg 5-MeO-DMT increasing ΔFosB expression. (FIG. 17B) Cocaine and cocaine+20 mg/kg both significantly decreased the expression of G9a relative to the control group. (FIG. 17C) The combination of cocaine and 5-MeO-DMT significantly decreased the expression of BDNF with no post hoc differences between treatment groups at both tested concentrations (FIG. 17D) Cocaine has a significant effect on the expression of pCREB (p=0.0048) with 20 mg/kg 5-MeO-DMT reducing expression in cocaine-experienced animals All values are expressed as mean±SEM, n=6 per group, Φ=p<0.05 relative to sal+10 mg 5-MeO-DMT and ⊖=p<0.05 relative to sal+20 mg/kg 5-MeO-DMT as determined by a two-way ANOVA followed by Bonferroni's post hoc test for (FIG. 17A) and (FIG. 17D), *=p<0.05 relative to sal+veh as determined by Kruskal-Wallis with Dunn's post hoc test for (FIG. 17B) and (FIG. 17C). Sal+Veh (S+V), Sal+10 mgkg 5-MeO-DMT (5+10), Sal+20 mg/kg 5-MeO-DMT (5+20), Cocaine+Veh (C+V), Cocaine+10 mg/kg 5-MeO-DMT (C+10) and Cocaine+20 mg/kg 5-MeO-DMT (C+20).

FIG. 18 shows effect of chronic cocaine and 5-MeO-DMT on mGluR5 in the PFC. Repeated cocaine and a single administration of 5-MeO-DMT alter the levels of mGluR5 in the PFC at 24-hours. 5-MeO-DMT has a significant effect on the expression of mGluR5 in the PFC (p=0.0301) with the 20 mg/kg dose reducing receptor levels. All values are expressed as mean±SEM, n=6 per group, *=p<0.05 relative to sal+veh as determined by a two-way ANOVA followed by Bonferroni's post hoc test. Sal+Veh (S+V), Sal+10 mgkg 5-MeO-DMT (5+10), Sal+20 mg/kg 5-MeO-DMT (5+20), Cocaine+Veh (C+V), Cocaine+10 mg/kg 5-MeO-DMT (C+10) and Cocaine+20 mg/kg 5-MeO-DMT (C+20).

FIG. 19 shows the summary of cocaine and 5-MeO-DMT induced protein changes in the prefrontal cortex. Summaries of measured protein markers with changes induced by cocaine, 5-MeO-DMT or their combination outlined. -=no change in expression levels, *=significant effect on marker expression with reported p values for each variable determined by ANOVA and ↑=significant increase in marker expression and ↓=significant decrease in marker expression as determined by post hoc testing.

FIG. 20A-20C show effect of chronic cocaine and 5-MeO-DMT on molecular marker expression in the amygdala. Repeated cocaine and a single administration of 5-MeO-DMT alter ΔFosB, 5-HT1A and pCREB levels in the amygdala at 24-hours. (FIG. 20A) Cocaine increases ΔFosB expression (p=0.0463) and 20 mg/kg 5-MeO-DMT normalises expression. (FIG. 20B) Cocaine has a significant effect on 5-HT1A (p=0.0172) with no post hoc differences between treatment groups. (FIG. 20C) There is a significant interaction between cocaine and 5-MeO-DMT (p=0.0222) with 20 mg/kg 5-MeO-DMT increasing pCREB in the saline-treated animals. All values are expressed as mean±SEM, n=6 per group, *=p<0.05 and ⊖=p<0.05 relative to sal+veh and sal+20 mg/kg 5-MeO-DMT as determined by a two-way ANOVA followed by Bonferroni's post hoc test. Sal+Veh (S+V), Sal+10 mgkg 5-MeO-DMT (S+10), Sal+20 mg/kg 5-MeO-DMT (S+20), Cocaine+Veh (C+V), Cocaine+10 mg/kg 5-MeO-DMT (C+10) and Cocaine+20 mg/kg 5-MeO-DMT (C+20).

FIG. 21 shows the summary of cocaine and 5-MeO-DMT induced protein changes in the amygdala. Summaries of measured protein markers with changes induced by cocaine, 5-MeO-DMT or their combination outlined. -=no change in expression levels, *=significant effect on marker expression with reported p values for each variable determined by ANOVA and ↑=significant increase in marker expression and ↓=significant decrease in marker expression as determined by post hoc testing.

FIG. 22A-22D show the effect of chronic cocaine and 5-MeO-DMT on hippocampal molecular expression. Repeated cocaine and a single administration of 5-MeO-DMT alter GR, 5-HT1A, G9a and pCREB levels in the hippocampus at 24-hours. (FIG. 22A) Cocaine has a significant effect on the expression of GR (p=0.0434) with no post hoc differences between treatment groups (FIG. 22B) There is a significant interaction between cocaine and 5-MeO-DMT (p=<0.0001). Both 10 mg/kg 5-MeO-DMT and cocaine alone reduce receptor expression, with 20 mg/kg 5-MeO-DMT normalising receptor levels in cocaine experienced animals (FIG. 22C) Cocaine reduces G9a levels, with 20 mg/kg 5-MeO-DMT normalising expression (FIG. 22D) There is a significant interaction between cocaine and 5-MeO-DMT (p=0.0265) with no post hoc differences between treatment groups. All values are expressed as mean±SEM, n=6 per group, *=p<0.01, #=p<0.001 and ⊖=p<0.01 relative to sal+veh, coc+veh and sal+20 mg 5-MeO-DMT respectively, determined by a two-way ANOVA followed by Bonferroni's post hoc test. Sal+Veh (S+V), Sal+10 mgkg 5-MeO-DMT (5+10), Sal+20 mg/kg 5-MeO-DMT (S+20), Cocaine+Veh (C+V), Cocaine+10 mg/kg 5-MeO-DMT (C+10) and Cocaine+20 mg/kg 5-MeO-DMT (C+20).

FIG. 23 shows the summary of cocaine and 5-MeO-DMT induced protein changes in the hippocampus. Summaries of measured protein markers with changes induced by cocaine, 5-MeO-DMT or their combination outlined. -=no change in expression levels, *=significant effect on marker expression with reported p values for each variable determined by ANOVA and ↑=significant increase in marker expression and ↓=significant decrease in marker expression as determined by post hoc testing.

FIG. 24 shows the statistical overrepresentation pathway analysis for differentially expressed proteins in the hippocampus of animals administered 10 mg/kg 5-Meo.

FIG. 25 shows the statistical overrepresentation pathway analysis for differentially expressed proteins in the hippocampus of animals administered 20 mg/kg 5-Meo.

FIG. 26 shows the statistical overrepresentation pathway analysis for differentially expressed proteins in the hippocampus after repeated cocaine exposures.

FIG. 27 shows the statistical overrepresentation pathway analysis for differentially expressed proteins in the hippocampus after cocaine+10 mg/kg 5-MeO-DMT exposures.

FIG. 28 shows the statistical overrepresentation pathway analysis for differentially expressed proteins in the hippocampus after cocaine+20 mg/kg 5-MeO-DMT exposures.

FIG. 29 shows the hierarchical clustering of differentially expressed proteins after cocaine and/or 10 mg/kg 5-MeO-DMT in the hippocampus. Hierarchical clustering was performed on the 340 differentially expressed proteins between the control and cocaine treated animals as determined by an unadjusted Student's t-test, p-value<0.05 with a fold change in expression of ≥±20%. Data was Zscore normalised and clustering based on both rows and columns was carried out using Euclidean distance measures and average linkage. Bright indicates an increase in protein expression while dark represents a decrease in expression. The intensity of the colour corresponds to the distance from the mean Z score of that protein in the control animals. The range of protein expression extends to 3 standards deviations either side of the mean. N=6 per group.

FIG. 30 shows the hierarchical clustering of the mean differentially expressed proteins after cocaine and/or 10 mg/kg 5-MeO-DMT in the hippocampus. Hierarchical clustering was performed on the 340 differentially expressed proteins between the control and cocaine treated animals as determined by an unadjusted Student's t-test, p-value<0.05 with a fold change in expression of ≥±20%. Data was Zscore normalised and clustering based on both rows and columns was carried out using Euclidean distance measures and average linkage. Bright indicates an increase in protein expression while dark represents a decrease in expression. The intensity of the colour corresponds to the distance from the mean Z score of that protein in the control animals. The range of protein expression extends to 3 standards deviations either side of the mean. N=6 per group.

FIG. 31 shows the hierarchical clustering of differentially expressed proteins after cocaine and/or 20 mg/kg 5-MeO-DMT in the hippocampus. Hierarchical clustering was performed on the 340 differentially expressed proteins between the control and cocaine treated animals as determined by an unadjusted Student's t-test, p-value<0.05 with a fold change in expression of ≥±20%. Data was Zscore normalised and clustering based on both rows and columns was carried out using Euclidean distance measures and average linkage. Bright indicates an increase in protein expression while dark represents a decrease in expression. The intensity of the colour corresponds to the distance from the mean Z score of that protein in the control animals. The range of protein expression extends to 3 standards deviations either side of the mean. N=6 per group.

FIG. 32 shows the hierarchical clustering of the mean differentially expressed proteins after cocaine and/or 20 mg/kg 5-MeO-DMT in the hippocampus. Hierarchical clustering was performed on the 340 differentially expressed proteins between the control and cocaine treated animals as determined by an unadjusted Student's t-test, p-value<0.05 with a fold change in expression of ≥±20%. Data was Zscore normalised and clustering based on both rows and columns was carried out using Euclidean distance measures and average linkage. Bright indicates an increase in protein expression while dark represents a decrease in expression. The intensity of the colour corresponds to the distance from the mean Z score of that protein in the control animals. The range of protein expression extends to 3 standards deviations either side of the mean. N=6 per group.

FIG. 33 shows the exemplary patterns in differentially expressed hippocampal proteins after administration of repeated cocaine, 10 mg/kg 5-MeO-DMT or cocaine+10 mg/kg 5-Meo. 340 differentially expressed proteins were identified 24 hours after the last cocaine exposure in a repeated exposure paradigm. 118 differentially expressed proteins were identified 24 hours after administration of 10 mg/kg 5-Meo. 541 differentially expressed proteins were identified in the combination group. Of the 340 differentially expressed proteins in the cocaine group 245 remained in the combination. Of the 118 differentially expressed proteins in the 5-MeO-DMT group 45 were shared with the combination. Differential expression from saline+vehicle control was determined by Student's t-test, unadjusted, p-value<0.05 with a fold change in expression of ≥±20%. N=6 per group.

FIG. 34 shows the exemplary patterns in differentially expressed hippocampal proteins after administration of repeated cocaine, 20 mg/kg 5-MeO-DMT or cocaine+20 mg/kg 5-Meo. 340 differentially expressed proteins were identified 24 hours after the last cocaine exposure in a repeated exposure paradigm. 120 differentially expressed proteins were identified 24 hours after administration of 20 mg/kg 5-Meo. 221 differentially expressed proteins were identified in the combination group. Of the 340 differentially expressed proteins in the cocaine group only 85 remained in the combination. Of the 120 differentially expressed proteins in the 5-MeO-DMT group 57 were shared with the combination. Differential expression from saline+vehicle control was determined by Student's t-test, unadjusted, p-value<0.05 with a fold change in expression of ≥±20%. N=6 per group.

FIG. 35 shows the exemplary patterns in differentially expressed hippocampal proteins 24 hours after acute 5-Meo. 118 differentially expressed proteins were identified in the hippocampus 24 hours after administration of 10 mg/kg 5-Meo. 120 differentially expressed proteins were identified in the hippocampus 24 hours after administration of 20 mg/kg 5-Meo. 32 differentially expressed proteins were shared between the two concentrations of 5-Meo. Differential expression from saline+vehicle control was determined by Student's t-test, unadjusted, p-value<0.05 with a fold change in expression of ≥±20%. N=6 per group.

FIG. 36 shows the exemplary patterns in differentially expressed hippocampal proteins after administration of repeated cocaine, cocaine+10 mg/kg 5-MeO-DMT or cocaine+20 mg/kg 5-Meo. 340 differentially expressed proteins were identified 24 hours after the last cocaine exposure in a repeated exposure paradigm. 544 differentially expressed proteins were identified in the cocaine+10 mg/kg 5-MeO-DMT combination group. 221 differentially expressed proteins were identified in the cocaine+20 mg/kg 5-MeO-DMT combination group. Of the 340 differentially expressed proteins in the cocaine group only 85 remained in the high 5-MeO-DMT combination while 245 were retained in low 5-MeO-DMT combination. 153 differentially expressed proteins were shared between the combination groups. Differential expression from saline+vehicle control was determined by Student's t-test, unadjusted, pvalue<0.05 with a fold change in expression of ≥±20%. N=6 per group.

FIG. 37 shows the exemplary effect of 5-MeO-DMT and cocaine increasing neuronal structural complexity. Cultured hippocampal neurons (3 DIV) were treated with 5-MeO-DMT (20 μM), cocaine (25 μM) or cocaine followed by 5-Meo. Neurons were labelled with NeuN and projections were traced using the Simple Neurite Tracer plugin for Image J. The generated trace images were then subjected to a Sholl analysis to determine structural complexity. Scale bar=5 μM.

FIG. 38A-38D shows the exemplary effect of 5-MeO-DMT and cocaine increasing neuronal structural complexity. Cultured hippocampal neurons (3 DIV) were treated with 5-MeO-DMT (20 μM), cocaine (25 μM) or cocaine followed by 5-Meo. Neurons were labelled with NeuN and neurites traced using the Simple Neurite Tracer followed by a Sholl analysis using plugins in image J. (FIG. 38A) Sholl analysis generated a Sholl plot showing the number of neurites at a given distance from the centre of the cell soma. (FIG. 38B) Normalised Area under the curve (AUC) of the Sholl plots in (FIG. 38A) demonstrates that both 5-MeO-DMT and cocaine alone or in combination increase dendritic arbor complexity. (FIG. 38C) Maximum number of crossings of the Sholl plots in (FIG. 38A). (FIG. 38D) Average neurite length of Sholl plots in (FIG. 38A). (FIG. 38A) is Mean and (FIG. 38B-38D) is Mean±SEM. N=3, n=169-180 per group. Data was analysed by a one-way ANOVA followed by Bonferroni's post hoc test. *=p<0.05 relative to control, #=p<0.05 relative to cocaine and ⊖=p<0.05 relative to 5-Meo.

FIG. 39 shows the exemplary effect of the 5-HT_{1A}R antagonist WAY100635 preventing 5-Meo-DMT and cocaine-induced increases in neuronal structural complexity. Cultured hippocampal neurons pre-incubated with WAY100635 (100 nM) or vehicle prior to treatment with 5-MeO-DMT (20 μM), cocaine (25 μM) or cocaine followed by 5-Meo. Neurons were labelled with NeuN and projections were traced using the Simple Neurite Tracer plugin for Image J. The generated trace images were then subjected to a Sholl analysis to determine structural complexity. Scale bar=5 μM.

FIG. 40A-40D show the exemplary effect of 5-HT_{1A}R antagonist WAY100635 preventing 5-Meo-DMT and cocaine-induced increases in neuronal structural complexity. Cultured hippocampal neurons (3 DIV) were pre-treated with WAY100635 (100 nM) or vehicle for 15 minutes prior to treatment with 5-MeO-DMT (20 μM), cocaine (25 μM) or cocaine followed by 5-Meo. (FIG. 40A) Sholl plot showing the number of neurites at a given distance from the centre of the cell soma. (FIG. 40B) Normalised AUC of the Sholl plots in (FIG. 40A). WAY100635 prevented both the 5-MeO-DMT and cocaine-induced increase in AUC. (FIG. 40C) Maximum number of crossings of the Sholl plots in (FIG. 40A). WAY100635 decreased Nmax in all treatment groups. (FIG. 40D) Average neurite length of Sholl plots in (FIG. 40A). WAY100635 prevented the 5-Meo-mediated increase in neurite length. (FIG. 40A) is Mean and (FIG. 40B-40D) is Mean±SEM. N=3 and n=110-180 per group. Data was analysed by a oneway ANOVA followed by Bonferroni's post hoc test. *=p<0.05 relative to control, #=p<0.05 relative to cocaine ⊖=p<0.05 relative to 5-MeO-DMT and Φ=p<0.05 relative to cocaine+5-Meo.

FIG. 41 shows the exemplary effect of the 5-HT_{2A}R antagonist Ketanserin reducing neuronal structural complexity. Cultured hippocampal neurons (3 DIV) were pre-incubated with ketanserin (100 μM) or vehicle prior to treatment with 5-MeO-DMT (20 μM), cocaine (25 μM) or cocaine followed by 5-Meo. Neurons were labelled with NeuN and projections were traced using the Simple Neurite Tracer plugin for Image J. The generated trace images were then subjected to a Sholl analysis to determine structural complexity. Scale bar=50 WM.

FIG. 42A-42D show the exemplary effect of the 5-HT_{2A}R antagonist Ketanserin reducing neuronal structural complexity. Cultured hippocampal neurons were pre-treated with Ketanserin (100 μM) or DMSO vehicle for 15 minutes prior to treatment with 5-MeO-DMT (20 μM) cocaine (25 μM) or cocaine followed by 5-Meo. (FIG. 42A) Sholl plot showing the number of neurites at a given distance from the centre of the cell soma. (FIG. 42B) Normalised AUC of the Sholl plots in (FIG. 42A). Ketanserin decreased the AUC across all groups. Cocaine+5-MeO-DMT reduced AUC relative to control (FIG. 42C) Maximum number of crossings of the Sholl plots in (FIG. 42A). Ketanserin decreased Nmax in all treatment groups. (FIG. 42D) Average neurite length of Sholl plots in (FIG. 42A). Ketanserin decreased neurite length in all groups. (FIG. 42A) is Mean and (FIG. 42B-42D) is Mean±SEM. N=3 and n=130-150 per group. Data was analysed by a one-way ANOVA followed by Bonferroni's post hoc test. *=p<0.05 relative to control, #=p<0.05 relative to cocaine ⊖=p<0.05 relative to 5-MeO-DMT and Φ=p<0.05 relative to cocaine+5-Meo.

FIG. 43 shows the exemplary effect of ANA-12 preventing 5-Meo- and cocaine-induced increases in neuronal structural complexity. Cultured hippocampal neurons (3 DIV) were pre-incubated with ANA-12 (10 μM) or vehicle prior to treatment with 5-MeO-DMT (20 μM), cocaine (25 μM) or cocaine followed by 5-Meo. Neurons were labelled with NeuN and projections were traced using the Simple Neurite Tracer plugin for Image J. The generated trace images were then subjected to a Sholl analysis to determine structural complexity. Scale bar=5 μM.

FIG. 44A-44D show the exemplary effect of ANA-12 preventing 5-Meo- and cocaine-induced increases in neuronal structural complexity. Cultured hippocampal neurons were pre-treated with ANA-12 (10 μM) or DMSO vehicle for 15 minutes prior to treatment with 5-MeO-DMT (20 μM) cocaine (25 μM) or cocaine followed by 5-Meo. (FIG. 44A) Sholl plot showing the number of neurites at a given distance from the centre of the cell soma. (FIG. 44B) Normalised AUC of the Sholl plots in (FIG. 44A). ANA-12 prevented the increase in AUC mediated by 5-MeO-DMT or cocaine alone or in combination. (FIG. 44C) Maximum number of crossings of the Sholl plots in (FIG. 44A). ANA-12 pre-treatment decreased Nmax in the cocaine+5-MeO-DMT combination. (FIG. 44D) Average neurite length of Sholl plots in (FIG. 44A). 5-MeO-DMT alone or in combination with cocaine increased the average neurite length. ANA-12 pre-treatment inhibited this increase. (FIG. 44A) is Mean and (FIG. 44B-44D) is Mean±SEM. N=3 and n=129-180 per group. Data was analysed by a one-way ANOVA followed by Bonferroni's post hoc test. *=p<0.05 relative to control, #=p<0.05 relative to cocaine ⊖=p<0.05 relative to 5-MeO-DMT and Φ=p<0.05 relative to cocaine+5-Meo.

FIG. 45 shows the exemplary effect of the rapamycin preventing 5-Meo- and cocaine-induced increases in neuronal structural complexity. Cultured hippocampal neurons (3 DIV) were pre-incubated with rapamycin (100 nM) or vehicle prior to treatment with 5-MeO-DMT (20 μM), cocaine (25 μM) or cocaine followed by 5-Meo. Neurons were labelled with NeuN and projections were traced using the Simple Neurite Tracer plugin for Image J. The generated trace images were then subjected to a Sholl analysis to determine structural complexity. Scale bar=5 μM.

FIG. 46A-46D show the exemplary effect of rapamycin preventing 5-Meo- and cocaine-induced increases in neuronal structural complexity. Cultured hippocampal neurons were pre-treated with rapamycin (100 nM) or DMSO vehicle for 15 minutes prior to treatment with 5-MeO-DMT (20 μM), cocaine (25 μM) or cocaine followed by 5-Meo. (FIG. 46A) Sholl plot showing the number of neurites at a given distance from the centre of the cell soma. (FIG. 46B) Normalised AUC of the Sholl plots in (A). Rapamycin prevented the increase in AUC mediated by 5-MeO-DMT or cocaine alone and decreased the AUC in combination. (FIG. 46C) Maximum number of crossings of the Sholl plots in (FIG. 46A). (FIG. 46D) Average neurite length of Sholl plots in (FIG. 46A). (FIG. 46A) is Mean and (FIG. 46B-46D) is Mean±SEM. N=3 and n=90-180 per group. Data was analysed by a one-way ANOVA followed by Bonferroni's post hoc test. *=p<0.05 relative to control, #=p<0.05 relative to cocaine ⊖=p<0.05 relative to 5-MeO-DMT and D=p<0.05 relative to cocaine+5-Meo.

FIG. 47A-47D show the exemplary meta-Analysis of neuronal structure antagonist studies. Cultured hippocampal neurons were pre-treated with an antagonist or vehicle control for 15 minutes prior to treatment with 5-MeO-DMT (20 μM), cocaine (25 μM) or cocaine followed by 5-Meo. Sholl plots measuring the number of neurites at a given distance from the centre of the cell soma were used to generate AUCs for each treatment condition. To account for variability in each culture the vehicle pre-treated groups were combined to strengthen observations in each of the antagonist experiments. The normalised AUC for the WAY100635 (FIG. 47A), Ketanserin (FIG. 47B), ANA-12 (FIG. 47C) and Rapamycin (FIG. 47D) studies are shown. 5-MeO-DMT and cocaine alone or in combination increase neuronal structural complexity in the vehicle pretreated groups (FIG. 47A-47D). WAY100635 specifically prevents the cocaine-induced increase in complexity (A). Ketanserin prevents the treatment induced increase in complexity and reduces neuronal complexity across all treatment groups relative to control (FIG. 47B). ANA-12 prevents the increase in structural complexity mediated by any of the treatments without affecting basal complexity (FIG. 47C). Rapamycin pre-treatment prevents the cocaine and combination-induced increase in complexity (FIG. 47D). Data is mean±SEM. N=3-15 and n=90-810 per group. Data was analysed by a one-way ANOVA followed by Bonferroni's post hoc test. *=p<0.05 relative to control, #=p<0.05 relative to cocaine ⊖=p<0.05 relative to 5-MeO-DMT and D=p<0.05 relative to cocaine+5-Meo.

FIG. 48A-48D show the Open Field cohort 1 study. Animals were administered cocaine daily for 14 days followed by a single administration of 5-MeO-DMT at 1 hr post last cocaine. 24 hours later they underwent the open field test. The number of entries into the centre of the chamber (FIG. 48A), the duration of time spent in the centre of the chamber (FIG. 48B), the number of vertical movements i.e. rearing (FIG. 48C) and the total time spent rearing (FIG. 48D) were quantified. There was a significant effect of 5-MeO-DMT on entries into the centre of the maze (p=0.0139) albeit with no differences between specific treatment groups. 5-MeO-DMT combined with cocaine decreased the number of rearings relative to sal+veh and coc+veh. The cocaine and 5-MeO-DMT combination also decreased the duration of rearings relative to sal+veh. All graphs are mean±SEM, 3≤n≤8 per group. *=p<0.05 relative to saline+vehicle, #=p<0.05 relative to cocaine+veh.

FIG. 49A-49B show the exemplary Open Field movement study. The total distance travelled by each animal over the duration of the test was measured for cohort 1 (FIG. 49A) and cohort 2 (FIG. 49B). 5-MeO-DMT had a significant effect on the distance travelled in both cohort 1 (p=0.003) and cohort 2 (p=0.0098). Cocaine had a significant effect on distance travelled in cohort 2 (p=0.0129) with no post hoc differences between treatment groups. 5-MeO-DMT significantly reduced the distance travelled in the saline-experienced animals in both cohorts relative to the control animals. 20 mg/kg 5-MeO-DMT combined with cocaine decreased the distance travelled relative to the coc+veh group. All graphs are mean±SEM, 3≤n≤8 per group. *=p<0.05 relative to saline+vehicle, #=p<0.05 relative to cocaine+veh.

FIG. 50A-50D show the exemplary Open Field cohort 2 study. Animals were administered cocaine daily for 13 days followed by a single administration of 5-MeO-DMT 24 hrs post last cocaine. 24 hours later they underwent the open field test. The number of entries into the centre of the chamber (FIG. 50A), the duration of time spent in the centre of the chamber (FIG. 50B), the number of vertical movements i.e. rearing (FIG. 50C) and the total time spent rearing (FIG. 50D) were quantified. Cocaine affected the entries into the centre of the maze (p=0.0448) and the time spent in the centre of the maze (p=0.0429), with a significant increase in the time in the centre of the maze relative to the saline+vehicle group. There was a significant effect of 5-MeO-DMT on the number (p=0.0031) but not the duration of rearings (p=0.0837) as determined by two-way ANOVA with no differences between specific treatment groups found by Bonferroni post hoc testing. All graphs are mean±SEM, 6≤n≤8 per group. *=p<0.05 relative to saline+vehicle.

FIG. 51 shows the exemplary pre-pulse inhibition cohort 1. The percentage inhibition of the basal startle response to a startle tone preceded by a pre-pulse of varying dB intensity are quantified. The dB intensity of the pre-pulse significantly affected the inhibition of the startle response (p=0.0002). The % inhibition of startle in the combination groups was significantly reduced relative to sal+veh and sal+20 mg/kg 5-MeO-DMT at 80 dB. All graphs are mean±SEM, 3≤n≤7 per group. *=p<0.05 relative to sal+veh ⊖=p<0.05 relative to sal+20 mg/kg 5-Meo.

FIG. 52 shows the exemplary pre-pulse inhibition cohort 2. The percentage inhibition of the basal startle response to a startle tone preceded by a pre-pulse of varying dB intensity are quantified. The decibel intensity of the pre-pulse significantly affected the inhibition of the startle response (p=0.0011) there were no treatment specific effects detected. Φ=p<0.05 relative to coc+10 mg/kg 5-MeO-DMT All graphs are mean±SEM, 4≤n≤7 per group.

FIG. 53A-53F show the exemplary Elevated Plus Maze cohort 1 and 2. Cohort 1 (FIGS. 53A, 53C and 53E) and cohort 2 (FIGS. 53B, 53D and 53F). The entries into the open arms (FIG. 53A) and (FIG. 53B), and the duration of time (FIG. 53C) and (FIG. 53D) spent on the open arms of the maze were quantified. There were no effects of cocaine or 5-MeO-DMT on either measure in cohort 1. In cohort 2 cocaine affected both the number of entries (p=0.0055) and the duration of time (p=0.0031) on the open arms of the maze. Time on the open arm of the maze was increased in the combination group relative to control and 5-MeO-DMT alone. The total distance travelled was affected by cocaine in cohort 1 (FIG. 53E) but not cohort 2 (FIG. 53F) with a decrease in the combination group relative to the control and cocaine groups. All graphs are mean±SEM, 3≤n≤8 per group. *=p<0.05 relative to saline+vehicle ⊖=p<0.05 relative to saline+5-MeO-DMT #=p<0.05 relative to cocaine+vehicle.

FIG. 54A-54D show the exemplary traces from Elevated Plus Maze Cohort 1. Representative traces from animals in the saline+vehicle (FIG. 54A), saline+20 mg/kg 5-MeO-DMT (FIG. 54B), cocaine+vehicle (FIG. 54C) and (FIG. 54D) cocaine+20 mg/kg 5-MeO-DMT groups. 3≤n≤8 per group.

FIG. 55A-55D show the exemplary traces from Elevated Plus Maze Cohort 2. Representative traces from animals in the saline+vehicle (FIG. 55A), saline+10 mg/kg 5-MeO-DMT (FIG. 55B), cocaine+vehicle (FIG. 55C) and (FIG. 55D) cocaine+10 mg/kg 5-MeO-DMT groups. 6≤n≤8 per group.

FIG. 56A-56B show the exemplary Elevated Plus Maze controls for cohort 1 and 2. The control groups from cohort 1 and cohort 2 were compared for their number of entries (FIG. 56A) and time on open arms (FIG. 56B) of the EPM. In both parameters there was a significant decrease in the control animals of cohort 2 as determined by a one-tailed t-test. All graphs are mean±SEM, n=8 per group. *=p<0.05 relative to saline+vehicle 1.

FIG. 57 shows the exemplary entries into open arms and duration on open arms of the EPM for control animals in cohort 1 and 2.

FIG. 58 shows the exemplary Novel Object cohort 1. Animals in cohort 1 underwent the second session of novel object testing at 72 hours post last cocaine. The time interacting with both the novel and familiar objects in the open field arena were quantified over a 5-minute test. There were no treatment effects detected. There was no bias towards interaction with the novel over the familiar object. All graphs are mean±SEM, 3≤n≤8 per group.

FIG. 59 shows the exemplary Novel Object cohort 2. Animals in cohort 2 underwent the second session of novel object testing at 96 hours post last cocaine. The time interacting with both the novel and familiar objects in the open field arena were quantified over a 5-minute test. There were no treatment effects detected. There was no bias towards interaction with the novel over the familiar object. All graphs are mean±SEM, 6≤n≤8 per group.

FIG. 60 shows the exemplary Forced Swim Test cohort 1. Animals in cohort 1 underwent the forced swim test at 96 hours post last cocaine. The duration of time spent immobile, swimming or climbing were quantified over the 5-minute test period. All graphs are mean±SEM, 3≤n≤8 per group.

FIG. 61 shows the exemplary Forced Swim Test cohort 2. Animals in cohort 2 underwent the forced swim test at 120 hours post last cocaine. The duration of time spent immobile, swimming or climbing were quantified over the 5-minute test period. There was an increase in climbing in the combination group relative to the control animals. All graphs are mean±SEM, 6≤n≤8 per group. *=p<0.05 relative to saline+vehicle.

FIG. 62 is an exemplary graphic showing long term transcriptional changes in brain regions believed to cause adaptations in neural and behavioural plasticity which may present clinically as a 3-stage addiction cycle. Initial effects of all drugs of abuse enhance the activity of neurons with cell bodies in the VTA that project to the NAc (green arrow). The transcription factor ΔFosB has been identified as a potential “molecular switch” that bridges the gap between casual and compulsive drug use which presents behaviourally as an addiction cycle involving 3 stages. VTA=ventral tegmental area, NAC nucleus accumbens, STR=striatum, HIP=hippocampus, PFC=pre-frontal cortex, AMY=amygdala.

FIG. 63A-FIG. 63E show results of a comparison of the differential proteomic profile of 5-MeO, LSD, Psilocybin or DOI treatment in the hippocampus of chronic cocaine treated animals. FIG. 63A is an exemplary In vivo study design. A single administration of either 5-MeO-DMT, LSD, psilocybin or DOI alters the proteomic profile of the hippocampus after repeated cocaine administration. FIG. 63B-FIG. 63E are Venn diagrams indicating the overlap in the differential proteomes by pairwise comparisons with the saline control group in the hippocampus. Data are the number of proteins significantly regulated (FC: +/−20%, p<0.05, Student's t-test) in each pairwise comparison.

FIG. 64A-FIG. 64B shows results of a comparison of the differential proteomic profile of 5-MeO, LSD, Psilocybin or DOI treatment in the hippocampus of chronic cocaine treated animals. FIG. 64A is a STRING network of all proteins found to be significantly regulated (p<0.05) in the hippocampus of saline v cocaine or saline v cocaine+5-MeO-DMT or saline v cocaine+LSD or saline v cocaine+psilocybin or saline v cocaine+DOI animals. Signed fold change difference is visualized as split donut charts around node. FIG. 64B is a STRING network of the 85 significantly regulated proteins between saline and cocaine (p<0.05, FC: +1.2). Signed fold change difference of saline v cocaine was mapped to nodes using a blue to red gradient. Signed fold change difference of saline v cocaine+psychedelic is visualized as split donut charts around node.

FIG. 65A-FIG. 65B shows results of a comparison of the differential proteomic profile of 5-MeO, LSD, Psilocybin or DOI treatment in the hippocampus of chronic cocaine treated animals. FIG. 65A is a comparison of the magnitude of protein changes mediated by a single dose of each of the psychedelics in the control hippocampus and the hippocampus following chronic cocaine administration. Data are the number of proteins significantly regulated (p<0.05, FC: +/−20%, Student's test versus the saline-saline control group or cocaine-saline group) by psychedelic treatment. FIG. 65B is a comparison of the magnitude of reversal of cocaine-mediated protein expression changes in the hippocampus following treatment with each psychedelic. Data are expressed as the percentage of proteins reversed to control and remaining altered by cocaine following a single treatment with each psychedelic. Data are the number of proteins significantly regulated (p<0.05, FC: +/−20%, Student's t-test) in each pairwise comparison.

FIG. 66 shows pathway analysis of molecular reversal of cocaine-mediated change in biological signalling. This list represents the top 10 biological pathways identified to be dysregulated at a transcriptional level in the hippocampus by chronic cocaine. The correction of each pathway by either 5-MeO-DMT, LSD or psilocybin is indicated by a green box with Yes while pathways that remain dysregulated following psychedelic treatment are indicated by a red box with the word No.

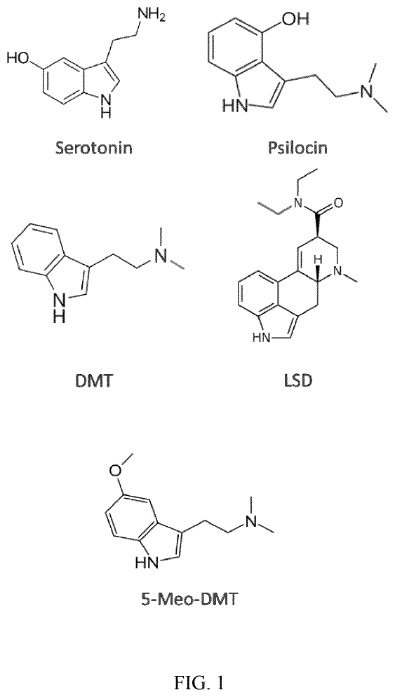

### DETAILED DESCRIPTION

It is to be understood that unless otherwise indicated the present disclosure is not limited to specific formulation components, drug delivery systems, manufacturing techniques, administration steps, or the like, as such may vary. In this regard, unless otherwise stated, a reference to a compound or component includes the compound or component by itself, as well as the compound or component in combination with other compounds or components, such as mixtures of compounds.

### Definitions

As used herein, the singular forms “a,” “an,” and “the” include plural referents unless the context clearly dictates otherwise. Thus, for example, reference to “a pharmaceutical agent” includes not only a single active agent but also a combination or mixture of two or more different active agents.

Reference herein to “one embodiment,” “one version,” or “one aspect” shall include one or more such embodiments, versions or aspects, unless otherwise clear from the context.

As used herein, the term “salt” is intended to include, but not be limited to, pharmaceutically acceptable salts.

As used herein, the term “pharmaceutically acceptable salt” is intended to mean those salts that retain one or more of the biological activities and properties of the free acids and bases and that are not biologically or otherwise undesirable. Illustrative examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, di nitrobenzoates, hydroxybenzoates, methoxybenzoates, phthalates, sulfonates, xylenesulfonates, phenylacetates, phenyipropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

If the pharmaceutical agent is a base, the desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acids such as glucuronic acid and galacturonic acid, alpha-hydroxy acids such as citric acid and tartaric acid, amino acids such as aspartic acid and glutamic acid, aromatic acids such as benzoic acid and cinnamic acid, sulfonic acids such as p-toluenesulfonic acid and ethanesulfonic acid, or the like.

If the pharmaceutical agent is an acid, the desired salt may be prepared by any suitable method known in the art, including treatment of the free acid with an inorganic or organic base, such as an amine (primary, secondary or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines such as piperidine, morpholine and piperazine, and inorganic salts derived from sodium, calcium, potassium, magnesium, manganese, iron, copper, zinc, aluminum, and lithium.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

As used herein, the terms “treating” and “treatment” can refer to reduction in severity and/or frequency of symptoms, elimination of symptoms and/or underlying cause, reduction in likelihood of the occurrence of symptoms and/or underlying cause, and/or remediation of damage. Thus, “treating” a patient with a pharmaceutical agent as provided herein includes prevention of a particular condition, disease, or disorder in a susceptible individual as well as treatment of a clinically symptomatic individual.

As used herein, “effective amount” can refer to an amount covering both therapeutically effective amounts and prophylactically effective amounts.

As used herein, a “therapeutically effective amount” of an active agent refers to an amount that is effective to achieve a desired therapeutic result. A therapeutically effective amount of a given active agent will typically vary with respect to factors such as the type and severity of the disorder or disease being treated and the age, gender, and weight of the patient. In some cases, “inhalation” can refer to inhalation delivery of a therapeutically effective amount of a pharmaceutical agent contained in one unit dose receptacle, which, in some instance, can require one or more breaths, like 1, 2, 3, 4, 5, 6, 7, 8, 9, or more breaths. For example, if the effective amount is 90 mg, and each unit dose receptacle contains 30 mg, the delivery of the effective amount can require 3 inhalations. Unless otherwise specified, the term “therapeutically effective amount” can include a “prophylactically effective amount,” e.g., an amount of active agent that is effective to prevent the onset or recurrence of a particular condition, disease, or disorder in a susceptible individual.

As used herein, “passive dry powder inhaler” can refer to an inhalation device that relies upon a patient's inspiratory effort to disperse and aerosolize a pharmaceutical composition contained within the device in a reservoir or in a unit dose form and does not include inhaler devices which comprise a means for providing energy, such as pressurized gas and vibrating or rotating elements, to disperse and aerosolize the drug composition.

As used herein, “active dry powder inhaler” can refer to an inhalation device that does not rely solely on a patient's inspiratory effort to disperse and aerosolize a pharmaceutical composition contained within the device in a reservoir or in a unit dose form and does include inhaler devices that comprise a means for providing energy to disperse and aerosolize the drug composition, such as pressurized gas and vibrating or rotating elements.

By a “pharmaceutically acceptable” component is meant a component that is not biologically or otherwise undesirable, e.g., the component may be incorporated into a pharmaceutical formulation of the disclosure and administered to a patient as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “derivative” can be used interchangeably with the term “analog.” Compound A can be a derivative or analog of compound B if 1, 2, 3, 4, or 5 atoms of compound A is replaced by another atom or a functional group (e.g., amino, halo, substituted or unsubstituted alkyl, substituted or unsubstituted aryl, substituted or unsubstituted arylalkyl, or substituted or unsubstituted cycloalkyl) to form compound B.

### Composition

The pharmaceutical composition may comprise 5-Methoxy-N,N-dimethyltryptamine (5-MeO-DMT or 5-Meo), or any pharmaceutically acceptable salt or derivative thereof 5-MeO-DMT is a naturally-occurring psychedelic substance of the tryptamine class and can be found in a wide variety of plant species, as well as in the venom of a psychoactive toad species (Bufo Alvarius). 5-MeO-DMT is thought to bind to serotonin receptors in the brain but little is known regarding its neurobiological mechanisms. Exemplary methods of using 5-MeO-DMT are disclosed in WO2020169851.

5-MeO-DMT is a potent, fast-acting hallucinogen with a short duration of action. It induces various physiological and behavioural changes in animal models and it is 4- to 10-fold more potent than DMT in human subjects (Fantegrossi, W. E., K. S. Murnane & C. J. Reissig (2008) The behavioral pharmacology of hallucinogens. Biochemical pharmacology, 75, 17-33; Duvvuri, V., V. B. Risbrough, W. H. Kaye & M. A. Geyer (2009) 5-HT1A receptor activation is necessary for 5-MeODMT-dependent potentiation of feeding inhibition. Pharmacology Biochemistry and Behavior, 93, 349-353; McKenna Dj Fau & Towers, G. H. (1984) Biochemistry and pharmacology of tryptamines and beta-carbolines. A minireview. Journal of Psychoactive Drugs, 16(4), pp. 347-358.)

In humans, 5-MeO-DMT causes distorted perception of time, and changes in processing of visionary and auditory stimuli. Following insufflation the effects start at 3-4 min, peak about 35-40 min, and end around 60-70 min (Ott 2001). It is metabolized through two primary pathways, deamination by MAO-A and O-demethylation by cytochrome P450 2D6 (CYP2D6) to produce an active metabolite, bufotenine (5-hydroxy,N,Ndimethyltryptamine). 5-MeO-DMT is a non-selective 5-HT receptor agonist acting at 5-HT_{1A}, 5-HT_{2A} and 5-HT_{2C} receptors. When canonical signaling activity was measured after administration its highest affinity was for the 5-HT_{1A}R subtype with much higher affinity (Ki, <10 nM) than the 5-HT₂Rs (>1000 nM) (Spencer, D. G., Jr., J. Glaser T Fau—Traber & J. Traber (1987) Serotonin receptor subtype mediation of the interoceptive discriminative stimuli induced by 5-methoxy-N,N-dimethyltryptamine. Psychopharmacology, 93(2), pp. 158-166). However, when non-canonical signaling was measured by assessing liberation of arachidonic acid its affinity for the 5-HT_{2A}R was substantially higher with a reported Ki of 190 nM (Kurrasch-Orbaugh, D. M., V. J. Watts, E. L. Barker & D. E. Nichols (2003b) Serotonin 5-Hydroxytryptamine 2A Receptor-Coupled Phospholipase C and Phospholipase A2 Signaling Pathways Have Different Receptor Reserves. Journal of Pharmacology and Experimental Therapeutics, 304, 229). The apparent non-selectivity of 5-MeO-DMT may be attributed, at least in part, to bufotenine produced from drug metabolism which has a much higher affinity for the 5-HT_{2A}R than the parent compound (Shen et al. 2010). After i.p. administration 5-MeO-DMT reaches maximal drug concentration at 5-7 min and is eliminated with a terminal half-life (t½) of 12-19 min in rodents (Sitaram, B. R., L. Lockett, R. Talomsin, G. L. Blackman & W. R. McLeod (1987) In vivo metabolism of 5-methoxy-N,N-dimethyltryptamine and N,N-dimethyltryptamine in the rat. Biochemical Pharmacology, 36, 1509-1512). 5-MeO-DMT readily crosses the blood-brain barrier and is widely distributed in different rat brain regions including the cortex, thalamus, hippocampus, basal ganglia, medulla, pons and cerebellum. Drug concentrations are increased non-proportionally throughout the brain with an increase in dose administered (Shen, S., X. Jiang, J. Li, R. M. Straubinger, M. Suarez, C. Tu, X. Duan, A. C. Thompson & J. Qu (2016) Large-Scale, Ion-Current-Based Proteomic Investigation of the Rat Striatal Proteome in a Model of Short- and Long-Term Cocaine Withdrawal. Journal of Proteome Research, 15, 1702-1716). Drug toxicology assessments have been performed primarily using an acute administration with little data available on the effects of repeat drug exposures. Acute 5-MeO-DMT in animals can dose-dependently produce ataxia, mydriasis, head nodding, tremor, convulsion and shivering (Gillin et al. 1976). The LD50 of 5-MeO-DMT ranges from 48 to 278 mg/kg for different routes of administration in mice. There is limited toxicity data available for 5-MeO-DMT use in humans. 5-MeO-DMT may offer an alternative to other psychedelic agents for the treatment of mood disorders with possibly superior therapeutic efficacy due to its unique serotonergic pharmacology.

### Formulation

The pharmaceutical composition may include one or more pharmaceutically acceptable excipient. Examples of pharmaceutically acceptable excipients include, but are not limited to, lipids, metal ions, surfactants, amino acids, carbohydrates, buffers, salts, polymers, and the like, and combinations thereof.

Examples of lipids include, but are not limited to, phospholipids, glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate.

Examples of metal ions include, but are not limited to, divalent cations, including calcium, magnesium, zinc, iron, and the like. For instance, when phospholipids are used, the pharmaceutical composition may also comprise a polyvalent cation, as disclosed in WO 01/85136 and WO 01/85137, which are incorporated herein by reference in their entireties. The polyvalent cation may be present in an amount effective to increase the melting temperature (Tₘ) of the phospholipid such that the pharmaceutical composition exhibits a Tₘ which is greater than its storage temperature (Tₘ) by at least about 200 C., such as at least about 40° C. The molar ratio of polyvalent cation to phospholipid may be at least about 0.05:1, such as about 0.05:1 to about 2.0:1 or about 0.25:1 to about 1.0:1. An example of the molar ratio of polyvalent cation:phospholipid is about 0.50:1. When the polyvalent cation is calcium, it may be in the form of calcium chloride. Although metal ion, such as calcium, is often included with phospholipid, none is required.

As noted above, the pharmaceutical composition may include one or more surfactants. For instance, one or more surfactants may be in the liquid phase with one or more being associated with solid particles or particles of the composition. By “associated with” it is meant that the pharmaceutical compositions may incorporate, adsorb, absorb, be coated with, or be formed by the surfactant. Surfactants include, but are not limited to, fluorinated and nonfluorinated compounds, such as saturated and unsaturated lipids, nonionic detergents, nonionic block copolymers, ionic surfactants, and combinations thereof. It should be emphasized that, in addition to the aforementioned surfactants, suitable fluorinated surfactants are compatible with the teachings herein and may be used to provide the desired preparations. Examples of ionic surfactants include, but are not limited to, sodium sulfosuccinate, and fatty acid soaps.

Examples of amino acids include, but are not limited to hydrophobic amino acids. Use of amino acids as pharmaceutically acceptable excipients is known in the art as disclosed in WO 95/31479, WO 96/32096, and WO 96/32149, which are incorporated herein by reference in their entireties.

Examples of carbohydrates include, but are not limited to, monosaccharides, disaccharides, and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins, and maltodextrins.

Examples of buffers include, but are not limited to, tris or citrate.

Examples of salts include, but are not limited to, sodium chloride, salts of carboxylic acids, (e.g., sodium citrate, sodium ascorbate, magnesium gluconate, sodium gluconate, tromethamine hydrochloride, etc.), ammonium carbonate, ammonium acetate, ammonium chloride, and the like.

Examples of organic solids include, but are not limited to, camphor, and the like.

The pharmaceutical composition of one or more embodiments of the present disclosure may also include a biocompatible, such as biodegradable polymer, copolymer, or blend or other combination thereof. In this respect useful polymers comprise polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.). Those skilled in the art will appreciate that, by selecting the appropriate polymers, the delivery efficiency of the composition and/or the stability of the dispersions may be tailored to optimize the effectiveness of the antiarrhythmic pharmaceutical agent(s). Examples of block copolymers include, but are not limited to, diblock and triblock copolymers of polyoxyethylene and polyoxypropylene, including poloxamer 188 (Pluronic™ F-68), poloxamer 407 (Pluronic™ F-127), and poloxamer 338.

For solutions, the compositions may include one or more osmolality adjuster, such as sodium chloride. For instance, sodium chloride may be added to solutions to adjust the osmolality of the solution. In one or more embodiments, an aqueous composition consists essentially of the antiarrhythmic pharmaceutical agent, the osmolality adjuster, and water.

Solutions may also comprise a buffer or a pH adjusting agent, typically a salt prepared from an organic acid or base. Representative buffers comprise organic acid salts of citric acid, lactic acid, ascorbic acid, gluconic acid, carbonic acid, tartaric acid, succinic acid, acetic acid, or phthalic acid, Tris, tromethamine hydrochloride, or phosphate buffers. Thus, the buffers include citrates, phosphates, phthalates, and lactates. The compositions typically have a pH ranging from 3.5 to 8.0, such as from 4.0 to 7.5, or 4.5 to 7.0, or 5.0 to 6.5.

Besides the above mentioned pharmaceutically acceptable excipients, it may be desirable to add other pharmaceutically acceptable excipients to the pharmaceutical composition to improve particle rigidity, production yield, emitted dose and deposition, shelf-life, and patient acceptance. Such optional pharmaceutically acceptable excipients include, but are not limited to: coloring agents, taste masking agents, buffers, hygroscopic agents, antioxidants, and chemical stabilizers. Further, various pharmaceutically acceptable excipients may be used to provide structure and form to the particle compositions (e.g., latex particles). In this regard, it will be appreciated that the rigidifying components can be removed using a post-production technique such as selective solvent extraction.

The compositions of one or more embodiments of the present disclosure may take various forms, such as solutions, dry powders, reconstituted powders, suspensions, or dispersions comprising a non-aqueous phase, such as propellants (e.g., chlorofluorocarbon, hydrofluoroalkane).

For dry powders, the moisture content is typically less than about 15 wt %, such as less than about 10 wt % less than about 5 wt %, less than about 2 wt %, less than about 1 wt %, or less than about 0.5 wt %. Such powders are described in WO 95/24183, WO 96/32149, WO 99/16419, WO 99/16420, and WO 99/16422, which are incorporated herein by reference in their entireties. The moisture content is, at least in part, dictated by the composition and is controlled by the process conditions employed, e.g., inlet temperature, feed concentration, pump rate, and blowing agent type, concentration and post drying. Reduction in bound water leads to significant improvements in the dispersibility and flowability of phospholipid based powders, leading to the potential for highly efficient delivery of powdered lung surfactants or particle composition comprising active agent dispersed in the phospholipid. The improved dispersibility allows simple passive DPI devices to be used to effectively deliver these powders.

In some versions, the pharmaceutical composition comprises particles having a mass median diameter less than about 20 μm, such as less than about 10 μm, less than about 7 μm, or less than about 5 μm. The particles may have a mass median aerodynamic diameter ranging from about 1 μm to about 6 μm, such as about 1.5 μm to about 5 μm, or about 2 μm to about 4 μm. If the particles are too large, a larger percentage of the particles may not reach the lungs. If the particles are too small, a larger percentage of the particles may be exhaled.

### Route of Administration

The pharmaceutical compositions described herein may be administered using a dry powder inhaler. A specific version of a dry powder aerosolization apparatus is described in U.S. Pat. Nos. 4,069,819 and 4,995,385, which are incorporated herein by reference in their entireties. Another useful device, which has a chamber that is sized and shaped to receive a capsule so that the capsule is orthogonal to the inhalation direction, is described in U.S. Pat. No. 3,991,761, which is incorporated herein by reference in its entirety. As also described in U.S. Pat. No. 3,991,761, a puncturing mechanism may puncture both ends of the capsule. In another version, a chamber may receive a capsule in a manner where air flows through the capsule as described for example in U.S. Pat. Nos. 4,338,931 and 5,619,985, which are incorporated herein by reference in their entireties. In another version, the aerosolization of the pharmaceutical composition may be accomplished by pressurized gas flowing through the inlets, as described for example in U.S. Pat. Nos. 5,458,135; 5,785,049; and 6,257,233, or propellant, as described in WO 00/72904 and U.S. Pat. No. 4,114,615, which are incorporated herein by reference. These types of dry powder inhalers are generally referred to as active dry powder inhalers.

Other dry powder inhalers include those available from Boehringer Ingelheim (e.g., Respimat inhaler), Hovione (e.g., FlowCaps inhaler), Plastiape (e.g., Osmohaler inhaler), and MicroDose. The present disclosure may also utilize condensation aerosol devices, available from Alexza, Mountain View, Calif. Yet another useful inhaler is disclosed in WO 2008/051621, which is incorporated herein by reference in its entirety.

The pharmaceutical compositions described herein may also be administered using an aerosolization device. The aerosolization device may be a nebulizer, a metered dose inhaler, a liquid dose instillation device, or a dry powder inhaler. The aerosolization device may comprise the extrusion of the pharmaceutical preparation through micron or submicron-sized holes with subsequent Rayleigh break-up into fine droplets. The pharmaceutical composition may be delivered by a nebulizer as described in WO 99/16420, by a metered dose inhaler as described in WO 99/16422, by a liquid dose instillation apparatus as described in WO 99/16421, and by a dry powder inhaler as described in U.S. Published Application Nos. 20020017295 and 20040105820, WO 99/16419, WO 02/83220, and U.S. Pat. No. 6,546,929, which are incorporated herein by reference in their entireties. As such, an inhaler may comprise a canister containing the particles or particles and propellant, and wherein the inhaler comprises a metering valve in communication with an interior of the canister. The propellant may be a hydrofluoroalkane.

The formulations of the present disclosure may be administered with nebulizers, such as that disclosed in PCT WO 99/16420, the disclosure of which is hereby incorporated in its entirety by reference, in order to provide an aerosolized medicament that may be administered to the pulmonary air passages of a patient in need thereof. Nebulizers are known in the art and could easily be employed for administration of the claimed formulations without undue experimentation. Breath activated or breath-actuated nebulizers, as well as those comprising other types of improvements which have been, or will be, developed are also compatible with the formulations of the present disclosure and are contemplated as being within the scope thereof.

In some cases, the nebulizer is a breath activated or breath-actuated nebulizer. In some cases, the nebulizer is a hand-held inhaler device (e.g., AeroEclipse® II Breath Actuated Nebulizer (BAN)). In some cases, the nebulizer has a compressed air source. In some cases, the nebulizer converts liquid medication into an aerosol. In some cases, the nebulizer converts liquid medication into an aerosol by extruding the pharmaceutical preparation through micron or submicron-sized holes. In some cases, the nebulizer converts liquid medication into an aerosol so it can be inhaled into the lungs. In some cases, the nebulizer is a small volume nebulizer. In some cases, the nebulizer is a small volume jet nebulizer. In some cases, aerosolized medication is only produced when inhaled through the device. In some cases, the medication is contained in the cup between breaths or during breaks in treatment. In some cases, the medication is contained in the cup until ready to be inhaled.

Nebulizers impart energy into a liquid pharmaceutical formulation to aerosolize the liquid, and to allow delivery to the pulmonary system, e.g., the lungs, of a patient. A nebulizer comprises a liquid delivery system, such as a container having a reservoir that contains a liquid pharmaceutical formulation. The liquid pharmaceutical formulation generally comprises an active agent that is either in solution or suspended within a liquid medium.

In one type of nebulizer, generally referred to as a jet nebulizer, compressed gas is forced through an orifice in the container. The compressed gas forces liquid to be withdrawn through a nozzle, and the withdrawn liquid mixes with the flowing gas to form aerosol droplets. A cloud of droplets is then administered to the patient's respiratory tract.

In another type of nebulizer, generally referred to as a vibrating mesh nebulizer, energy, such as mechanical energy, vibrates a mesh. This vibration of the mesh aerosolizes the liquid pharmaceutical formulation to create an aerosol cloud that is administered to the patient's lungs. In another type of nebulizer, the nebulizing comprises extrusion through micron or submicron-sized holes followed by Rayleigh break-up into fine droplets.

The pharmaceutical composition of one or more embodiments of the present disclosure typically has improved emitted dose efficiency. Accordingly, high doses of the pharmaceutical composition may be delivered using a variety of aerosolization devices and techniques. The emitted dose (ED) of the particles of the present disclosure may be greater than about 30%, such as greater than about 40%, greater than about 50%, greater than about 60%, or greater than about 70%.

### Methods

Provided herein are methods that comprise any of the compositions disclosed herein. In some cases, a method of treating a substance use disorder is provided. In some cases, a method of treating anxiety is provided. In some cases, a method of treating depression is provided. In some cases, a method is effective in delaying or preventing relapse of substance use. In some cases, a method is effective in reducing a symptom of one or more of anxiety, depression, or addiction. In some cases, a method comprises administering a therapeutically effective amount of 5-methoxy-N,N-dimethyltryptamine (5-MeO-DMT), or a pharmaceutically acceptable derivative or salt thereof to a subject in need.

In some cases, a substance use disorder comprises use of a stimulant. In some cases, a substance use disorder comprises use of a sedative. In some cases, the stimulant is selected from the group consisting of cocaine, nicotine, methamphetamine, amphetamine, ecstasy, and any combination thereof. In some cases, the stimulant is cocaine. In some cases, the sedative is selected from the group consisting of barbiturates, benzodiazepines, antihistamines, antidepressants, opioids, antipsychotics, alcohol, and any combination thereof. In some cases, the sedative is heroin.

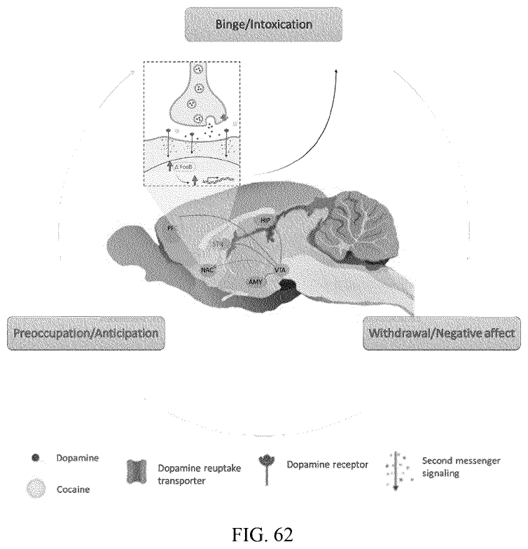

In some cases, a subject that is administered a composition of the disclosure is experiencing one or more of a binge, intoxication, withdrawal, preoccupation, anticipation, and any combination thereof. In some cases, to determine the presence of the aforementioned states, neuroplastic changes are examined according to FIG. 62. Neuroplastic changes can be evaluated by way of a change in a biomarker such as dopamine, ΔFosB, or both as compared to an otherwise comparable subject lacking the diseased mental state. In some cases, described is a method of modulating gene or protein expression of a biomarker in a subject suffering from sustained substance exposure. In some cases, a biomarker is selected from the group consisting of. FosB, ΔFosB, cAMP response element binding protein (CREB), histone methyltransferase (G9a), histone H3 lysine 9 (H3K9), metabotropic glutamate receptor (mGluR), glucocorticoid receptor (GR), 5-HT_{1A} Receptor (5-HT_{1A}R), and brain-derived neurotrophic factor (BDNF). In some cases, a biomarker is FosB and its gene or protein expression is elevated. In some cases, a biomarker is FosB and its gene or protein expression is elevated in nucleus accumbens about 20-30, 15-25, 20-35, or 22-26 hours after the sustained substance exposure. In some cases, a biomarker is FosB and its gene or protein expression is elevated in nucleus accumbens about 24 hours after the sustained substance exposure. In some cases, a biomarker is G9a, and its gene or protein expression is elevated in dorsal striatum about 20-30, 15-25, 20-35, or 22-26 hours after the sustained substance exposure. In some cases, a biomarker is G9a, and its gene or protein expression is elevated in dorsal striatum about 24 hours after the sustained substance exposure.

In some cases, a method is effective in reducing an elevated gene or protein expression of FosB in nucleus accumbens as compared to an otherwise comparable method lacking administration of 5-MeO-DMT. In some cases, the gene or protein expression is reduced by at least about 1-fold, 2-fold, 3-fold, 4-fold, 5-fold, 10-fold, 20-fold, 40-fold, 60-fold, 80-fold, 100-fold, 120-fold, 140-fold, 160-fold, 180-fold, 200-fold, 220-fold, 240-fold, 260-fold, 280-fold, or up to about 300-fold post administration as compared to a subject not administered 5-MeO-DMT. In some cases, the gene or protein expression is reduced by at least about 10%, 15%, 20%, 25%, 50%, 60%, 70%, 80%, 90%, 95%, compared to a subject not administered the 5-MeO-DMT.

In some cases, a method is effective in restoring neuroplastic architecture of a brain tissue as compared to an otherwise comparable method lacking administration of 5-MeO-DMT.

In some cases, administration of a composition comprising 5-MeO-DMT is effective in reducing a symptom of one or more of anxiety, depression, addiction. In some cases, a symptom is reduced by at least about 1-fold, 2-fold, 3-fold, 4-fold, 5-fold, 10-fold, 20-fold, 40-fold, 60-fold, 80-fold, 100-fold, 120-fold, 140-fold, 160-fold, 180-fold, 200-fold, 220-fold, 240-fold, 260-fold, 280-fold, or up to about 300-fold post administration as compared to a subject not administered 5-MeO-DMT. In some cases, a symptom is reduced by at least about 10%, 15%, 20%, 25%, 50%, 60%, 70%, 80%, 90%, 95%, compared to a subject not administered the 5-MeO-DMT.

In some cases, administration of 5-MeO-DMT is effective in achieving a clinical endpoint for treating a substance use disorder.

In some cases, a method provided can further comprise administering to a subject a therapeutically effective amount of an antidote reversal agent. An antidote reversal agent can be selected from the group consisting of ketanserin, rapamycin, pizotifen, spiperone, ritanserin, WAY100635, and ANA-12. In some cases, a method comprises calculating a dose of an antidote reversal agent to be delivered.

In some cases, a method comprises obtaining a sample from a subject. A sample can be obtained at any time. In some cases, a sample comprises urine, blood, saliva, hair, or combinations thereof. In some cases, a sample is a urine sample. In some cases, a sample is a saliva sample.

In some cases, a composition of the disclosure is administered in a therapeutically effective amount. In some cases, a therapeuticall effective amount is from about 1 mg/kg to about 50 mg/kg. In some cases, the therapeutically effective amount is from about 5 mg/kg to about 25 mg/kg. In some cases, the therapeutically effective amount is about 10 mg/kg or 20 mg/kg. In some cases, the therapeutically effective amount is at least about or at most about: 1 mg/kg, 3 mg/kg, 5 mg/kg, 7 mg/kg, 9 mg/kg, 11 mg/kg, 13 mg/kg, 15 mg/kg, 17 mg/kg, 19 mg/kg, 21 mg/kg, 23 mg/kg, 25 mg/kg, 27 mg/kg, 29 mg/kg, 31 mg/kg, 33 mg/kg, 35 mg/kg, 37 mg/kg, 39 mg/kg, 41 mg/kg, 43 mg/kg, 45 mg/kg, 47 mg/kg, 49 mg/kg, 51 mg/kg, 53 mg/kg, 55 mg/kg, 57 mg/kg, 59 mg/kg, 61 mg/kg, 63 mg/kg, 65 mg/kg, 67 mg/kg, 69 mg/kg, 71 mg/kg, 73 mg/kg, or 75 mg/kg.

In some cases, any of the described compositions comprising 5-MeO-DMT or pharmaceutically acceptable derivative or salt thereof, can be administered via a delivery route selected from the group consisting of oral, intravenous, intraperitoneal, intramuscular, intradermal, subcutaneous, intra-arteriole, intraventricular, intracranial, intralesional, intrathecal, topical, transmucosal, intranasal, and a combination thereof.

### NUMBERED EMBODIMENTS

Notwithstanding the appended claims, the following numbered embodiments also form part of the instant disclosure.

Embodiment 1: A method of treating a substance use disorder in a subject suffering from sustained substance exposure, comprising: administering to the subject a therapeutically effective amount of 5-methoxy-N,N-dimethyltryptamine (5-MeO-DMT), or a pharmaceutically acceptable derivative or salt thereof.

Embodiment 2: The method of embodiment 1, wherein the administering 5-MeO-DMT results in a clinical endpoint for treating the substance use disorder.

Embodiment 3: The method of embodiment 1 or 2, wherein the clinical endpoint comprises reducing self-administration of the substance, decreasing a propensity for relapse, reducing an effect of substance withdrawal, or any combination thereof.

Embodiment 4: A method of reducing anxiety or depression of a subject suffering from sustained substance exposure, comprising: administering to the subject a therapeutically effective amount of 5-methoxy-N,N-dimethyltryptamine (5-MeO-DMT), or a pharmaceutically acceptable derivative or salt thereof.

Embodiment 5: The method of any one of embodiments 1-4, wherein the method modulates gene expression of a biomarker in the subject.

Embodiment 6: A method of modulating gene or protein expression of a biomarker in a subject suffering from sustained substance exposure, comprising: administering to the subject a therapeutically effective amount of 5-methoxy-N,N-dimethyltryptamine (5-MeO-DMT), or a pharmaceutically acceptable derivative or salt thereof.

Embodiment 7: The method of any one of embodiments 1-6, wherein the substance comprises a stimulant or a sedative.

Embodiment 8: The method of embodiment 7, wherein the stimulant is selected from the group consisting of cocaine, nicotine, methamphetamine, amphetamine, ecstasy, and any combination thereof.

Embodiment 9: The method of embodiment 8, wherein the stimulant is cocaine.

Embodiment 10: The method of embodiment 7, wherein the sedative is selected from the group consisting of barbiturates, benzodiazepines, antihistamines, antidepressants, opioids, antipsychotics, alcohol, and any combination thereof.

Embodiment 11: The method of embodiment 10, wherein the sedative is heroin.

Embodiment 12: The method of any one of embodiments 5-11, wherein the biomarker is measured in a blood or urine sample from the subject.

Embodiment 13: The method of any one of embodiments 5-12, wherein the biomarker is selected from the group consisting of FosB, ΔFosB, cAMP response element binding protein (CREB), histone methyltransferase (G9a), histone H3 lysine 9 (H3K9), metabotropic glutamate receptor (mGluR), glucocorticoid receptor (GR), 5-HT_{1A} Receptor (5-HT_{1A}R), and brain-derived neurotrophic factor (BDNF).

Embodiment 14: The method of embodiment 13, wherein the biomarker is FosB, and its gene or protein expression is elevated in nucleus accumbens 24 hours after the sustained substance exposure.

Embodiment 15: The method of embodiment 14, wherein the method reduces the elevated gene or protein expression of FosB in nucleus accumbens.

Embodiment 16: The method of embodiment 13, wherein the biomarker is G9a, and its gene or protein expression is elevated in dorsal striatum 24 hours after the sustained substance exposure.

Embodiment 17: The method of embodiment 16, wherein the method reduces the elevated gene or protein expression of G9a in dorsal striatum.

Embodiment 18: The method of embodiment 13, wherein the biomarker is ΔFosB, and its gene or protein expression is elevated in dorsal striatum 24 hours after the sustained substance exposure.

Embodiment 19: The method of embodiment 18, wherein the method reduces the elevated gene or protein expression of ΔFosB in dorsal striatum.

Embodiment 20: The method of embodiment 13, wherein the biomarker is 5-HT_{1A}R, and its gene or protein expression is reduced in hippocampus 24 hours after the sustained substance exposure.

Embodiment 21: The method of embodiment 20, wherein the method increases the reduced gene or protein expression of 5-HT_{1A}R in hippocampus.

Embodiment 22: The method of embodiment 13, wherein the biomarker is G9a, and its gene or protein expression is reduced in hippocampus 24 hours after the sustained substance exposure.

Embodiment 23: The method of embodiment 22, wherein the method increases the reduced gene or protein expression of G9a in hippocampus.

Embodiment 24: The method of embodiment 13, wherein the biomarker is ΔFosB, and its gene or protein expression is elevated in amygdala 24 hours after the sustained substance exposure.

Embodiment 25: The method of embodiment 24, wherein the method reduces the elevated gene or protein expression of ΔFosB in amygdala.

Embodiment 26: The method of embodiment 13, wherein the biomarker is 5-HT_{1A}R, and its gene or protein expression is reduced in amygdala 24 hours after the sustained substance exposure.

Embodiment 27: The method of embodiment 26, wherein the method increases the reduced gene or protein expression of 5-HT_{1A}R in amygdala.

Embodiment 28: The method of embodiment 13, wherein the biomarker is G9a, and its gene or protein expression is reduced in amygdala 24 hours after the sustained substance exposure.

Embodiment 29: The method of embodiment 28, wherein the method increases the reduced gene or protein expression of G9a in amygdala.

Embodiment 30: The method of any one of embodiments 1-29, wherein the therapeutically effective amount is from about 1 mg/kg to about 50 mg/kg.

Embodiment 31: The method of embodiment 30, wherein the therapeutically effective amount is from about 5 mg/kg to about 25 mg/kg.

Embodiment 32: The method of embodiment 31, wherein the therapeutically effective amount is about 10 mg/kg or 20 mg/kg.

Embodiment 33: The method of any one of embodiments 1-32, further comprising administering to the subject a therapeutically effective amount of an antidote reversal agent.

Embodiment 34: The method of embodiment 33, wherein the antidote reversal agent is selected from the group consisting of ketanserin, rapamycin, pizotifen, spiperone, ritanserin, WAY100635, and ANA-12.

Embodiment 35: The method of embodiment 33, further comprising calculating a dose of the antidote reversal agent to be delivered.

Embodiment 36: The method of any one of embodiments 1-35, wherein the 5-MeO-DMT or pharmaceutically acceptable derivative or salt thereof is administered to the subject via a delivery route selected from the group consisting of oral, intravenous, intraperitoneal, intramuscular, intradermal, subcutaneous, intra-arteriole, intraventricular, intracranial, intralesional, intrathecal, topical, transmucosal, intranasal, and a combination thereof.

Embodiment 37: A kit, comprising: (a) 5-Methoxy-N,N-dimethyltryptamine (5-MeO-DMT), or pharmaceutically acceptable derivative or salt thereof; and (b) an antidote reversal agent.

Embodiment 38: The kit of embodiment 37, wherein the antidote reversal agent is selected from the group consisting of ketanserin, rapamycin, pizotifen, spiperone, ritanserin, WAY100635, and ANA-12.

Embodiment 39: The kit of embodiment 37, further comprising a digital device configured to calculate a dose of the antidote reversal agent to be delivered.

### EXAMPLES

### Example 1—chronic cocaine f 5-MeO-DMT molecular study

Male Wistar rats aged 7 weeks were acclimatized to the facility for 1 week prior to the beginning of the study. Animals were administered cocaine hydrochloride (15 mg/kg, Sigma) or saline by i.p. injection once daily for 7 days. For the subsequent 7 days animals receiving cocaine were administered a higher dose 20 mg/kg i.p. to encapsulate the increased drug use seen following prolonged drug use in human addicts (Piazza, P. V. & V. Deroche-Gamonet (2013) A multistep general theory of transition to addiction. Psychopharmacology, 229, 387-413). One-hour post last cocaine animals received a vehicle, low (10 mg/kg) or high (20 mg/kg) dose of 5-MeO-DMT (CarboSynth). Animals were euthanized 23- or 119-hours following vehicle or 5-MeO-DMT administration.

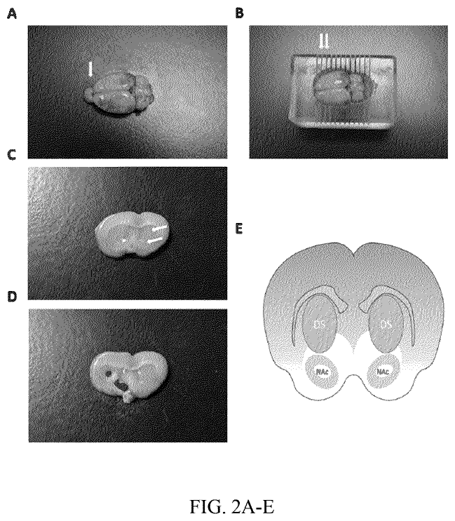

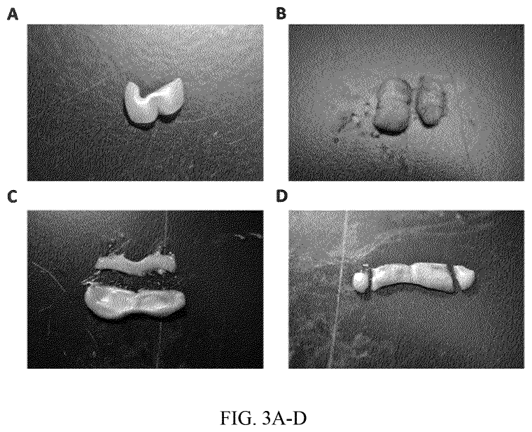

Following euthanasia brain regions of interest were isolated. Briefly, the olfactory bulbs were removed before the brain was placed in a rat brain matrix (FIG. 2A). Sterile blades were inserted at positions 2 and 3 (FIG. 2B) to extract a coronal slice containing the Bregma+˜0.2-+˜2.2 coordinates (FIG. 2C). From this tissue slice a blunted 12 G needle was used to punch dissect the NAc and dorsal striatum (DS) (FIG. 2D-2E) identified using the rat brain atlas (Panxinos and Watson 2nd edition). The PFC, hippocampus (Hip) and amygdala (Amyg) were then blunt dissected (FIG. 3A-3D). All regions were frozen in liquid nitrogen and stored at −80° C. until processed.

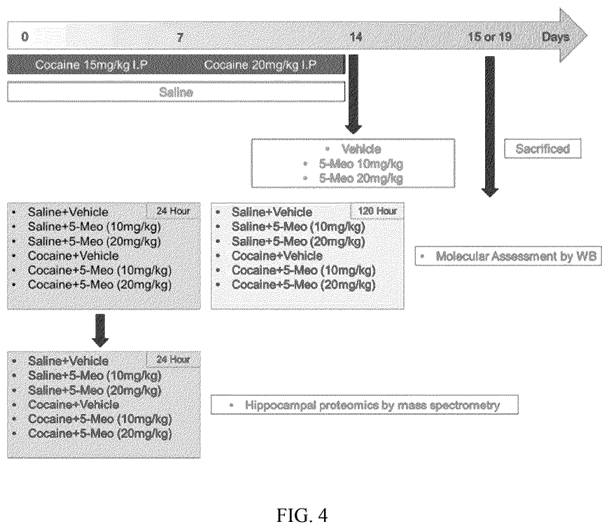

The study design is summarized in FIG. 4. There were 12 treatment groups arising from this study each comprised of 6 animals, they were as follows; saline+vehicle, saline+10 mg/kg 5-MeO-DMT, saline+20 mg/kg 5-MeO-DMT, cocaine+vehicle, cocaine+10 mg/kg 5-MeO-DMT and cocaine+20 mg/kg 5-MeO-DMT for 24- and 120-hours post cocaine/saline. These treatment groups were used for the molecular assessment of addiction-linked markers. Hippocampal protein from the treatment groups saline+vehicle, saline+10 mg/kg 5-MeO-DMT, saline+20 mg/kg 5-MeO-DMT, cocaine+vehicle, cocaine+10 mg/kg 5-MeO-DMT and cocaine+20 mg/kg 5-MeO-DMT collected 24 hours after the last cocaine/saline exposure were further analyzed by mass spectrometry.

### Example 2—Analysis Methods

Determination of protein concentration—a bicinchoninic acid assay (BCA, ThermoFisher) was conducted to determine the total protein concentration in tissue lysates following manufacturer's instructions. Briefly, bovine serum albumin (BSA) standards were prepared in a serial dilution (0-2.0 mg protein/ml) and 200 μl working reagent (50:1, reagent A: reagent B) was added to each of the standards and samples in a 96 well plate (Greiner). For tissue lysates a 1:10 dilution was performed with cell lysis buffer to ensure absorbance values lay within the working range established by the standards. All samples and standards were added in duplicate. The plate was covered and incubated for 30 mins at 37° C. before absorbance was read at 562 nm on a plate reader (SpectraMax M3). The absorbance values were used to form a standard curve of known protein concentrations and protein concentration of samples were derived using the equation of the line. All samples were standardized to the lowest protein concentration using distilled H20 (dH₂O).

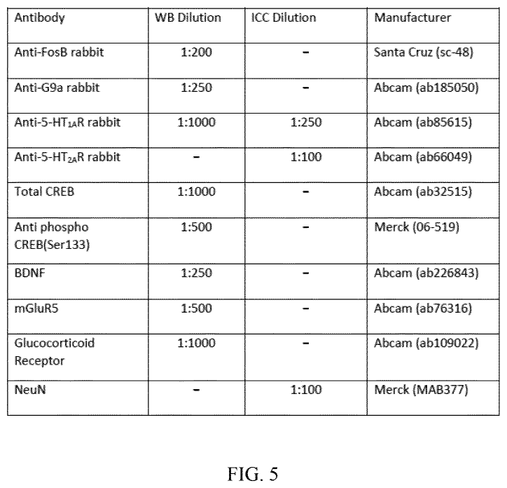

SDS-PAGE and Western Blotting—Following equalisation of protein concentrations, 6× sample loading buffer (15% SDS, 15% μ-mercaptoethanol, 50% glycerol, 0.01% bromophenol blue, 0.125 mM Tris) was added to each sample using a dilution factor of 1:5 of the total volumes before boiling at 100° C. for 5 mins. Samples were stored at −20° C. until use. One dimensional sodium-dodecyl-sulphate polyacrylamide gel electrophoresis (SDS-PAGE) was used for protein separation based on molecular weight. The percentage of acrylamide in the resolving or stacking gel was determined by the molecular weight of the protein of interest. In this instance all gels consisted of a 5% stacking gel and a 10% resolving gel with the exception of mGluR5, G9a (8%) and H3K9Me2 (12%). 20 μg of protein was loaded and resolved using SDS-PAGE. Gel electrophoresis was conducted at 80V while the protein was in the stacking gel, once it migrated to the resolving gel the voltage was increased to 120V and ran for 90 mins. Samples were transferred to a 0.45 μm polyvinylidene difluoride (PVDF) membrane (Analab) for 1 hour at 100V and blocked in 5% milk blocking solution made in tris-buffered saline containing tween®-20 (TBS-T, Sigma) for 90 mins. Primary antibodies (FIG. 5) were diluted in 5% BSA in TBS-T and incubated with membranes overnight at 4° C. or for 2 hours at room temperature. After incubation with primary antibodies the membranes were washed in TBS-T for 4×10 minutes before incubation with 1:2000 anti-rabbit and 1:2000 anti-mouse Alexa Fluro® conjugated secondary antibodies (Invitrogen) in 2% milk for 1 hour at room temperature. Membranes were washed in TBS-T for 4×10 minutes and in TBS for 1×10-minute wash. Proteins were visualized using Odyssey® infrared imaging system. Protein expression was quantified using Image Studio Lite (Version 5.2) and expression intensities were normalized to a house keeping protein, α-Tubulin or β-actin.

Statistical analysis—GraphPad Prism 5.0 was used for statistical analysis and graphical representation of all data. All data are presented as mean±SEM. For normally distributed data a two-way ANOVA with Bonferroni posthoc test was used to determine if there were statistically significant differences in at any between treatment groups. For additional analysis a one-way ANOVA with Dunnett's post-hoc test was used to determine statistical significance. If the data failed the Kolmogorov Smirnov test for normality it was analyzed by the non-parametric Kruskal-Wallis test followed by Dunn's post hoc test. A threshold for statistical significance was set at P<0.05 for all statistical tests performed.

### Example 3—Liquid Chromatography Tandem Mass Spectrometry (LC-MS/MS)

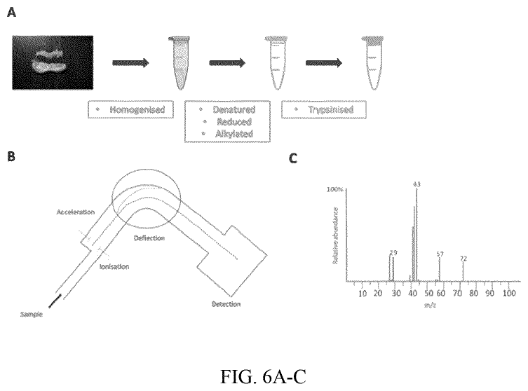

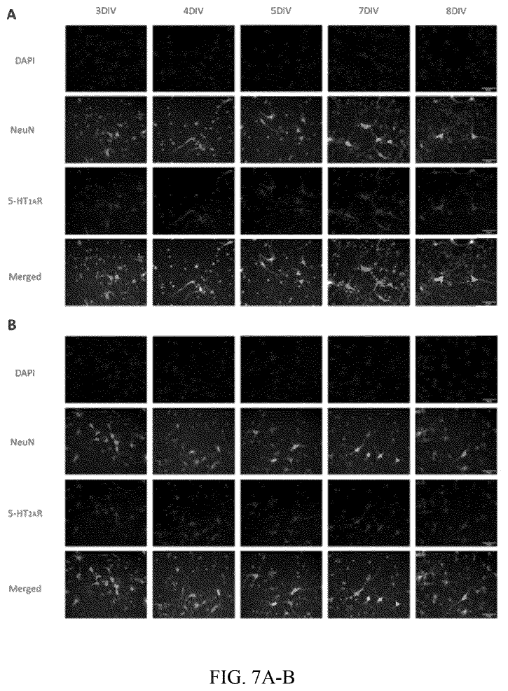

The LC-MS/MS study is summarized in FIG. 6A-6C. Protein was extracted from hippocampal tissue dissected 24 hours post last cocaine/saline in the cocaine±5-MeO-DMT study as described. Due to incompatibilities between components of the lysis buffer in the High-Performance Liquid Chromatography (HPLC) mass spectrometer the samples were first precipitated in 100% (w/v) Trichloroacetic acid (TCA). 1 volume of TCA stock was added to 4 volumes of protein sample. The mixture was incubated for 10 mins at 4° C. before centrifugation at 14,000 RPM for 5 mins. The supernatant was discarded and the pellet was washed in acetone. The acetone was then discarded and the pellet was air-dried before resuspension in 6M urea (Sigma) made in 50 mM ammonium bicarbonate (ABC, Sigma). 100 mM dithiothreitol (DTT) was added to the sample for a final concentration of 5 mM and the sample was incubated while vortexing at 60° C. for 30 mins. Next, 200 mM iodoacetemide (IAA, Sigma) solution was added and the sample was incubated in the dark at room temperature for 30 mins. The sample was then diluted with 50 mM ABC to give a final concentration of ≤2M Urea before the addition of trypsin. 50 μl of reduced, alkylated sample was added to a vial of trypsin singles, proteomic grade (Sigma) and incubated at 37° C. overnight while vortexing. 1% acetic acid (AA) was added to the sample to stop the trypsination the following day. Sample clean-up was performed using Stage Tips for a 10 μl pipette with a 0.6 μl C18 resin (Merck) and clean up was carried out as follows; Tip was equilibrated (10 μl of 50% acetonitrile (ACN) in 0.1% trifluoroacetic acid (TFA) was aspirated into the tip and dispensed to waste x2, 10 μl of 0.1% TFA was aspirated and dispensed to waste x2), peptides were bound to resin and washed (10 μl of sample was aspirated and dispensed x5 cycles, 10 μl of 0.1% TFA was aspirated and dispensed to waste x2) before being eluted into a collecting tube containing 30 μl of 50% ACN in 0.1% TFA (aspirate and dispense the sample through Stage Tip x3). This process was repeated three times for each sample using the same Stage Tip and collection tube. Samples were dried in a vacuum centrifuge for 30 mins at 60° C. and resuspended in 30 μl of Buffer A (97% water, 2.5% ACN and 0.5% AA) and stored at −20° C.

Digested samples were run on a Thermo Scientific Q Exactive mass spectrometer connected to a Dionex Ultimate 3000 (RSLCnano) chromatography system. Tryptic peptides were resuspended in 0.1% formic acid. Each sample was loaded onto a fused silica emitter (75 μm ID, pulled using a laser puller (Sutter Instruments P2000)), packed with Recrocil Pur C18 (1.9 μm) reverse phase media and was separated by an increasing acetonitrile gradient over 47 mins at a flow rate of 250 nL/min. The mass spectrometer was operated in positive ion mode with a capillary temperature of 320° C., and with a potential of 2300V applied to the frit. All readings were acquired with the mass spectrometer operating in automatic data dependent switching mode. A high resolution (70,000) MS scan was performed using the Q Exactive to select the most intense ions prior to MS/MS analysis using Higher-energy collisional dissociation (HCD). To identify peptides and proteins the MS/MS spectra were searched against the rat UniProt database. The database searches were performed with carbamidomethylation (C) as a fixed modification and acetylation (protein N terminus) and oxidation (M) as variable modifications. Filtering by a false discovery rate (FDR) was applied for peptides and proteins (0.01). For the generation of label-free quantitative (LFQ) ion intensities for protein profiles, signals of corresponding peptides in different LCMS/MS runs were matched by MaxQuant.

The Perseus statistical software contained in the MaxQuant package was used to analyze ion intensities (LFQ Intensity). Protein identities were filtered to eliminate the identifications from the reverse database, only identified by site and common contaminants. The data was log 2 transformed and filtered based on valid values such that proteins that were not present in a minimum of 4 samples in at least one treatment group were eliminated. Principle component analysis (PCA) was performed on the data. Two sample t-tests were applied to determine if mean LFQ intensity values of treatment groups were significantly different from one another. Statistical cut offs used for each comparison are p<0.05 with a fold change in expression of ±20%. Hierarchical clustering of differentially expressed proteins was carried out in Cluster 3.0 using Euclidean distance measures and average linkage. Pathway analysis was carried out using PANTHER (Protein Analysis Through Evolutionary Relationships) classification system (version 14.1). PANTHER determines statistical overrepresentation in lists of differentially expressed proteins or genes. The PANTHER system was developed to classify gene and protein function but has evolved to serve as an online resource for the analysis of gene function on a genome wide scale. It is composed of three modules, a protein library containing all protein encoding genes from a number of organisms, the PANTHER module containing 176 expert curated pathways and finally the website tool suite containing a collection of bioinformatics tools and software. For this analysis the gene list analysis tool was used to perform a statistical overrepresentation test. This test compares the input list to a reference list (Rattus norvegicus) in order to statistically determine over- or under-representation using Fisher's exact test. The input list was a tab delineated file containing differentially expressed protein names (and subsequently gene IDs). Bonferroni correction for multiple comparisons was applied where indicated.

### Example 4—In Vitro Assessment of Cocaine and 5-MeO-DMT on Cultured Hippocampal Neuron Structure

Primary Culture—Primary hippocampal neurons were derived from the hippocampi of embryonic day 18 (E18) rat embryos, as described previously (Seibenhener, M. L. & M. W. Wooten (2012) Isolation and Culture of Hippocampal Neurons from Prenatal Mice. Journal of Visualized Experiments: JoVE, 3634). Briefly, the brains were removed from the skull and stored in cooled Hank's Balanced Salt Solution (HBSS). The hippocampus was isolated under a dissection microscope and digested using 0.05% Trypsin (Gibco) in HBSS for 10 mins after which trypsinisation was stopped by the addition of 2% foetal calf serum (Gibco). Isolated cells were suspended in plating media and viable cells were counted with a haemocytometer using trypan blue (Gibco) staining. Cells were plated at a density of 1×10⁶ cells/well on 12-well plates coated with 0.02% Poly-DL-ornithine hydrobromide (Sigma). After plating, cells were incubated at 37° C. in humidified air with 5% C02. Half medium changes were performed every 2-3 days with L-glutamic acid no longer included in the media after 3 DIV.

Immunocytochemistry—Primary hippocampal neurons grown on 18 mm diameter glass coverslips were fixed in 70% ethanol (EtOH) for 20 mins. After fixing, cells were permeabilised in 0.2% Triton X-100 (Thermo Fisher) in PBS and washed in PBS. Cells were blocked in 1% BSA (Sigma) in PBS for 45 mins. Following blocking, cells were incubated with primary antibodies in 5% BSA and 5% normal goat serum (Agilent) for 2 hours in a humidity chamber at room temperature or overnight at 4° C. Cells were rinsed in PBS and incubated with 1 ug/ml 4′,6-Diamidine-2′-phenylindole dihydrochloride (DAPI) (Thermo Fisher) for 10 mins. Coverslips were mounted on microscope slides using mounting medium containing mowiol 4-88 (Sigma), stored in the dark at 4° C. and were imaged within 7 days of mounting. Images were captured on a Zeiss AxioImager M1 fluorescent microscope using a x40 oil immersion lens. 10 images were taken per coverslip and images were analyzed by a customized R script (Appendix I) using EBImage by Bioconductor Ver 3.7. Briefly, DAPI staining was used as a nuclear reference with a threshold of a minimal radius and fluorescent intensity implemented to differentiate DAPI-labelled nuclei against cell debris or non-specific binding. A watershed command in the R script was used to ensure segmentation and identification of nuclei which may be tightly clustered as distinct objects. The nuclei were dilated using kernel expansion to designate the soma region surrounding the nucleus. A distance map was generated for each image which calculates the distance of each foreground pixel (white) to the nearest background pixel (black). Protein expression was measured as the average pixel fluorescence intensity for each cell in each channel. In all experiments cells were stained with an antibody for NeuN, a protein specifically expressed in neurons, to localise protein changes to the neuronal or non-neuronal cellular populations in the culture. Proteins assessed by ICC were 5-HT_{1A}R (FIG. 7A), 5-HT_{2A}R (FIG. 7B).

Structural Experiments—we designed an experiment to assess whether cocaine, 5-MeO-DMT or their combination caused structural plasticity in cultured hippocampal neurons. The treatment groups were control, 24-hour 5-MeO-DMT (20 μM), 24-hour cocaine (25 μM) and 24-hour cocaine+24-hour 5-Meo. Primary hippocampal cultures were grown on 18 mm glass coverslips at a density of 1×10⁶ cells as described. On 3 DIV all cells underwent a full medium change. Cells in the combination group were treated with 2 μM cocaine for 24 hours, all other treatment groups remained in drug-free media. On 4 DIV, all cells underwent a second full medium change. Cells in the combination group were treated with 2 μM 5-MeO-DMT for a further 24 hours with all other groups beginning a 24-hour treatment. After treatment was completed the cells were fixed and processed as described below.

The design of this experiment was used for a series of antagonist studies to determine the essential signaling components of neuronal structural change mediated by 5-MeO-DMT or cocaine. Based on the findings with other psychedelic drugs that share structural and behavioral similarities to 5-MeO-DMT we selected ANA-12 (Sigma), an antagonist of TrkB, the BDNF receptor, and rapamycin (ThermoFischer) an antagonist of the mammalian target of rapamycin (mTOR). Due to the affinity and known behavioral change induced by 5-MeO-DMT at the 5-HT_{1A}R and 5-HT_{2A}R we assessed their contribution to structural changes mediated by 5-MeO-DMT. The receptors were antagonized with WAY-100635 maleate (Tocris) or Ketanserin (Axxora), respectively. For all antagonist studies the antagonist under investigation was added 15 mins prior to drug treatment. Antagonist concentrations were selected based on comparable published studies; 10 μM ANA-12, 100 nM rapamycin, 100 nM WAY-100635 and 100 μM Ketanserin. ANA-12, Ketanserin and rapamycin were dissolved in dimethyl sulfoxide (DMSO) with a final concentration of 0.01-0.04% for treatment. WAY100635 was dissolved in sterile dH₂O.

After termination of drug treatment coverslips were processed and imaged as outlined for immunocytochemistry. In all instances NeuN antibody staining was used to visualize projections from the cell body. Images were analyzed using the Simple Neurite Tracer and Sholl analysis plug-ins for ImageJ Fiji. For the Sholl analysis circle radii of 2 μm increments were used. All images were taken and analyzed by an experimenter blinded to treatment conditions.

Statistical Analysis—Data are represented as mean±SEM. Statistical analyses were performed using GraphPad Prism (version 5). For analyses involving comparison of three or more groups, a one-way ANOVA with Bonferroni's post-hoc test was utilized where appropriate. No statistics were calculated for the individual points of the Sholl plots, instead, statistical analyses were performed on the aggregate data, the area under the curve (AUC) of the Sholl plot. Secondary measurements were the Nmax, the maximum number of intersections measured by the Sholl analysis, which indicated the number of projections emanating from a neuron and the average length of primary neurites originating from the cell soma.

### Example 5—Chronic Cocaine±5-Meo-DMT: Behavior Study

### Cohort 1

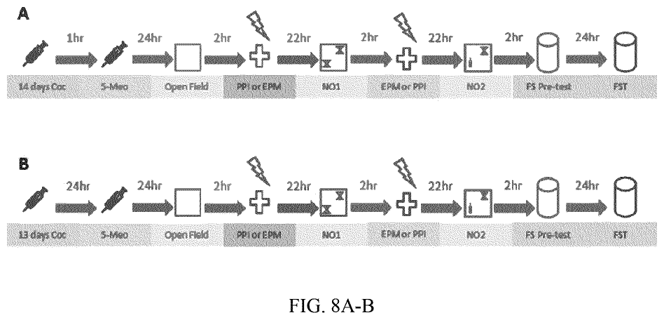

The experimental design was as described for the cocaine±5-MeO-DMT study. Animals received a daily i.p. injection of saline or cocaine (15 mg/kg for days 1-7 and 20 mg/kg for days 8-14) for 14 days followed by a single administration of 5-MeO-DMT 1 hour after the final saline or cocaine injection. In this instance the animals were 9 weeks old at the initiation of drug exposures and we selected just the high (20 mg/kg) dose of 5-MeO-DMT from the molecular study. Treatment groups for the study were saline+vehicle, saline+5-MeO-DMT, cocaine+vehicle and cocaine+5-MeO-DMT with n=16 for each group. Due to the number of behavioral paradigms to be completed by each animal we divided the study into two cohorts staggered by two weeks with 8 animals coming from each cohort to make up the 16 animals for each treatment group. 23 hours after 5-MeO-DMT administration the animals were introduced to the open field arena where they were allowed to freely explore the area for 5 mins. Following the open field test (OFT) animals were returned to their home cage for 2 hours before beginning either the elevated plus maze (EPM) or pre-pulse inhibition (PPI) testing. 24 hours after completing the OFT animals were returned to the chamber for the first session in novel object (NO1) testing. The animals were returned to their home cage for 2 hours before beginning the EPM or PPI test. 24 hours after NO1 the animals returned to the open field chamber for the second session in novel object (N02) testing. After completing N02 animals were returned to their home cage for 2 hours before they underwent the forced swim test (FST) pre-test. 24 hours later the animals were returned to the FS cylinders for the FST. Upon completion of the FST all animals were euthanized. A timeline of this experimental cohort is described in FIG. 8A.

In this instance, 5-MeO-DMT administration had strong adverse effects in a significant proportion of animals. Within 5-10 mins of administration a series of symptoms emerged that we believed were indicative of acute serotonin-associated toxicity, usually termed “serotonin syndrome” as a result of excessive serotonergic activity (Haberzettl et al. 2013). These symptoms overlap with those induced by acute psychedelic administration including head twitches, tremor, hind limb abduction, Straub tail, head shaking, head weaving and flat/low body posture however in this instance symptoms progressed rapidly to include hyperthermia, tachycardia, trembling and seizures. One animal died following a seizure and therefore to ensure animal welfare a number of animals that exhibited symptoms of excessive serotonin activation were euthanised. In total 7 of the 16 animals that were administered 20 mg/kg 5-MeO-DMT died or were euthanized for ethical reasons. 2 of these animals were in the saline+5-MeO-DMT group and 5 were in cocaine+5-MeO-DMT group. As the study was divided into two cohorts we consulted with the designated vet in BMF as to what amendments should be made to our study protocol to reduce the risk of harm to animals in cohort 2 and a number of changes were implemented to the study design as outlined below.

### Cohort 2

The experiment was conducted as described for cohort 1 for the first 13 days of injections. There are a number of notable differences in experimental procedures between the two cohorts of animals to be highlighted. Animals in cohort 2 were administered the higher dose of cocaine (20 mg/kg) for 6 days instead of 7, with cocaine exposures totaling 13 instead of 14 independent administrations. There was an interval of 24 hours following last cocaine before animals were administered 5-MeO-DMT instead of a 1-hour interval. Finally, the dose of 5-MeO-DMT was reduced to 10 mg/kg. The order and timing of all the behavioral paradigms were kept constant between the cohorts. A timeline of this experimental cohort is described in FIG. 8B. Additionally, 5-MeO-DMT administrations were staggered so animals could be observed for the duration of acute drug effects and to ensure recovery prior to the next animal being injected. The typical symptoms of psychedelic drug administration outlined above were reproduced in all animals administered 5-MeO-DMT. In this instance 2 animals were euthanized from the saline+5-MeO-DMT group as they exhibited early signs of serotonin-associated toxicity. Treatment groups arising from this cohort were as follows; saline+vehicle, saline+10 mg/kg 5-MeO-DMT, cocaine+vehicle and cocaine+10 mg/kg 5-MeO-DMT.

### Anxiety Behaviors

Anxiety is defined as a negative emotional state associated with the perception of a potential or ambiguous threat. It is characterized by apprehension, uncertainty, worries, uneasiness or tension stemming from the anticipation of potential threat or negative outcomes (Öhman, A. (2008) Fear and anxiety. Handbook of emotions, 709-729). The natural aversion exhibited by animals in different behavioral paradigms is used as an indicator of anxiety in animals. It assumes that anxiety involves a conflict between the drive to avoid and the drive to explore a perceived threatening stimulus with the tests juxtapositioning these conflicting drives (Crawley, J. N. (1985) Exploratory behavior models of anxiety in mice. Neuroscience & Biobehavioral Reviews, 9, 37-44., Salum, C., A. C. Roque-da-Silva & S. Morato (2003) Conflict as a determinant of rat behavior in three types of elevated plus-maze. Behavioural Processes, 63, 87-93). There is a high comorbidity of anxiety and drug addiction, and both can be precipitated through an inability to cope with persistent chronic stress. Stress is a powerful trigger of relapse to drug-taking behaviours through the activation of brain circuits involved in reward processing and in the attentional and mnemonic bias for drug use reminders (Duncan Md, E., W. Boshoven Bs, K. Harenski Bs, A. Fiallos Ms, H. Tracy Bs, T. Jovanovic PhD, X. Hu PhD, K. Drexler Md & C. Kilts PhD (2007) An fMRI Study of the Interaction of Stress and Cocaine Cues on Cocaine Craving in Cocaine-Dependent Men. The American Journal on Addictions, 16, 174-182). Therefore, our aim was to assess whether pharmacological intervention with 5-MeO-DMT can alleviate anxiety after the acute drug effects have subsided. To do this, two assessments of anxiety behavior, the open field test and the elevated plus maze were chosen.

### Open Field

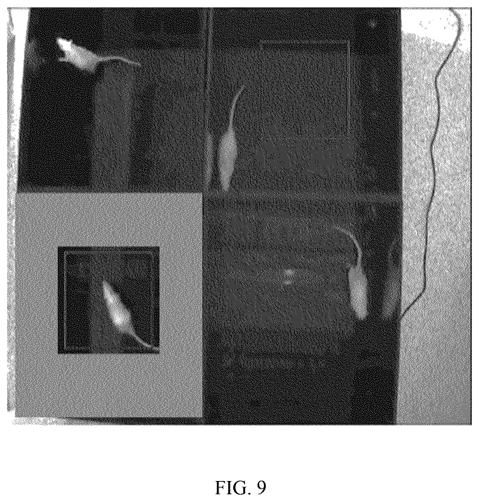

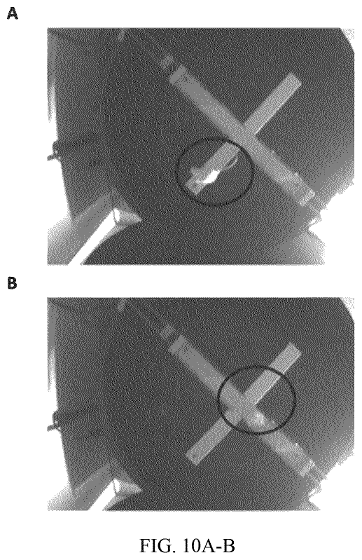

The open field environment provides a fast and relatively easy test that determines a variety of behavioral information ranging from general ambulatory ability to data regarding the emotionality of the subject animal. The technique enables investigation of different pharmacological compounds for anxiolytic or anxiogenic effects (Seibenhener, M. L. & M. C. Wooten (2015) Use of the Open Field Maze to Measure Locomotor and Anxiety-like Behavior in Mice. Journal of Visualized Experiments: JoVE, 52434). Rodents show distinct aversions to large, brightly lit, open and unknown environments (Choleris, E., A. W. Thomas, M. Kavaliers & F. S. Prato (2001) A detailed ethological analysis of the mouse open field test: effects of diazepam, chlordiazepoxide and an extremely low frequency pulsed magnetic field. Neuroscience & Biobehavioral Reviews, 25, 235-260). These features are incorporated in the open field test and form the basis of its use in behavioural paradigm testing. There are various parameters that can be quantified from the open field, but the primary parameter of interest is thigmotaxis, the tendency of a subject to remain close to walls as determined by measuring time spent in inner zones versus outer zones of the maze (Simon, P., R. Dupuis & J. Costentin (1994) Thigmotaxis as an index of anxiety in mice. Influence of dopaminergic transmissions. Behavioural brain research, 61, 59-64). Highly anxious animals exhibit greater thigmotaxis and vice versa. For the OFT, animals were placed in the open field arena and allowed to freely explore for 5 minutes. The session was recorded and analysed by EthoVision software which quantified the total distance covered. The number of entries into and total time spent in the centre of the arena were scored by two observers blind to experimental conditions (FIG. 9).

### Elevated Plus Maze

The EPM is one of the primary paradigms for the study of the neurobiological basis of anxiety and the screening for novel targets and anxiolytic compounds (Ennaceur, A. (2014) Tests of unconditioned anxiety Pitfalls and disappointments. Physiology & Behavior, 135, 55-71). It consists of four arms radiating from a central platform forming a plus sign shape; it is elevated from the ground with two opposed walled arms and two opposed open arms. The maze is based on the construct that preference for the enclosed arms is due to the greater aversiveness of the open arms. The paradigm utilizes similar animal characteristics as the open field test, accounting for an animal's innate aversion to large, brightly lit, open and unknown environments (Choleris, E., A. W. Thomas, M. Kavaliers & F. S. Prato (2001) A detailed ethological analysis of the mouse open field test: effects of diazepam, chlordiazepoxide and an extremely low frequency pulsed magnetic field. Neuroscience & Biobehavioral Reviews, 25, 235-260). Parameters that can be measured in the test are the number of entries into the open and closed arms of the maze (or ratio of open arm/total entries), the duration of time spent in the open arms of the maze and total ambulatory distance. Anxiogenic compounds accentuate the animal's natural aversion for the threatening but novel environment while anxiolytic compounds alleviate the aversion of the open arms. Total ambulatory distance is required to ensure that differential drug-induced behavior is attributable to effects on anxiety behaviors and not due to motor differences.

Our EPM apparatus consisted of a plus-shaped white plastic platform positioned 100 cm above the ground. Two opposite arms of the maze were bordered by vertical walls measuring 20 cm high, with the other two arms open to the environment with no edges. Animals were placed into the centre of the maze facing a closed arm and allowed to explore freely for 5 min. At the conclusion of the test, rats were returned to their home cages and the apparatus was cleaned with 2% distal. Animal movement was recorded during the trial using Any-Maze software. Time spent in open or closed arms, number of entries into open arms and number of grooming behaviors were all quantified (FIG. 10A-10B). Behavioral assessment was conducted in a quiet room under low-level red-light illumination. Behaviors were scored by two observers blind to experimental conditions.

### Sensory Processing: Pre-pulse Inhibition

Sensorimotor gating refers to the ability of a sensory event to suppress a motor response. Pre-pulse inhibition of startle (PPI) is an operational measure of sensorimotor gating that is studied across species as a basic feature of information processing, a means to understanding the basis of gating deficits in brain disorders, and a model for drug development (Swerdlow, N. R. & L. R. Squire. 2009. Prepulse Inhibition of Startle in Humans and Laboratory Models. In Encyclopedia of Neuroscience, 947-955. Oxford: Academic Press). The startle reflex consists of involuntary contractions of whole-body musculature elicited by sufficiently sudden and intense stimuli. The acoustic startle response is characterised by an exaggerated flinching response to an unexpected strong auditory stimulus (Ioannidou, C., G. Marsicano & A. Busquets-Garcia (2018) Assessing Prepulse Inhibition of Startle in Mice. Bio-protocol, 8, e2789 C1—Bio-protocol 2018; 8:e2789). PPI is characterised by a normal reduction in startle reflex that occurs when an intense startling stimulus is preceded by a brief low-intensity pre-pulse. Impaired PPI is observed in psychiatric disorders including schizophrenia, bipolar disorder, obsessive compulsive disorder and Tourette's syndrome. In patients with psychotic disorders, deficits in sensorimotor gating are associated with cognitive fragmentation and psychotic symptoms (Kapur, S. (2003) Psychosis as a State of Aberrant Salience: A Framework Linking Biology, Phenomenology, and Pharmacology in Schizophrenia. American Journal of Psychiatry, 160, 13-23). Cocaine withdrawal is often accompanied by psychotic symptoms (Tang, Y., N. L. Martin & R. O. Cotes (2014) Cocaine-Induced Psychotic Disorders: Presentation, Mechanism, and Management. Journal of Dual Diagnosis, 10, 98-106). Psychedelic drugs can inhibit PPI in the short term and are sometimes used to generate schizophrenia models in animals. Our aim was to assess whether withdrawal from repeated cocaine exposure, or acute psychedelic drug administration have a prolonged effect on PPI.

The PPI protocol employed was based on a procedure previously described by Geyer and colleagues (Geyer, M. A., K. Krebs-Thomson, D. L. Braff & N. R. Swerdlow (2001) Pharmacological studies of prepulse inhibition models of sensorimotor gating deficits in schizophrenia: a decade in review. Psychopharmnacology, 156, 117-154). Each rat was restrained in an appropriately sized cylindrical holder that was placed on a movement-sensitive platform and maintained in a soundproof chamber. The animal was allowed to habituate to a white noise background of 70 dB for 5 mins before receiving 5 20 ms startle trials of 120 dB separated by randomized intervals of 10-20 s. Immediately thereafter, each rat received 5 separate presentations with one of the pre-pulse stimuli of 72, 76, 80, or 84 dB followed 100 ms later by the 120 dB acoustic startle stimulus. Each trial was separated by a time interval of 10-20 s. The 4 pre-pulse stimuli were delivered in a randomised manner and included periods in which there was no pre-pulse or startle stimulus. The session terminated with five further startle trials. Randomisation of the sound signals delivered and quantification of the startle movements were recorded from the movement-sensitive platform. Signals were integrated using the software supplied by the manufacturers of equipment hardware (MED-Associates Inc., St. Albans, VT, USA).

### Cognitive Deficits: Novel Object Recognition

Human subjects with cocaine addiction show impaired performance in tasks involving attention, cognitive flexibility, and delayed reward discounting that are mediated by the medial and orbital prefrontal cortices, as well as spatial, verbal, and recognition memory impairments that are mediated by the hippocampus, and these deficits can predict poor treatment outcomes (Bolla, K. I., D. A. Eldreth, E. D. London, K. A. Kiehl, M. Mouratidis, C. Contoreggi, J. A. Matochik, V. Kurian, J. L. Cadet, A. S. Kimes, F. R. Funderburk & M. Ernst (2003) Orbitofrontal cortex dysfunction in abstinent cocaine abusers performing a decision-making task. Neurolmage, 19, 1085-1094; Aharonovich, E., D. S. Hasin, A. C. Brooks, X. Liu, A. Bisaga & E. V. Nunes (2006) Cognitive deficits predict low treatment retention in cocaine dependent patients. Drug and Alcohol Dependence, 81, 313-322).

The novel object recognition (NOR) task is a widely used model for investigation into memory alterations (Antunes, M. & G. Biala (2012) The novel object recognition memory: neurobiology, test procedure, and its modifications. Cognitive processing, 13, 93-110). The task is a simple behavioural assay of memory that relies primarily on a rodent's innate exploratory behaviour in the absence of externally applied rules or reinforcement. The objective is to assess an animal's behaviour when it is exposed to two objects, a novel and a familiar object, given the animal's natural propensity to investigate novelty (Baxter, M. G. (2010) “I've seen it all before”: Explaining age-related impairments in object recognition. Theoretical comment on Burke et al. (2010)). The test has been used to investigate the effects of various pharmacological treatments or brain manipulations on memory (Goulart, B. K., M. N. M. De Lima, C. B. De Farias, G. K. Reolon, V. R. Almeida, J. Quevedo, F. Kapczinski, N. Schröder & R. Roesler (2010) Ketamine impairs recognition memory consolidation and prevents learning-induced increase in hippocampal brain-derived neurotrophic factor levels. Neuroscience, 167, 969-973). Object recognition is measured by the difference in the exploration time of novel and familiar objects, sometimes displayed as a discrimination index. The recognition measure is influenced by the interval between time spent with the familiar and the novel object and the duration of time the animal is allowed to explore the familiar object in the first trial. The preference for a novel object means that presentation of the familiar object exists in the animal's memory (Ennaceur, A. (2010) One-trial object recognition in rats and mice: methodological and theoretical issues. Behavioural brain research, 215, 244-254). Pharmacological interventions and circuit level remodeling may alter this bias indicating alterations in some aspect of memory processing, recognition, acquisition, or storage.

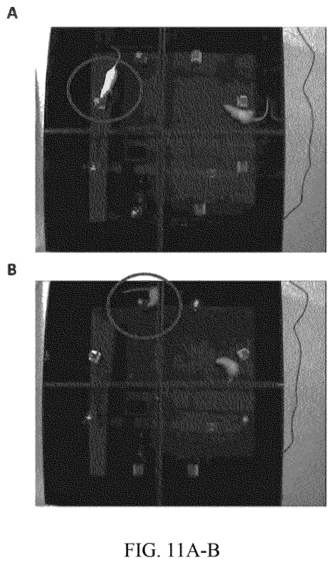

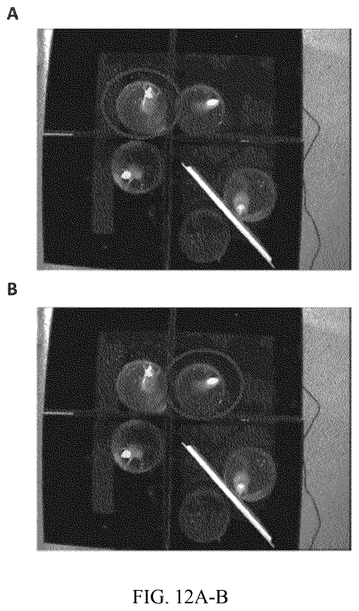

Animals were placed into the chamber with two identical objects during NO1 and allowed to freely explore the chamber and interact with the objects for 10 minutes. After 24 hours animals were returned to the chamber with one of the objects being replaced with a novel object, animals were again allowed to freely explore the chamber and interact with the objects for 10 mins. The sessions were recorded and interaction with the novel or familiar objects was measured by an observer blind to experimental conditions (FIG. 11A-11B). Between trials the chambers and objects were cleaned with 2% distal. Behaviors were scored by two observers blind to experimental conditions.

### Depressive Phenotype: Forced Swim Test

Addiction and depression are highly comorbid, with nearly one third of patients with major depressive disorder also having substance use disorders and comorbidity yielding higher risk of suicide and greater social and personal impairment (Davis, L., A. Uezato, J. M. Newell & E. Frazier (2008) Major depression and comorbid substance use disorders. Current opinion in psychiatry, 21, 14-18). Chronic maladaptations in the mesolimbic dopamine circuit and connected structures may underlie both addiction and depression, and changes in gene expression are likely to play a crucial role in these maladaptations (Gajewski, P. A., G. Turecki & A. J. Robison (2016) Differential Expression of FosB Proteins and Potential Target Genes in Select Brain Regions of Addiction and Depression Patients. PLoS ONE, 11, e0160355).

The forced swim test (FST) is one of the most commonly used behavioural assays to assess a depressive-like phenotype (Cryan, J. F., A. Markou & I. Lucki (2002) Assessing antidepressant activity in rodents: recent developments and future needs. Trends in pharmacological sciences, 23, 238-245). The test consists of an animal placed in a container filled with water from which it cannot escape. Initially the animal will struggle and swim in an attempt to escape before exhibiting immobility. Immobility in the context of the FST is defined as floating with the absence of any movement except for those necessary for keeping the nose above water (Yankelevitch-Yahav, R., M. Franko, A. Huly & R. Doron (2015) The Forced Swim Test as a Model of Depressive-like Behavior. Journal of Visualized Experiments: JoVE, 52587). The test is used to monitor depressive-like behaviour and is based on the assumption that immobility reflects a measure of behavioural despair (Cryan, J. F. & A. Holmes (2005) Model organisms: the ascent of mouse: advances in modelling human depression and anxiety. Nature reviews Drug discovery, 4, 775). The measured outcomes from the test are the time spent immobile and time participating in active behaviours which can be subdivided into swimming and climbing behaviours. Reductions in immobility are the primary measure for an antidepressive effect but there is often differential effects on swimming or climbing behaviour dependent on the mechanism of action of the drug under investigation (Cryan, J. F., R. J. Valentino & I. Lucki (2005) Assessing substrates underlying the behavioral effects of antidepressants using the modified rat forced swimming test. Neuroscience & Biobehavioral Reviews, 29, 547-569). Advantages of the FST as a behavioural model of depression include the depressive phenotype being precipitated by an exposure to an inescapable stress. Stress is a powerful trigger of relapse to drugtaking behaviours through the activation of brain circuits involved in reward processing (Duncan Md, E., W. Boshoven Bs, K. Harenski Bs, A. Fiallos Ms, H. Tracy Bs, T. Jovanovic PhD, X. Hu PhD, K. Drexler Md & C. Kilts PhD (2007) An fMRI Study of the Interaction of Stress and Cocaine Cues on Cocaine Craving in Cocaine-Dependent Men. The American Journal on Addictions, 16, 174-182). Similarly, a broad range of antidepressant drugs demonstrate efficacy in the test making it a suitable screening test for novel antidepressant drugs (Borsini, F. & A. Meli (1988) Is the forced swimming test a suitable model for revealing antidepressant activity?Psychopharmacology, 94, 147-160).

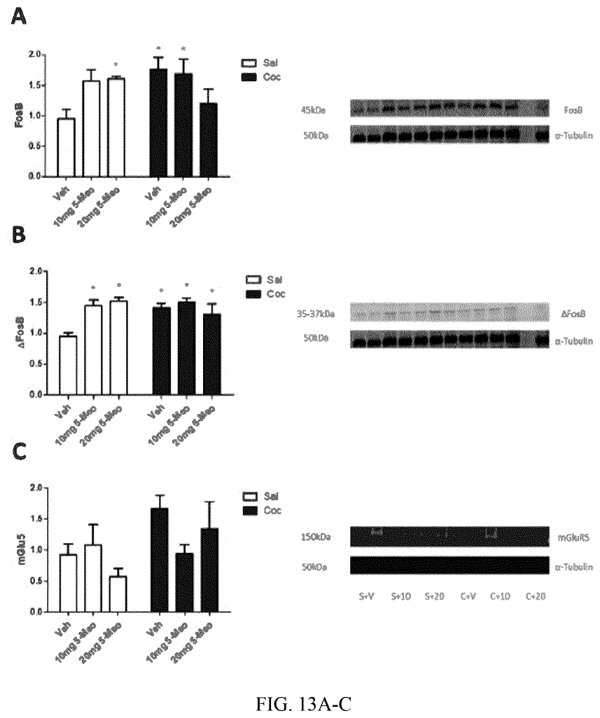

The FST apparatus consisted of a clear Plexiglas cylinder measuring 80 cm tall, 20 cm in diameter and filled with 40 cm of 24+1° C. water. The FST was conducted over two sessions on two consecutive days. During the first session animals were subjected to a pre-test phase in which they were placed in the cylinder for 15 min before being dried and returned to their home cage. 24 hours later, rats were again placed in the FST apparatus for 5 min and their activity was video recorded (FIG. 12A-12B). Each video was scored for immobility, swimming, and climbing behavior by trained blinded observers.

### Example 6—Molecular Changes Induced by Chronic Cocaine±5-MeO-DMT

Drugs of abuse, including cocaine, can induce robustly stable protein changes throughout the addiction circuitry. The first aim of the study was to determine whether 5-MeO-DMT could alter the expression of these proteins and if so, how this alteration was affected by the marker assessed, the brain region of interest and the dose of drug administered. While FosB, ΔFosB, CREB, mGluR5, G9a, 5-HT_{1A}R and BDNF were assessed in most if not all brain regions, only the significant changes are reported in the sections that follow.

### Nucleus Accumbens (NAc)

In the NAc repeated cocaine increases the expression of FosB and ΔFosB at 24 hrs post last exposure (FIG. 13A-13B). For both proteins basal expression levels were re-established at 120 hr post last cocaine. There is a dose-dependent regulation of FosB by 5-MeO-DMT, with 20 mg/kg but not 10 mg/kg increasing expression. Alternatively, ΔFosB is increased by both 10 mg/kg and 20 mg/kg 5-MeO-DMT. This 5-MeO-DMT-mediated increase in FosB and ΔFosB does not persist to 120 hours. Despite an overlapping pattern of regulation by cocaine or 5-MeO-DMT, the combination of cocaine and 20 mg/kg 5-MeO-DMT does not have an additive effect. In both instances there is a significant interaction between cocaine and 5-MeO-DMT (p=0.0110 and p=0.0051) with the combo group showing no significant difference in FosB expression levels relative to the saline+vehicle control group. This effect does not extend to ΔFosB where expression levels remain elevated in the combo group relative to control.

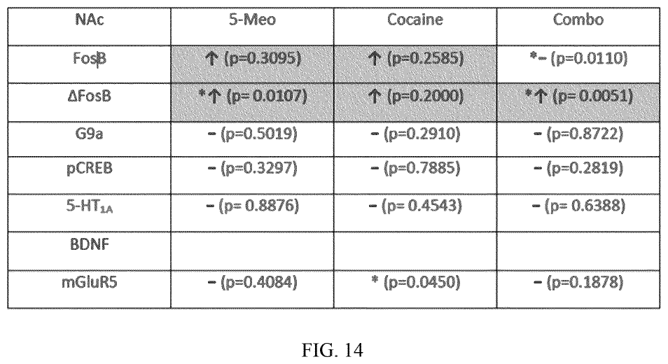

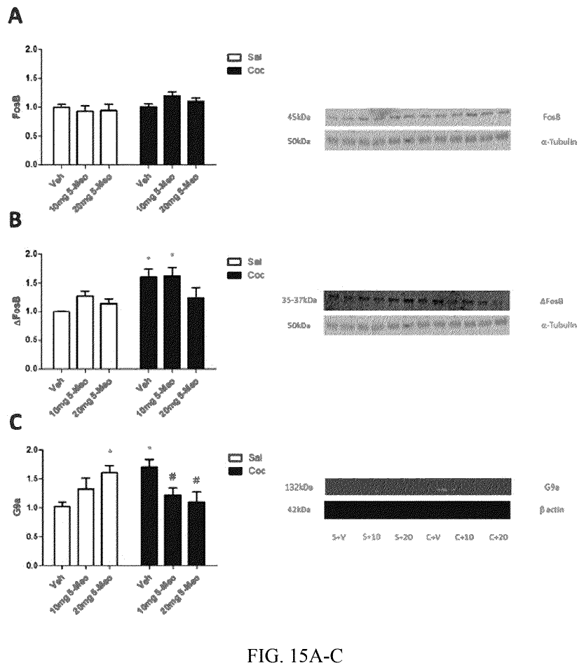

There is a significant cocaine effect on mGluR5 expression (FIG. 13C, p=0.0450) in the NAc determined by two-way ANOVA, however this effect does not survive post hoc comparison. None of the other tested markers show regulation in expression in the NAc by cocaine, 5-MeO-DMT or the combination at either timepoint measured. The findings from the NAc are summarized in FIG. 14.

### Dorsal Striatum

The dorsal striatum (DS) is a key locus of adaptation following sustained cocaine exposure as drug consumption shifts from impulsive to compulsive use. Repeated cocaine has a significant effect on FosB (FIG. 15A, p=0.0217) at 24 hours as determined by two-way ANOVA, with no post hoc differences between treatment groups. ΔFosB expression values failed the Bartlett's and Levene's test for equal variance and therefore could not be analysed by the parametric two-way ANOVA, instead the data was analyzed by the non-parametric Kruskal-Wallis test with Dunn's post hoc testing which found a significant increase in ΔFosB expression in the cocaine+vehicle and cocaine+10 mg/kg 5-MeO-DMT treatment groups relative to control (FIG. 15B).

Contrasting with the finding in the NAc 5-MeO-DMT alone does not regulate the expression of either form of the protein. However, 20 mg/kg 5-MeO-DMT returns the cocaine-induced increase in ΔFosB to levels comparable with control. As with the NAc there is no change in expression levels of either form of the protein at 120 hours irrespective of the treatment group.

Cocaine increased the expression of the G9a protein at 24 hours (FIG. 15C), with no change present at 120 hours. 20 mg/kg 5-MeO-DMT increased G9a expression levels at 24 hours which remained significantly elevated at 120 hours. The addition of either concentration of 5-MeO-DMT to cocaine exposure significantly reduced G9a levels relative to the cocaine+vehicle group resulting in expression levels comparable to control.

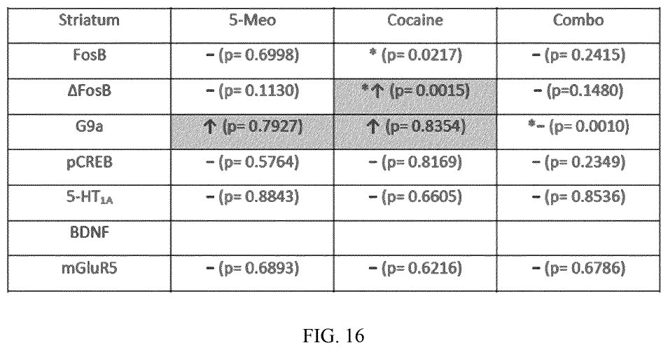

The other protein markers assessed in the DS remained unchanged by any of the treatment groups at 24 hours. The findings from the DS are summarized in FIG. 16.

### PFC

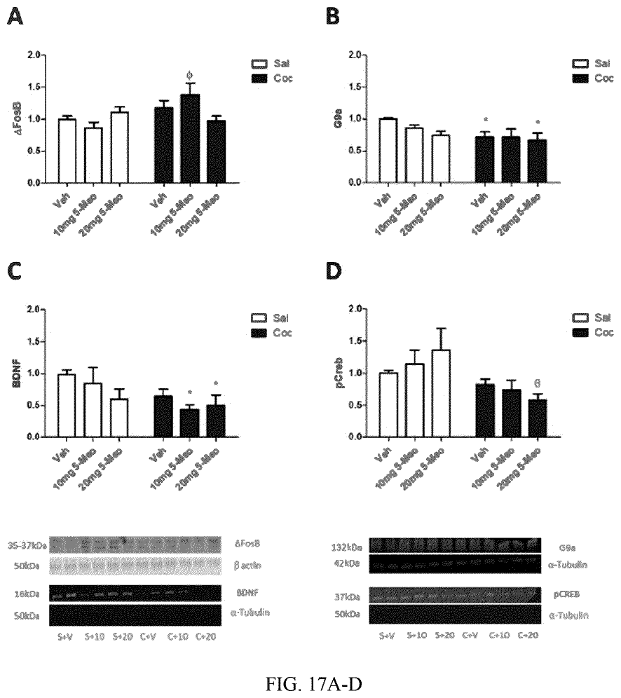

In the PFC FosB is unchanged by any of the treatments at either timepoint. At 24 hours there is a significant cocaine effect (p=0.0411) and a significant interaction between cocaine and 5-MeO-DMT (p=0.0205) on ΔFosB expression. Neither treatment changes expression alone but the combination of cocaine coupled with 10 mg/kg 5-MeO-DMT increases expression relative to 10 mg/kg 5-MeO-DMT alone (FIG. 17A). This effect appears to be dose dependent as it is not replicated in the cocaine+20 mg/kg 5-Meo. At 120 hours there is a significant cocaine effect (p=0.0144) on ΔFosB expression determined by two-way ANOVA but with no differences between treatment groups found by post hoc testing.

At 24 hours G9a expression values failed the test for normality (kolmogorov smirnov test) and as there is no non-parametric equivalent of the two-way ANOVA the data was analysed by the non-parametric Kruskal-Wallis test followed by Dunn's post hoc test. There was a significant decrease in G9a expression in the cocaine+vehicle and the cocaine+20 mg/kg 5-MeO-DMT groups relative to the saline+vehicle control groups (FIG. 17B). At 120 hours G9a expression values display normal distribution and were therefore analysed by two-way ANOVA. Cocaine significantly affects expression levels (p=0.0187) however there were no significant differences between treatment groups determined by post hoc test.

Similarly, at 24 hours BDNF expression values failed the test for normality and therefore the data was analyzed by the Kruskal-Wallis test followed by Dunn's post hoc test. There was a significant decrease in BDNF expression in the cocaine+10 mg/kg 5-MeO-DMT and the cocaine+20 mg/kg 5-MeO-DMT groups relative to the saline+vehicle control groups (FIG. 17C).

There is no change in the proportion of pCREB relative to total CREB at either timepoint, however, cocaine has a significant effect on pCREB levels at 24 hours (FIG. 17D, p=0.0048) and 120 hours (p=0.0259). At 24 hours there is a decrease in pCREB in the cocaine+20 mg/kg 5-MeO-DMT group relative to saline+20 mg/kg group.

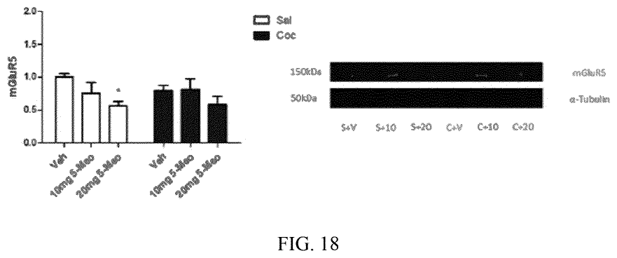

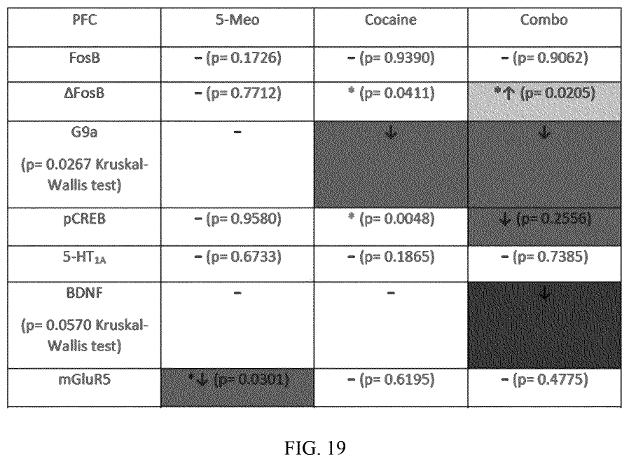

There is data showing 5-MeO-DMT treated cerebral organoids have a decrease in mGluR5 receptor expression, the authors suggest this effect may be useful clinically in the treatment of drug addiction due to the role these receptors play in NAc. There was no change in NAc mGluR5 expression following 5-MeO-DMT treatment with either tested concentration. However, in the PFC there is a significant 5-MeO-DMT effect on receptor expression levels (p=0.0301), with a significant decrease in receptor expression mediated by the 20 mg/kg 5-MeO-DMT in saline treated animals (FIG. 18). The findings from the PFC are summarized in FIG. 19.

### Amygdala

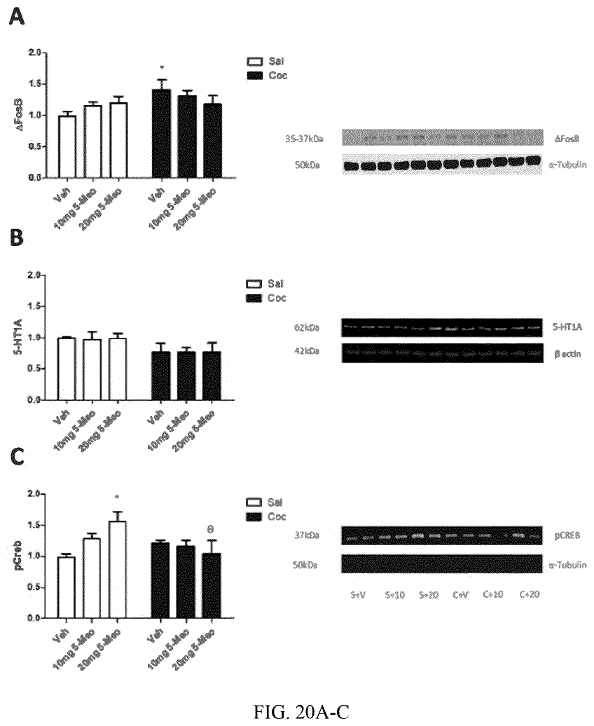

In the Amygdala there is no change in FosB levels at ₂₄ hours, at 120 hours there is a significant interaction between cocaine and 5-MeO-DMT (p=0.0353) with no post hoc differences between treatment groups. ΔFosB expression is significantly affected by cocaine at 24 hours (p=0.0463) with an increase in expression in the cocaine+vehicle group (FIG. 20A). There was no effect of 5-MeO-DMT alone on ΔFosB expression at this timepoint, however 20 mg/kg 5-MeO-DMT normalized levels in the cocaine treated animals comparable to the control group. Alternatively, at 120 hours there is no cocaine-induced increase in ΔFosB but there is a dose-dependent increase present in the 20 mg/kg 5-MeO-DMT group relative to control.

There is a significant cocaine effect (FIG. 20B, p=0.0172) on 5-HT_{1A}R expression at 24 hours with no post hoc differences between treatment groups. This cocaine effect does not persist to 120 hours.

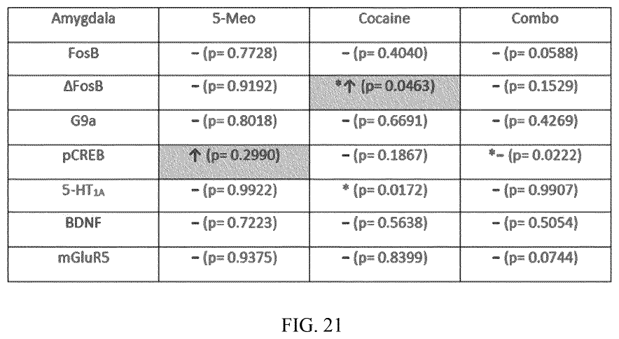

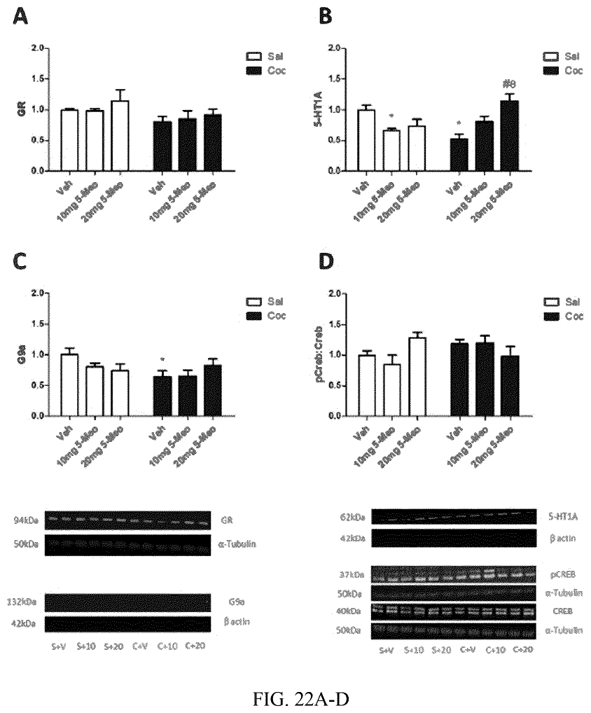

The proportion of pCREB was unchanged by any of the treatments at either timepoint, however, at 24 hours the levels of pCREB are increased by the 20 mg/kg 5-MeO-DMT, specifically in the saline-treated animals (FIG. 20C). There is a significant interaction between cocaine and 5-MeO-DMT affecting pCREB expression (p=0.0222) at 24 hours. The findings from the Amygdala are summarized in FIG. 21.

### Hippocampus

In the hippocampus there was no detected alteration in expression of either form of FosB protein.

The two-way ANOVA found cocaine significantly affected GR expression (FIG. 22A, p=0.0434) at 24 hours albeit with no post hoc differences present. This effect was not detectable at 120 hours, and there was no 5-Meo-associated regulation of GR at either timepoint.

5-MeO-DMT is a non-selective serotonin receptor agonist, given its high affinity for the 5-HT_{1A}R, the high expression levels of the receptor within the hippocampus and some evidence of repeated cocaine exposure altering receptor expression we assessed whether there was altered levels of the target receptor expression. Repeated cocaine decreased 5-HT_{1A}R expression at 24 hours (FIG. 22B). Both concentrations of 5-MeO-DMT alone show a trend towards decreasing expression levels but only the 10 mg/kg concentration achieves significance. There is a significant interaction between cocaine and 5-MeO-DMT (p<0.001) with either 5-MeO-DMT concentration restoring receptor expression to levels comparable to control. Receptor expression levels are equal across treatment groups at the 120-hour timepoint.

G9a expression was reduced by cocaine at 24 hours (FIG. 22C) with no change present at 120 hours. There is no change in enzyme expression mediated by 5-MeO-DMT alone, however, 20 mg/kg 5-MeO-DMT normalised the cocaine-induced decrease in G9a present at 24 hours.

There is no change in pCREB levels associated with either treatment but there is a significant interaction between treatments on the proportion of pCREB (FIG. 22D, p=0.0265) at 24 hours with no post hoc differences between groups.

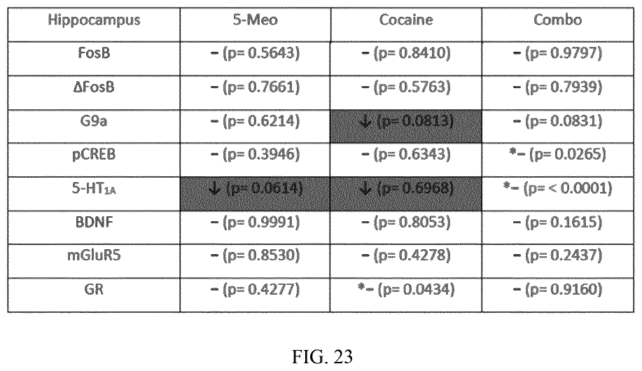

There was no change in BDNF expression at the 24-hour timepoint in any of the treatment groups. 120 hours post last cocaine there is a significant decrease in BDNF levels in both the cocaine+vehicle group and the 20 mg/kg 5-MeO-DMT group with a significant interaction between the two factors (p=0.0025). The 10 mg/kg 5-MeO-DMT group has no effect alone and does not affect the cocaine-induced decrease in expression levels. 20 mg/kg 5-MeO-DMT in the cocaine-experienced group restores BDNF levels to those seen in the control group. The findings from the hippocampus are summarized in FIG. 23.

### Example 7—Hippocampal Mass Spectrometry Proteomics Results

Having assessed a series of proteins known to be regulated by cocaine across the addiction circuitry on an individual basis we next wanted to implement a high throughput method to determine a comprehensive pattern of protein regulation after both repeated cocaine and 5-MeO-DMT exposure in a single brain region. The hippocampus was chosen to be assessed due to its contribution to the addicted phenotype and the abundance of target receptors for 5-MeO-DMT to mediate a measurable effect. Implementation of an unbiased proteomic assessment enables the identification of specific pathways being regulated by each treatment providing an explanatory context for understanding global treatment effects.

Tryptic peptide solutions of protein extracts from hippocampal tissue samples were subjected to LCMS/MS. The resulting MS spectra were searched against the rat UniProt database (Last modified June 2019). The search identified 2,717 proteins from the hippocampal tissue extracts. After filtering to remove identifications from the reverse database only identified by site and common contaminants the number was reduced to 2,681. This was further reduced to 1,847 when the search was filtered by valid values such that proteins that were not present in a minimum of 4 samples in at least one group were eliminated.

Animals that received daily saline and a vehicle treatment (saline+vehicle) were compared to animals in each individual treatment group to establish differentially expressed proteins characteristic of those treatment conditions with n=6 per treatment group. These comparisons were made using a Student's t-test, unadjusted, with a threshold p-value<0.05 coupled with a fold change of ≥1.2 or <−1.2. 340 proteins were found to be differentially expressed between the control and cocaine-experienced animals. This threshold for differential protein expression was deemed biologically relevant. Repeated cocaine has been shown to induce a 19% reduction in G9a expression and a 15% decrease in H3K9Me2 expression both of which alter addiction-linked behaviors including CPP, a measure of cocaine reward (Maze, I., H. E. Covington, D. M. Dietz, Q. LaPlant, W. Renthal, S. J. Russo, M. Mechanic, E. Mouzon, R. L. Neve, S. J. Haggarty, Y. Ren, S. C. Sampath, Y. L. Hurd, P. Greengard, A. Tarakhovsky, A. Schaefer & E. J. Nestler (2010) Essential Role of the Histone Methyltransferase G9a in Cocaineinduced Plasticity. Science (New York, N.Y.), 327, 213). 118 and 120 proteins were differentially expressed in the 10 mg/kg and 20 mg/kg 5-MeO-DMT groups, respectively. The combination of cocaine+10 mg/kg 5-MeO-DMT yielded 541 differentially expressed proteins while in the cocaine+20 mg/kg 5-MeO-DMT there were 221 differentially expressed proteins.

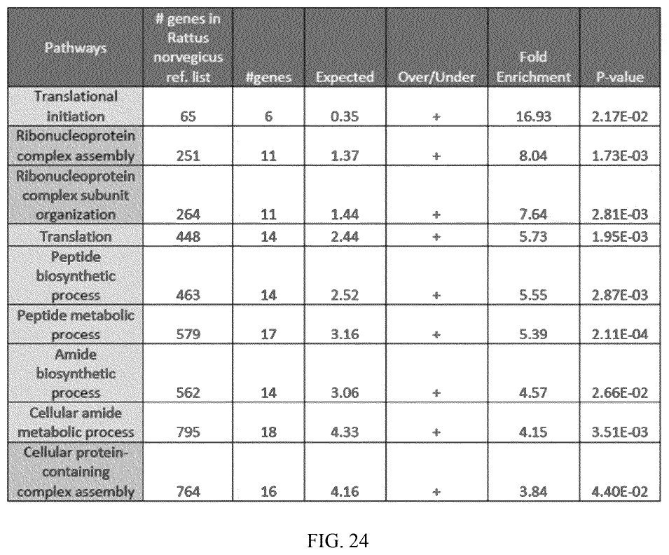

To identify pathways that were statistically over- or under-represented in each treatment group the list of proteins were examined for statistical overrepresentation with Fisher's exact test and Bonferroni's correction for multiple testing (p<0.05) using the online PANTHER classification system (version 14.1). This failed to map 25% of the differentially regulated proteins. Instead the analysis was rerun with the corresponding genes to the identified proteins, this reduced the unknown IDs to <1%. For each treatment group the list of genes was compared to the Rattus norvegicus genome. For the cocaine group there were 157 pathways significantly overrepresented. 16 and 24 pathways were significantly overrepresented in the 10 mg/kg and 20 mg/kg 5-MeO-DMT groups respectively. The combination of cocaine+10 mg/kg 5-MeO-DMT yielded 309 significantly overrepresented pathways while in the cocaine+20 mg/kg 5-MeO-DMT there were 67 significantly overrepresented pathways. In each instance the pathway list was condensed to the top 5-10 listed pathway for each treatment group ranked by fold enrichment (FIG. 24-28).

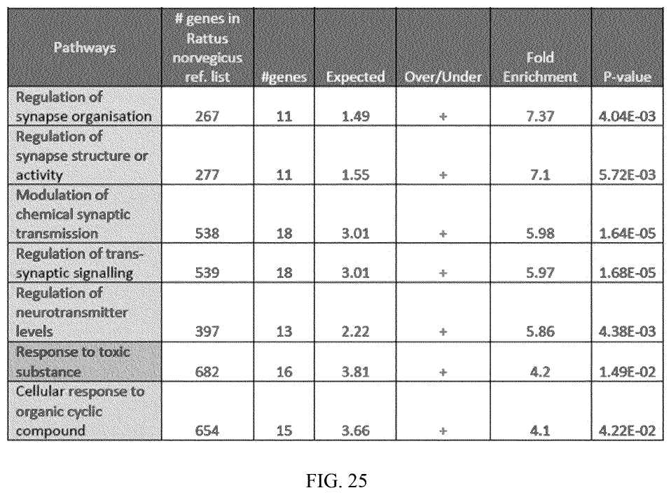

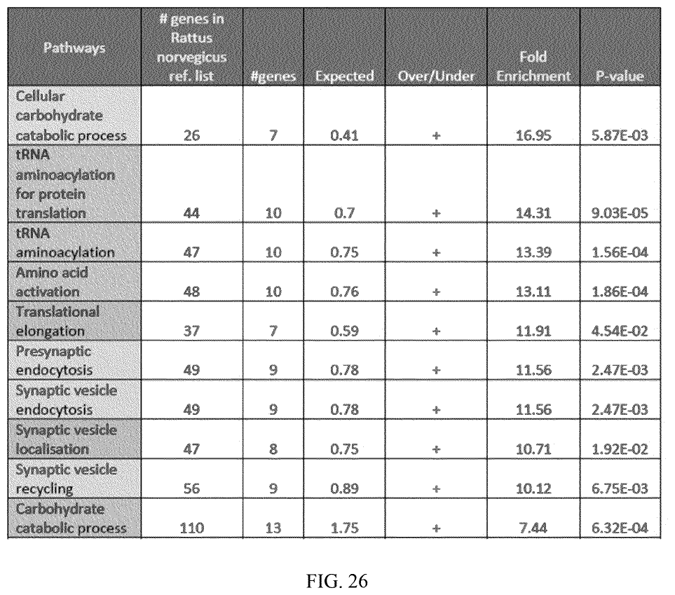

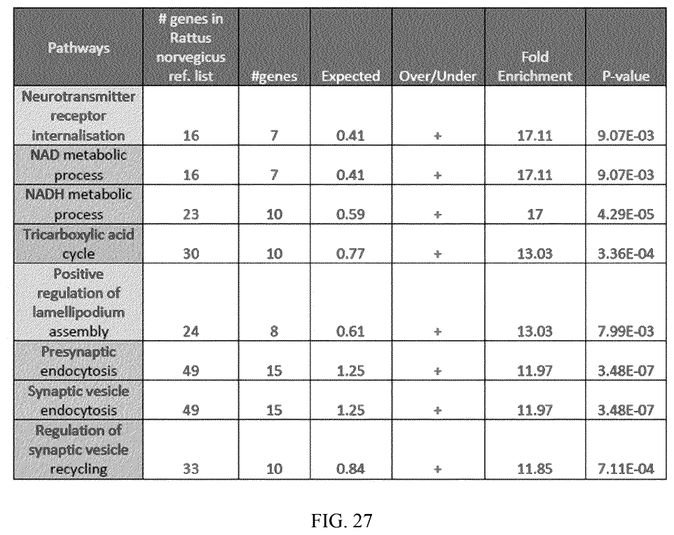

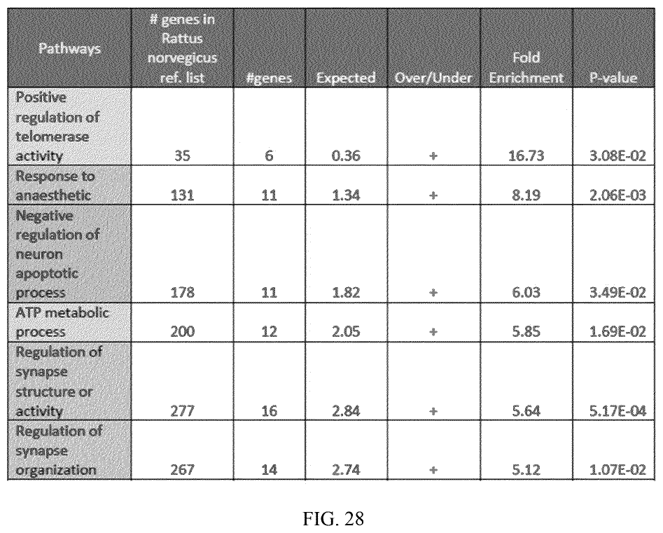

The top pathways overrepresented in the cocaine-experienced animals were involved in the regulation of metabolic processes, protein translation and synaptic signalling (FIG. 26). 10 mg/kg 5-MeO-DMT altered protein translation and synthesis (FIG. 24) while 20 mg/kg 5-MeO-DMT lead to the regulation of synapse organisation, structure and activity as well as modulating chemical transmission (FIG. 25). The combination of cocaine and 10 mg/kg 5-MeO-DMT altered the expression of pathways involved in NAD and NADH metabolism, positive regulation of lamellipodium assembly and modulation of synaptic signaling (FIG. 27). The top overrepresented pathways following cocaine+20 mg/kg 5-MeO-DMT were positive regulation of telomerase activity, negative regulation of apoptosis and regulation of synapse organization, structure and activity (FIG. 28).

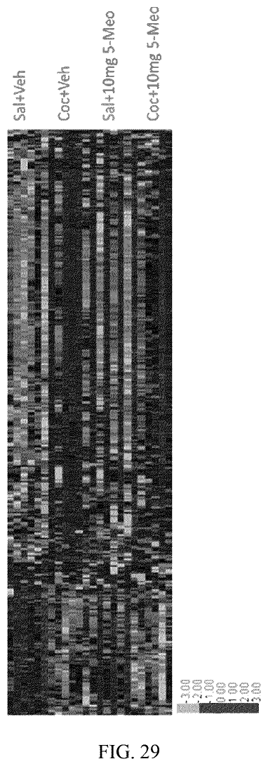

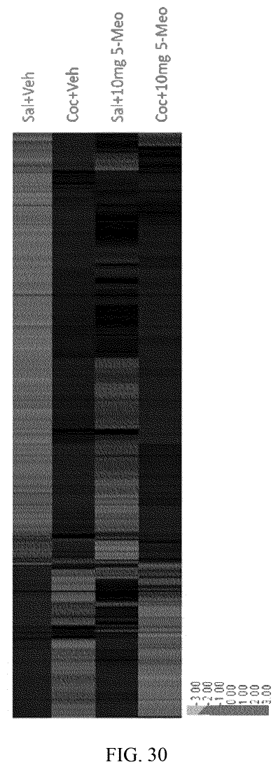

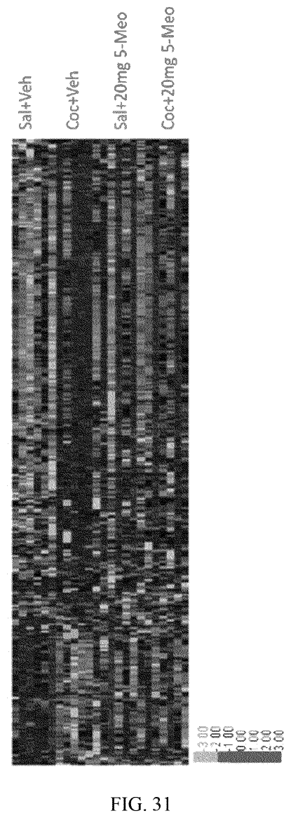

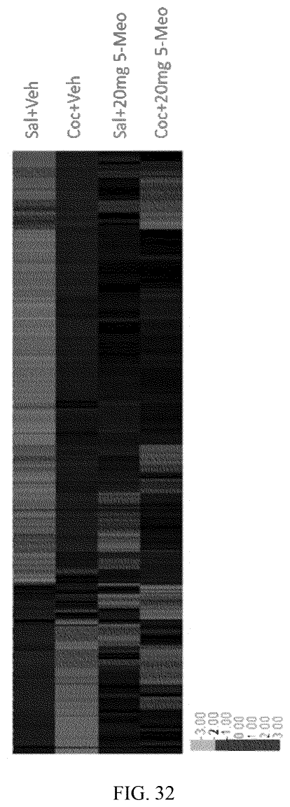

Differentially expressed proteins (again using Gene IDs) for each treatment group were entered into open-sourced clustering software Cluster 3.0 (version 1.58) for Hierarchical clustering. Dual clustering using Euclidean distance measures and average linkage arranged samples in columns and altered proteins in rows according to their Z-score. All values from each treatment group are clustered separately and clusters of upregulated and downregulated are generally consistent across groups, with some notable outliers. Proteins are arranged in heat maps for visualization purposes with control, cocaine, one dose of 5-MeO-DMT and the corresponding combination group were also generated (FIG. 29-32). The process of hierarchical clustering groups proteins together that appear to be regulated in a similar manner regarding the direction of change in expression and the magnitude of that change. The process does not group proteins together based on their biological function, grouping by function is done though pathway enrichment.

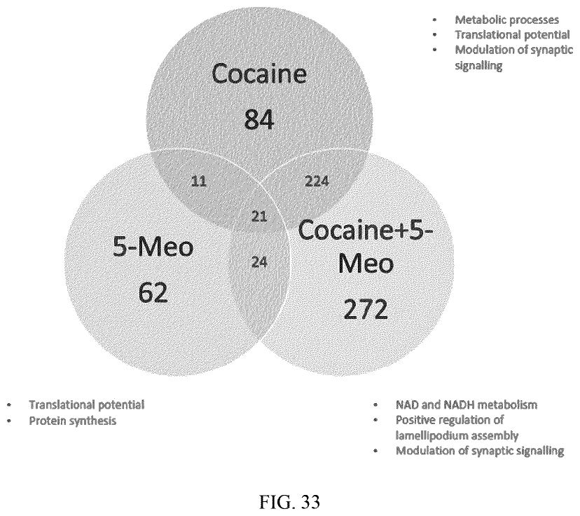

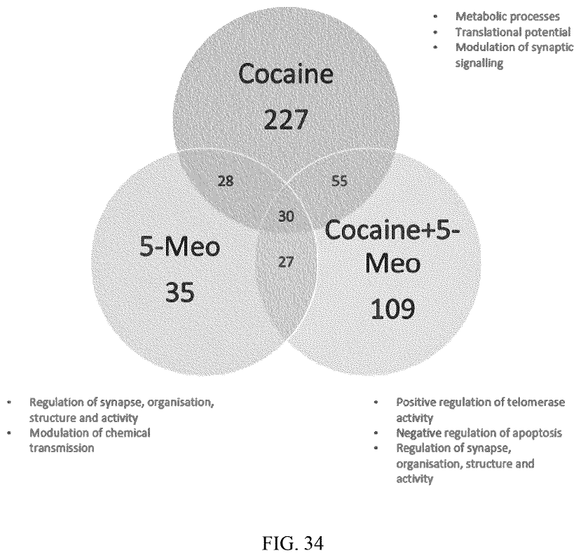

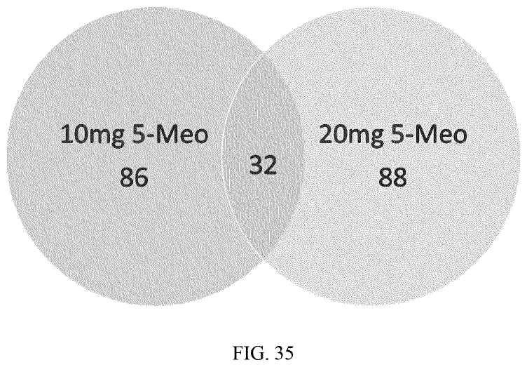

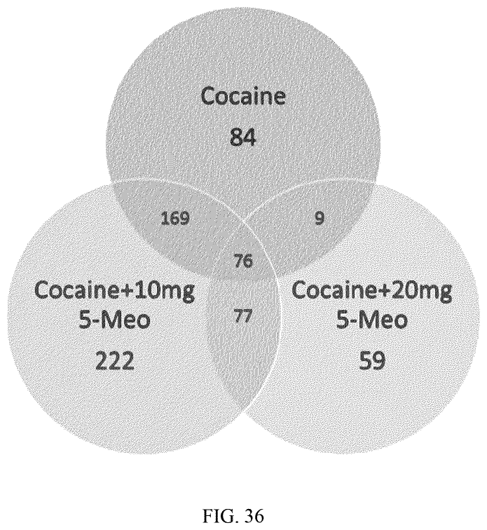

To facilitate overview comparisons between treatment groups the data has been arranged into Venn diagrams showing shared and alternative protein regulation (FIG. 33-36).

### Example 8—In Vitro Assessment of Cocaine and 5-MeO-DMT on Cultured Hippocampal Neuron Structure

Having implicated 5-MeO-DMT in regulating proteins associated with plasticity in vivo, we then wanted to determine whether we could replicate these changes in an in vitro model and subsequently use this model to study the mechanisms underpinning 5-Meo-associated plastic change. Many psychedelics have been demonstrated to induce structural change in cultured neurons through a mechanism of 5-HT_{2A}R activation, BDNF and mTOR signalling and given the shared pharmacology of 5-MeO-DMT with the other psychedelics we wanted to determine if this mechanism was conserved across compounds.

### Cocaine and 5-Meo

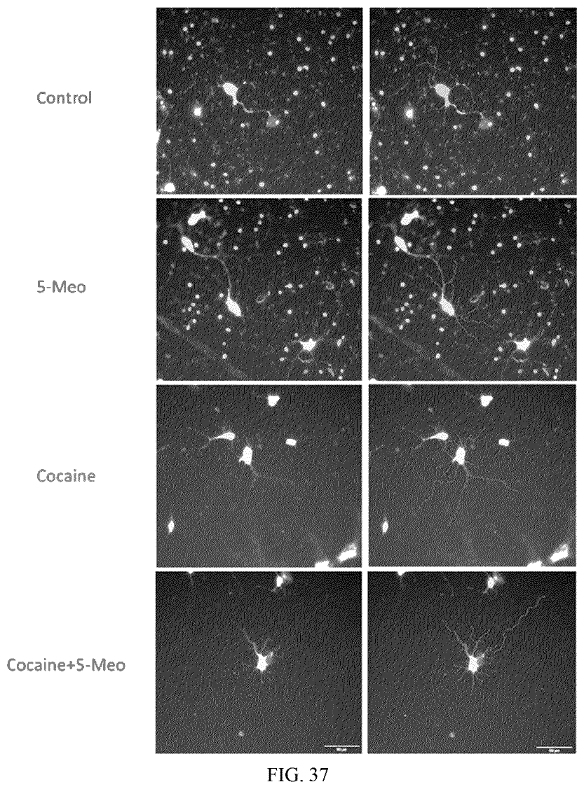

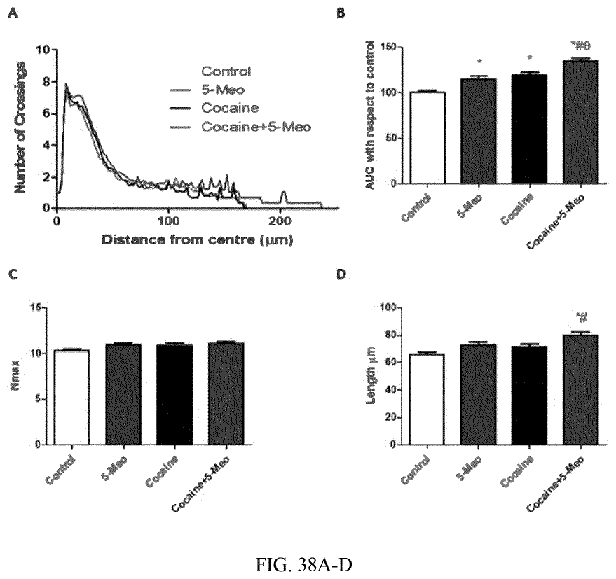

Representative neurons and their corresponding traces for each treatment group are shown in FIG. 37. Sholl plots (FIG. 38A) were generated for the traced neurons (n=169-180/group) in each treatment group by running a Sholl analysis. Each treatment group significantly increased the normalised area under curve (AUC) of the Sholl plot relative to control as determined by one-way ANOVA with Bonferroni's post hoc test (FIG. 38B). The combination of cocaine+5-MeO-DMT also increased the normalised AUC relative to either compound alone (FIG. 38B). The maximum number of intersections counted were unchanged between treatment groups (FIG. 38C). The combination group significantly increased the average neurite length relative to both the control and cocaine alone treatment groups (FIG. 38D).

### Cocaine and 5-MeO-DMT±WAY100635

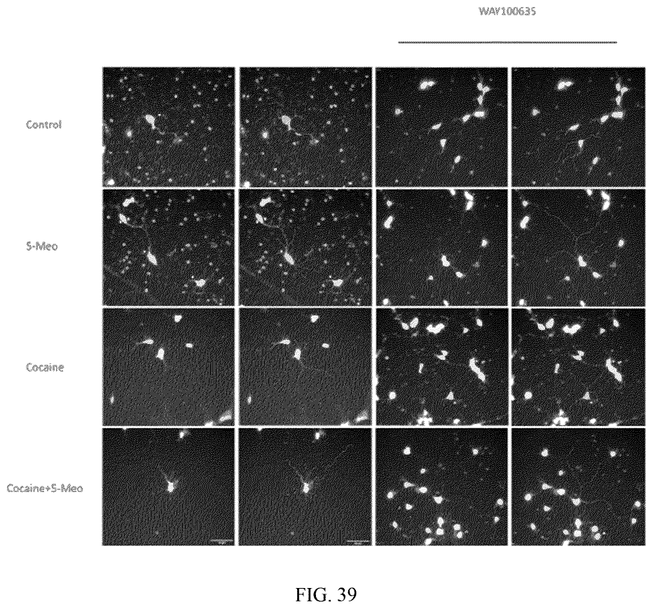

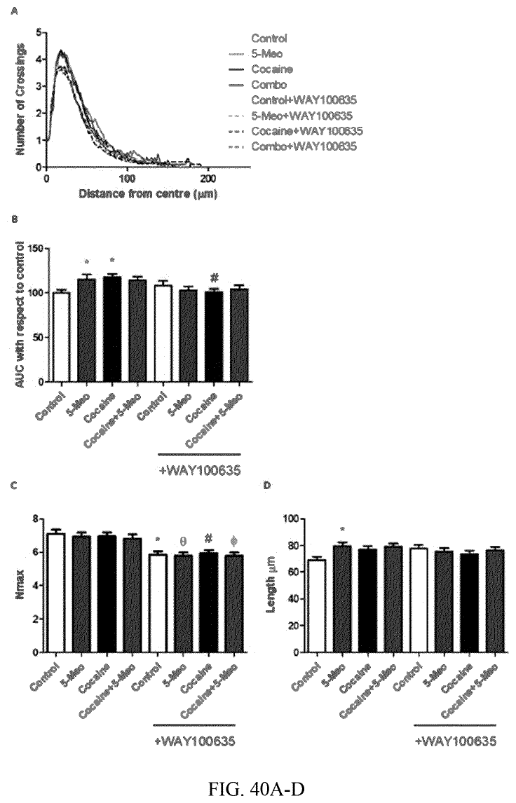

Representative neurons and their corresponding traces for each treatment group are shown in FIG. 39 Sholl plots (FIG. 40A) were generated for the traced neurons (n=110-180/group) in each treatment group by running a Sholl analysis. 5-MeO-DMT and cocaine independently increased the normalised AUC however there was no change between the combination group and control (FIG. 40B). Pre-treatment with WAY100635 prevented the 5-Meo- and cocaine-induced increase in normalised AUC with a significant difference between the cocaine+vehicle and the cocaine+WAY100635 treatment groups (FIG. 40B). The maximum number of intersections were significantly reduced in the WAY100635 treated groups relative to their vehicle treated pairings (FIG. 40C). The average neurite length was significantly increased in the 5-Meo+vehicle treated but not the 5-Meo+WAY100635 group (FIG. 40D).

### Cocaine and 5-MeO-DMT+Ketanserin

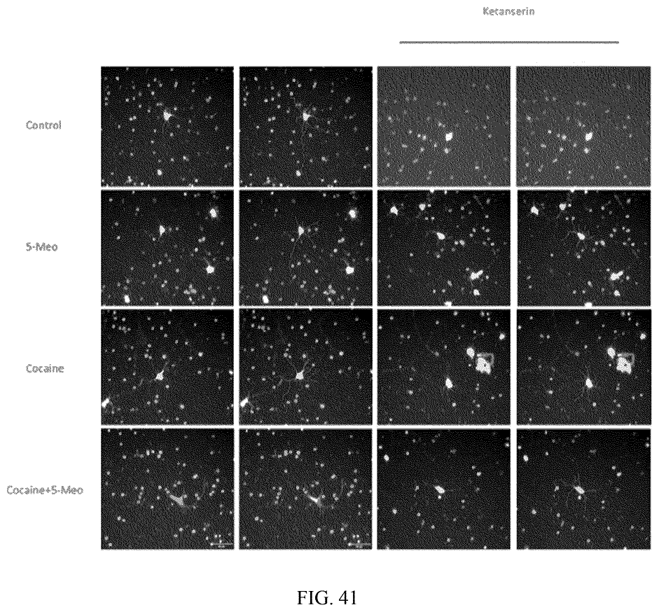

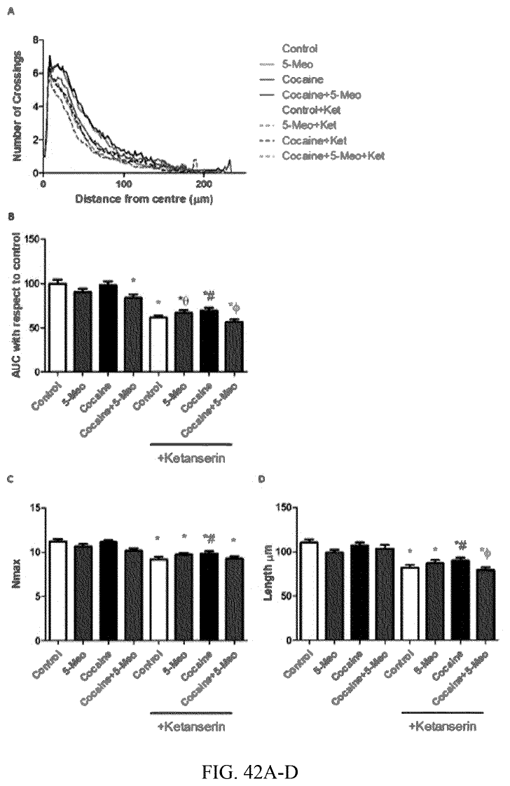

Representative neurons and their corresponding traces for each treatment group are shown in FIG. 41. Sholl plots (FIG. 42A) were generated for the traced neurons (n=130-150/group) in each treatment group by running a Sholl analysis. Ketanserin significantly reduced normalised AUC across all treatment groups (FIG. 42B). The cocaine+5-MeO-DMT combination also reduced AUC relative to control (FIG. 42B). The maximum number of intersections were significantly reduced in all of the ketanserin treated groups relative to control with an additional specific reduction in the cocaine+ketanserin relative to cocaine+vehicle (FIG. 42C). The average neurite length was significantly decreased in all of the ketanserin treated groups relative to control as well as specific differences between cocaine and combination pairings (FIG. 42D).

### Cocaine and 5-MeO-DMT±ANA-12

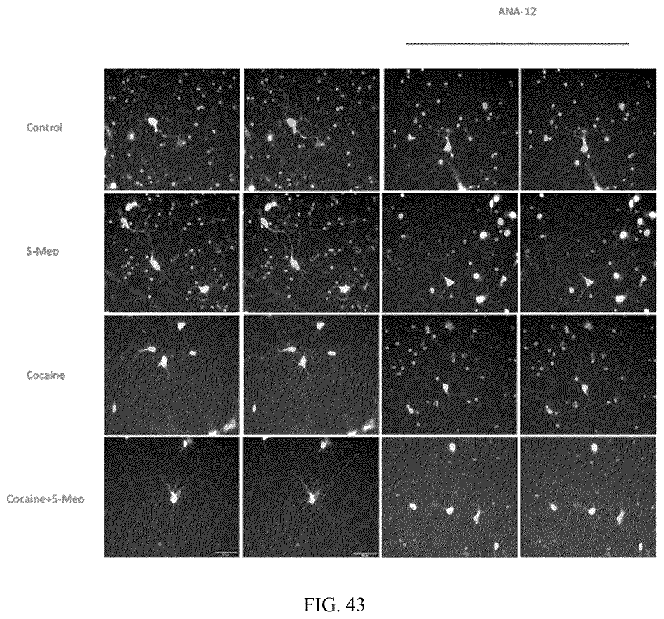

Representative neurons and their corresponding traces for each treatment group are shown in FIG. 43. Sholl plots (FIG. 44A) were generated for the traced neurons (n=129-180/group) in each treatment group by running a Sholl analysis. 5-MeO-DMT and cocaine independently and combined increased the AUC relative to control (FIG. 44B). ANA-12 pre-treatment prevented the 5-Meo-, cocaine- or combination-induced increase in AUC with significant differences between each vehicle and the corresponding ANA-12 pre-treated group (FIG. 44B). There was no difference in AUC between the control+vehicle and control+ANA-12 treatment groups. The maximum number of intersections were significantly reduced in the combination group pre-treated with ANA-12 relative to the vehicle pre-treated combination group, with no other between-group differences present (FIG. 44C). The average neurite length was significantly increased by 5-MeO-DMT alone or in combination with cocaine. This effect was blocked by pre-treatment with ANA-12 (FIG. 44D).

### Cocaine and 5-MeO-DMT±Rapamycin

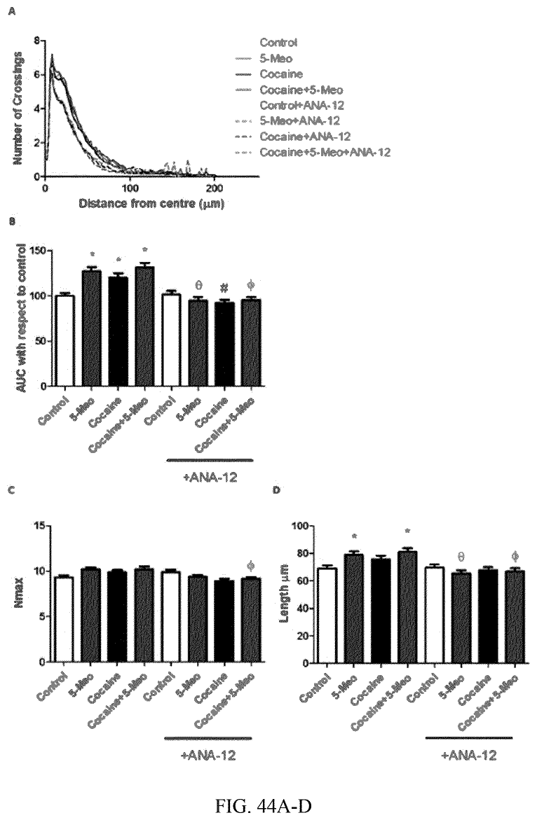

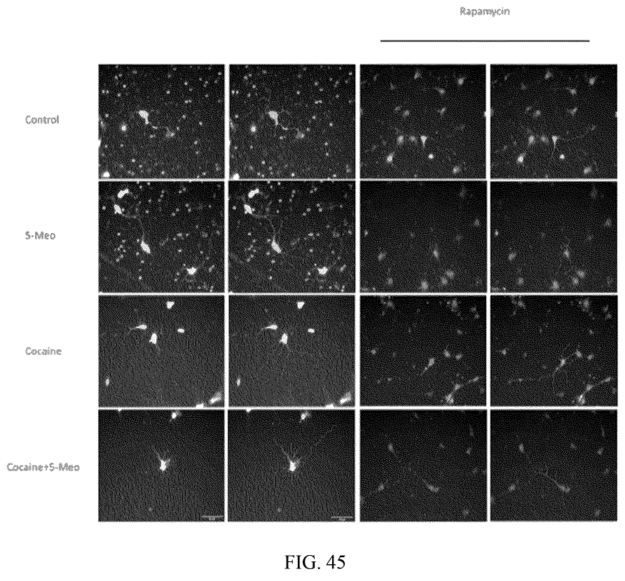

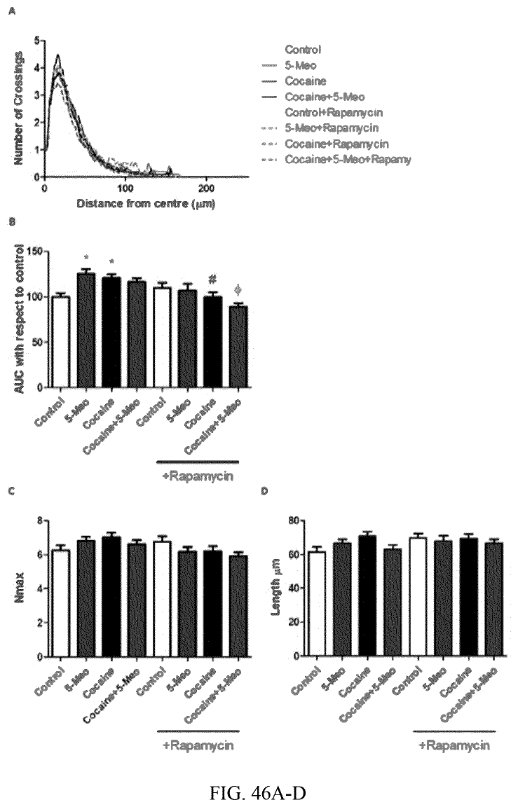

Representative neurons and their corresponding traces for each treatment group are shown in FIG. 45. Sholl plots (FIG. 46A) were generated for the traced neurons (n=90-180/group) in each treatment group by running a Sholl analysis. 5-MeO-DMT and cocaine independently increased the normalised AUC however there was no change between the combination group and control (FIG. 46B). Pre-treating with rapamycin prevented this increase in AUC and lead to specific reductions in the cocaine and combination treatment groups relative to their vehicle pre-treated counterparts (FIG. 46B). The maximum number of intersections and the average neurite length were unchanged between treatment groups (FIG. 46C an FIG. 46D).

### Meta-Analysis of Antagonist Studies

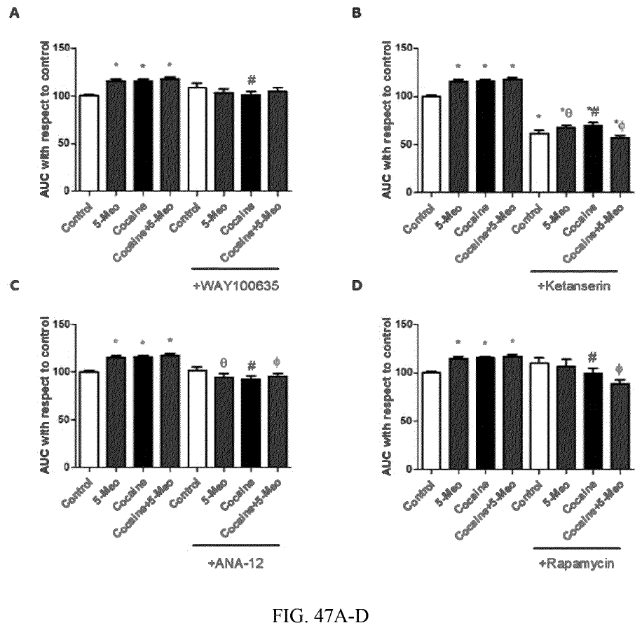

To account for potential variability between independent cultures assessing the same outcome, the vehicle pre-treated control, 5-Meo, cocaine and cocaine+5-MeO-DMT treatment groups from each experiment were combined in a meta-analysis of the antagonist studies. 5-Meo, cocaine and their combination significantly increased neuronal structural complexity as measured by normalised AUC (FIG. 47A-47D). WAY100635 pre-treatment prevented the increase in complexity mediated by all treatments with a specific difference between the cocaine+vehicle and cocaine+WAY100635 groups (FIG. 47A). Ketanserin significantly reduced normalised AUC across all treatment groups (FIG. 47B). ANA-12 prevented the 5-Meo-, cocaine- or combination-induced increase in complexity with significant differences between each vehicle and corresponding ANA-12 treated group (FIG. 47C). Rapamycin prevented the treatment-mediated increase in normalised AUC and lead to specific reductions in the cocaine and combination treatment groups relative to their vehicle pre-treated counterparts (FIG. 47D).

### Statistical Analysis

In all instances the data did not exhibit normal distribution. For analyses involving comparison of three or more groups, the non-parametric Kruskal-Wallis test with Dunn's post hoc test was utilised. No statistics were calculated for the individual points of the Sholl plots. Instead, statistical analyses were performed on the aggregate data i.e., the area under the curve of the Sholl plot.

### Example 9—Chronic Cocaine±5-Meo: Behaviour

Having established 5-MeO-DMT was capable of reversing and normalising a wide array of cocaine-associated molecular changes and demonstrated its ability to promote structural plasticity in vitro, the next aim was to determine whether this molecular adaptation was accompanied by a functional demonstration of 5-Meo-mediated behavioural plasticity. As cocaine withdrawal is known to precipitate heightened anxiety and depression that become primary drivers of relapse in humans, a number of behavioural paradigms measuring internal anxiety and depressive like states in animals were chosen for assessment.

### Open Field

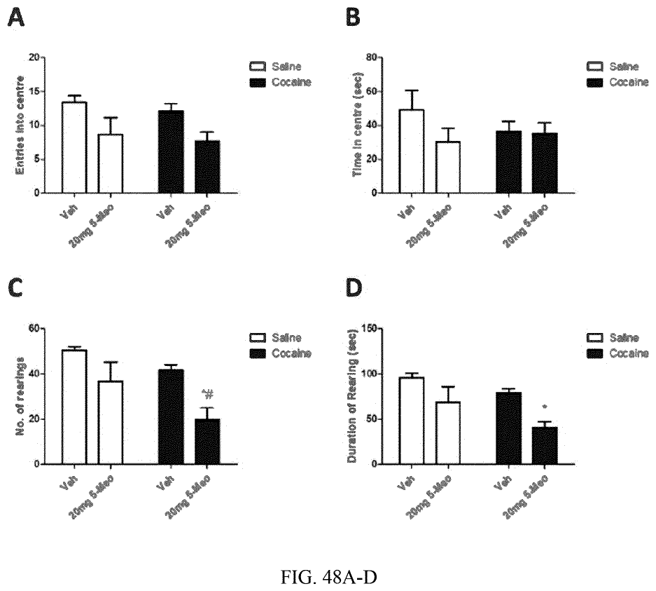

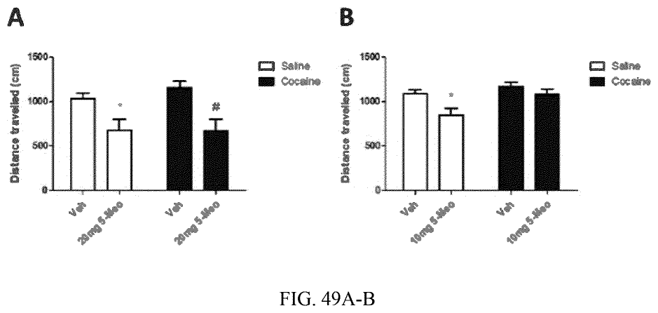

In cohort 1 the number of entries into the centre of the open field arena (FIG. 48A) were significantly affected by 5-MeO-DMT (p=0.0139) with no post hoc differences between treatment groups. The duration of time spent in the centre of the arena (FIG. 48B) was not affected by any treatment group. The number of rearings (FIG. 48C) were significantly affected by both cocaine (p=0.0202) and 5-MeO-DMT (p=0.0020). The combination of cocaine and 20 mg/kg 5-MeO-DMT significantly decreased the number of rearings relative to the control and cocaine-treated animals. The duration of this rearing behaviour was affected by 5-MeO-DMT (p=0.0047) and cocaine (p=0.0380) with a significant decrease in the combination group relative to control (FIG. 48D). There was a significant effect of 5-MeO-DMT on the total distance travelled during the test (p=0.0003) with a reduction in the total distance travelled in the saline+20 mg/kg 5-MeO-DMT and cocaine+20 mg/kg 5-MeO-DMT groups relative to the saline+vehicle and cocaine+vehicle groups respectively (FIG. 49A).

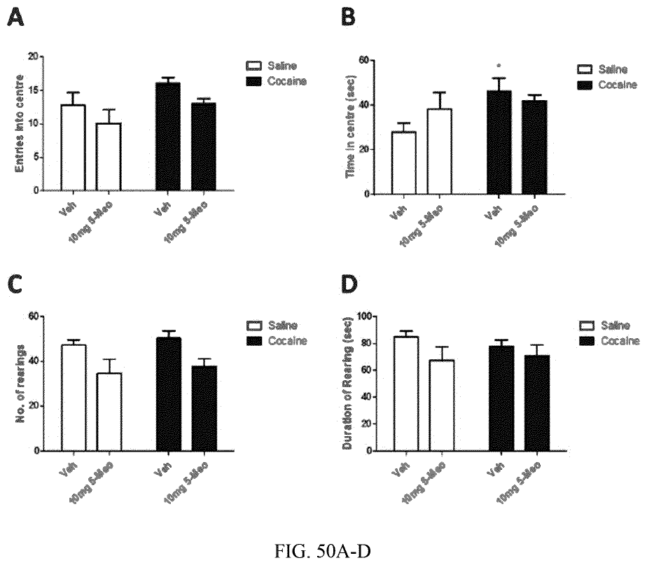

In cohort 2 the number of entries into the centre of the open field were significantly affected by cocaine (p=0.0448) as determined by two-way ANOVA with no post hoc differences between treatment groups (FIG. 50A). The duration of time spent in the centre of the field was not changed by any of treatments (FIG. 50B). There was a significant effect of 5-MeO-DMT on the number (FIG. 50C, p=0.0031) but not the duration of rearings (FIG. 50D, p=0.0837) with no post hoc differences present for either measure. As seen in cohort 1 there was a significant effect of 5-MeO-DMT on the total distance travelled during the test (p=0.0098) with a specific reduction in the total distance travelled in the saline+10 mg/kg 5-MeO-DMT relative to the saline+vehicle group (FIG. 49B). In this cohort cocaine also had a significant effect on the total distance travelled (p=0.0129), but with not specific differences between treatment groups detected by post hoc testing.

### Pre-pulse Inhibition

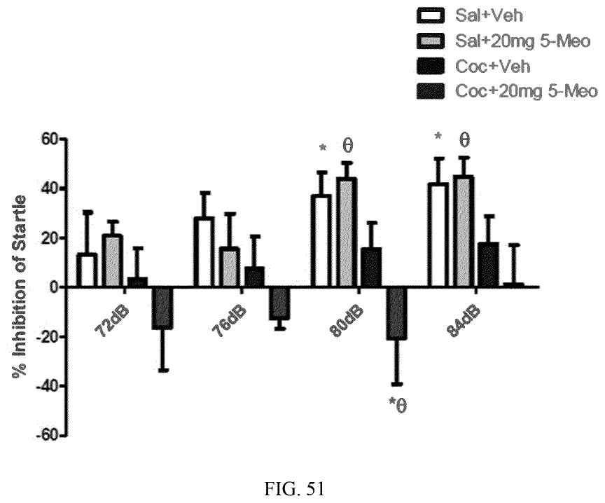

In cohort 1 (FIG. 51) there was a significant effect of both the subjects matching (p<0.0001) and the intensity of the pre-pulse (dB) on the percentage inhibition of the startle response (p=0.0002). There were no treatment effects on PPI. There was a significant increase in the inhibition of the startle response in the control and 5-Meo-treated animals when they received a pre-pulse warning of 80 dB or 84 dB relative to the 72 dB pre-pulse. In the animals treated with cocaine or the combination there was no change in percentage inhibition of startle associated with a change in intensity of the pre-pulse. At the 80 dB pre-pulse there was a significant attenuation of PPI in the combination group relative to both the control and the 5-MeO-DMT alone treatment groups.

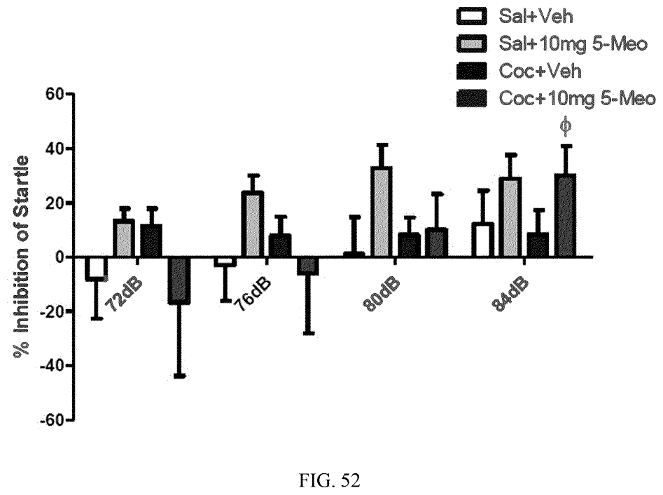

In cohort 2 (FIG. 52) there was a significant effect of both the subjects matching (p<0.0001) and the decibel intensity of the pre-pulse on the percentage inhibition of the startle response (p=0.0011). There was no treatment effect on PPI. There was a significant increase in PPI in the cocaine+10 mg/kg 5-MeO-DMT group when they received a pre-pulse warning of 84 dB relative to their PPI at 72 dB pre-pulse.

### Elevated Plus Maze

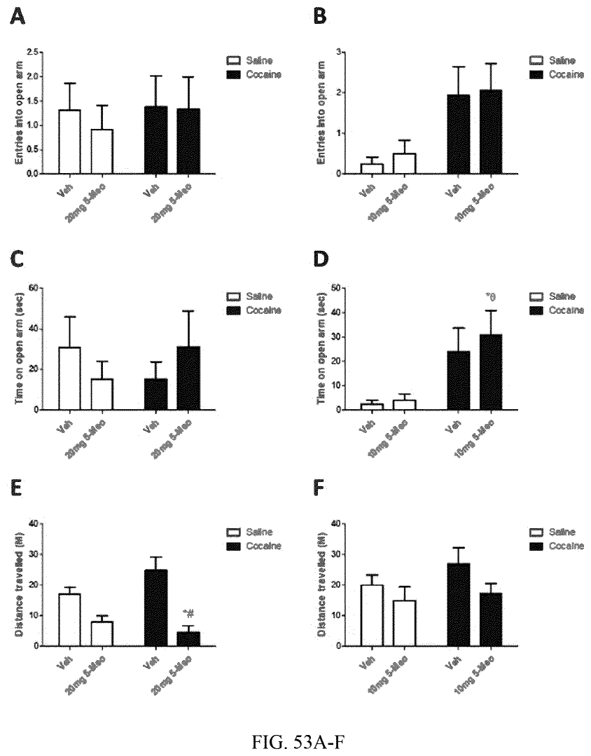

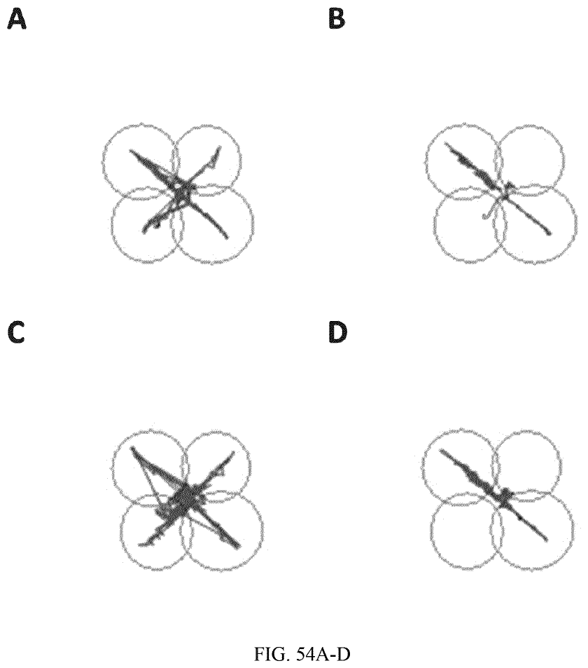

In cohort 1 the number of entries into the open arms (FIG. 53A) and the duration of time spent in the open arms (FIG. 53C) of the maze were unaffected by either treatment or the combination. Similarly, there were no treatment effects on the secondary anxiety measures, stretch-attenuated posture (SAP) or head dipping. A representative trace of an individual animal's movement over the duration of the test for each treatment group is shown in FIG. 54. There was a significant effect of 5-MeO-DMT on the total distance travelled during the test (p<0.0001) with the interaction between cocaine and 5-MeO-DMT approaching significance (p=0.0676). There was a reduction in the total distance travelled in the cocaine+20 mg/kg 5-MeO-DMT combination group relative to the cocaine alone group (FIG. 53E).

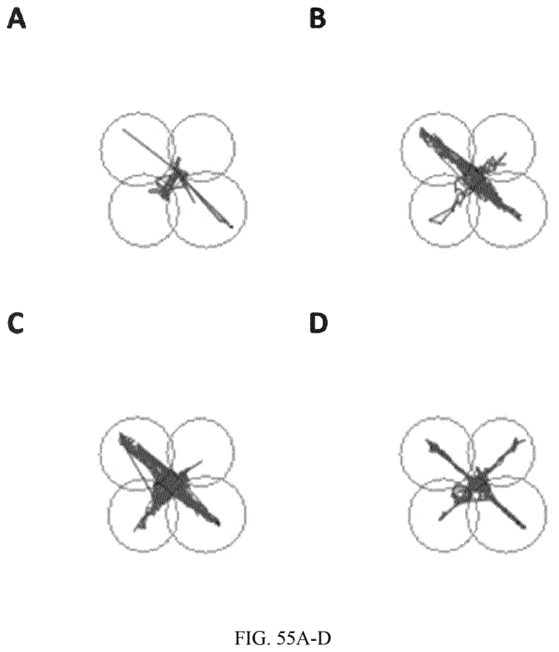

In cohort 2 both the number of entries into the open arms (FIG. 53B, p=0.0055) and the duration of time spent in the open arms of the maze (FIG. 53D, p=0.0031) were significantly affected by cocaine. The duration of time spent on the open arms of the maze was significantly increased in the cocaine and 5-MeO-DMT combination relative to the control and 5-MeO-DMT alone group. The secondary measure of anxiety, head dipping, was significantly affected by cocaine (p=0.0054) however SAP was not (p=0.0502). There were no post hoc differences between treatment groups on either measure. A representative trace of an individual animal's movement over the duration of the test for each treatment group is shown in FIG. 55. There was no effect of either treatment or their combination on the total distance travelled during the test (FIG. 53F).

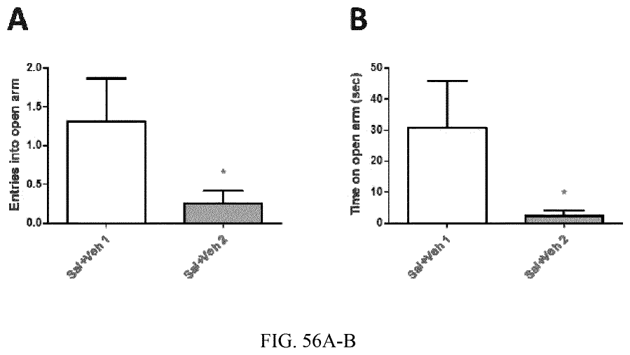

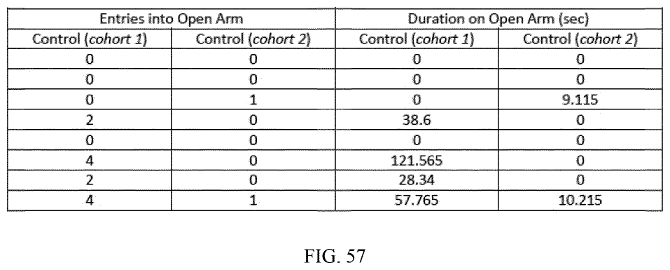

The number of entries into an open arm (FIG. 56A, p=0.0428) and the duration of time spent on the open arms (FIG. 56B, p=0.0417) of the maze were significantly decreased in the control animals of cohort 2 relative to the control animals in cohort 1 (FIG. 57).

### Novel Object Recognition

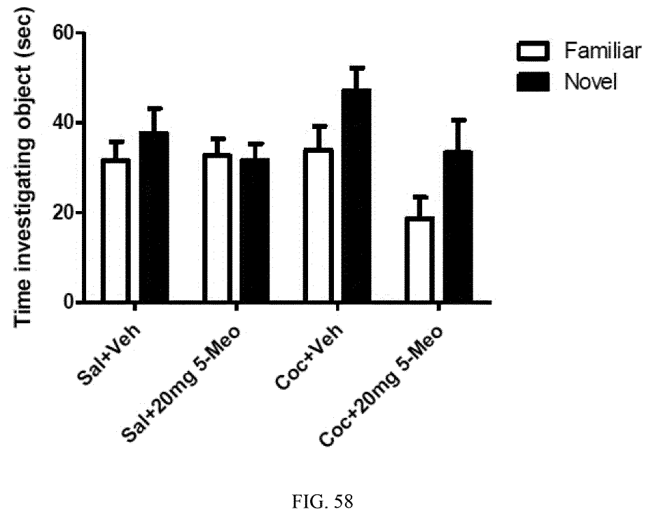

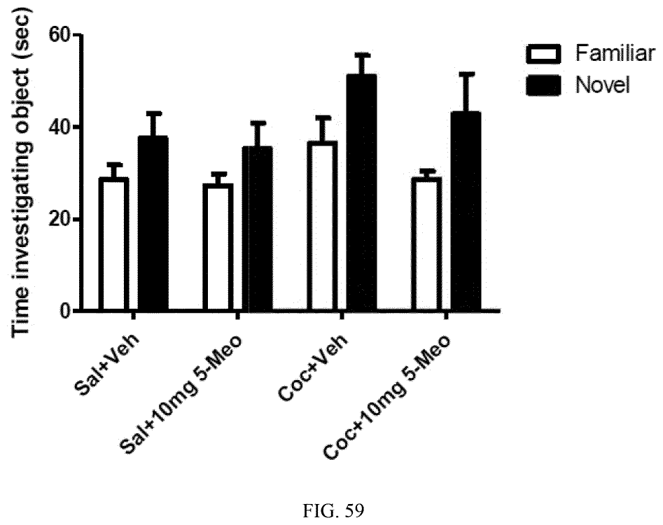

In cohort 1 and cohort 2 the were no differences between treatment groups in time measured investigating the novel object (FIG. 58 and FIG. 59). In both cohorts none of the groups showed a preference for investigating the novel over the familiar object.

### Forced Swim Test

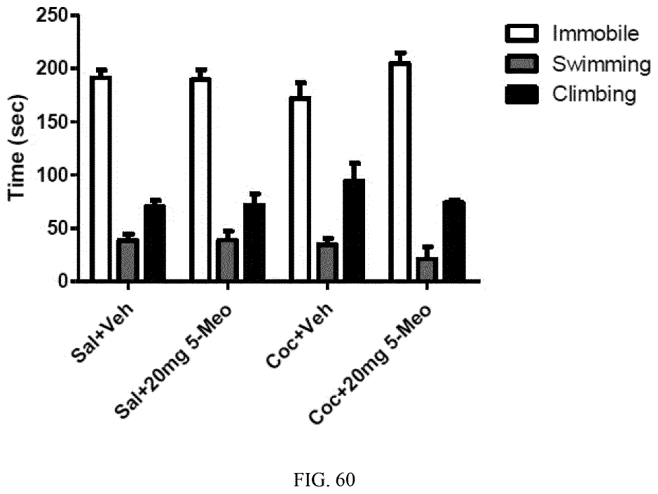

In cohort 1 there were no differences between treatment groups in time spent swimming, climbing or immobile (FIG. 60).

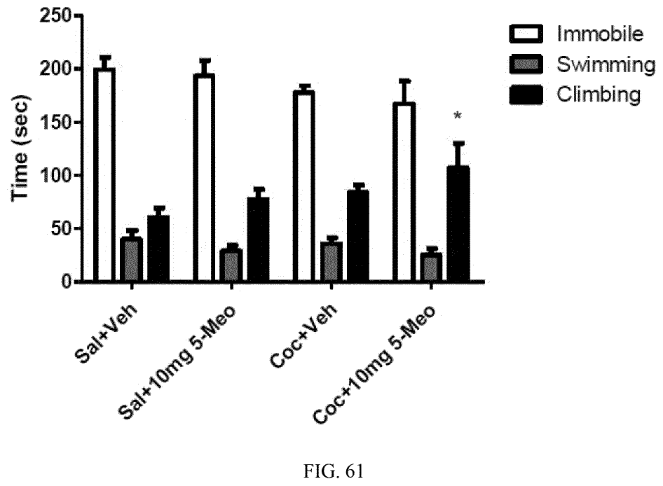

In cohort 2 there was a significant increase in climbing time in the cocaine and 5-MeO-DMT combination group relative to control (FIG. 61). There were no differences in immobility or time spent swimming between treatment groups.

### Example 10—In Vivo Model of Chronic Cocaine with Single Psychedelic Administration

A proteomic characterisation of the action of 5-MeO-DMT in the hippocampus of the Wistar rat model of cocaine addiction was completed to provide a comparison to other psychedelic agents of clinical interest, namely, Lysergic acid diethylamide (LSD), Psilocybin or 2,5-Dimethoxy-4-iodoamphetamine (DOI).

### Method

Male Wister rats received i.p. saline or cocaine (20 mg/kg) daily for 14 days. Animals were then randomly allocated to one of five groups and received a single i.p. injection of one of vehicle, 5 mg/kg 5-Meo-DMT, 0.14 mg/kg LSD, 1 mg/kg psilocybin, or 1 mg/kg DOI. On day 15, animals were euthanised and the hippocampus blunt dissected, snap frozen in liquid nitrogen and stored at −80° C. until further processing.

### Proteomic Analysis

Protein from hippocampal tissue was processed for liquid chromatography tandem Mass Spectrometry (LC-MS/MS). Samples were run on a Thermo Scientific Q Exactive Mass Spectrometer connected to a Dionex Ultimate 3000 (RSLCnano) chromatography system in the UCD Conway Institute Core Mass Spectrometry Facility. Peptide and protein identification of MS/MS spectra were searched against the rat UniProt database using carbamidomethylation (C) as a fixed modification and acetylation (protein N terminus) and oxidation (M) as variable modifications with a false discovery rate filtering of 0.01. MaxQuant was using to generate label-free quantitative (LFQ) ion intensities and the Perseus statistical software contained in the MaxQuant package was used to analyse LFQ ion intensity. Statistical significance (p<0.05, FC±20%) of mean LFQ intensity between treatment groups was determined by two sample t-test. STRING (version 11) was used to generate an association network of the list of proteins identified as being significantly regulated in the NAc or hippocampus by at least one of the psychedelic agents investigated. STRING was also used to generate a subnetwork of proteins identified to be significantly regulated by chronic cocaine exposure, where each node represents a protein and the edges denote a functional association. Networks were visualised using the stringApp (version 1.6.0) plugin for the Cytoscape software platform (version 3.8.2). Signed fold change of difference between saline and cocaine was mapped to nodes using a blue to red gradient. Signed fold change difference of saline v cocaine+psychedelic is visualized as split donut charts around node using a blue to red gradient.

### Biological Pathway Analysis

Subsequent pathway analyses were done using Ingenuity Pathway Analysis (IPA). Analysis settings for IPA included the Ingenuity Knowledge Base (Genes Only), direct and indirect relationships, and endogenous chemicals of the previously filtered list of differentially expressed genes. IPA was filtered to only consider molecules where the species were human, rat, or mouse and the confidence can be experimentally observed. Canonical pathways were utilized in pathway analysis, where the spreadsheet organized the values of significance to −log(p-value) and the rate of regulation to the z-score. Negative z-score values indicate downregulation and positive values indicate upregulation.

### Results

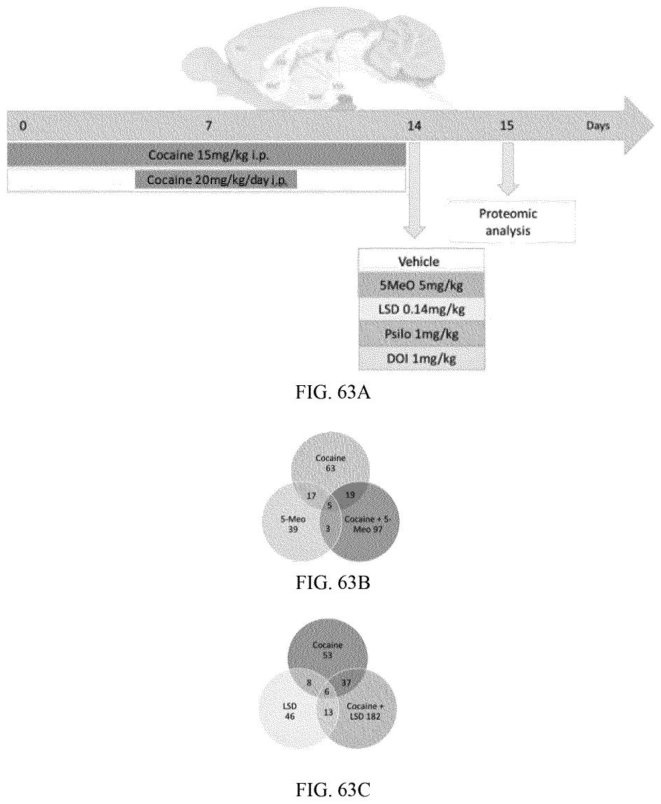

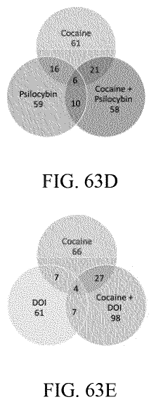

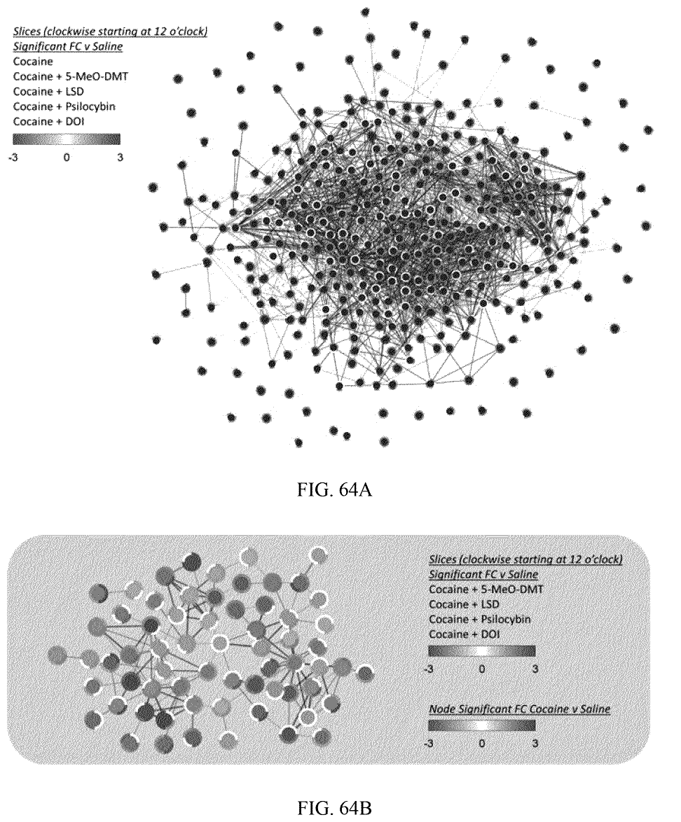

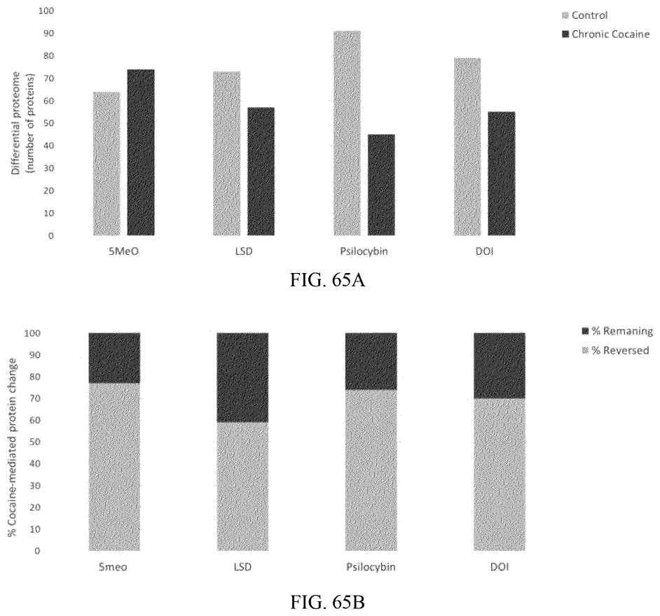

Differential proteomic profile of 5-methoxy-N,N-dimethyltryptamine (5-MeO-DMT), Lysergic acid diethylamide (LSD), Psilocybin or 2,5-Dimethoxy-4-iodoamphetamine (DOI) treatment in hippocampus LC-MS/MS analysis of hippocampus identified 1,459 proteins. Differential expression analysis shows that a single administration of either 5-MeO-DMT (5 mg/kg), LSD (0.14 mg/kg), psilocybin (1 mg/kg) or DOI (1 mg/kg) can significantly alter the proteomic profile of the hippocampus after repeated cocaine administration and in the control brain (FC+/−20%, p<0.05, Student's t-test). Venn diagrams in FIG. 63B-FIG. 63E indicate the overlap in the differential proteomic profile as a result of each psychedelic treatment, by pairwise comparisons with the saline control group. Data are the number of proteins significantly regulated (FC+/−20%, p<0.05, Student's t-test) in each pairwise comparison. There was a total of 373 proteins identified as being significantly regulated in the hippocampus by at least one of the psychedelic agents investigated when compare to saline control (p<0.05, FC+20%). FIG. 64A shows an association network of these 373 proteins. Signed FC difference of saline v cocaine psychedelic is visualised as split donut charts around the node using a blue to red gradient. FIG. 64B shows the STRING network of the 85 significantly regulated proteins in the hippocampus between saline and cocaine (p<0.05, FC: +20%). Signed fold change difference of saline v cocaine was mapped to nodes using a blue to red gradient. Signed fold change difference of saline v cocaine+psychedelic is visualized as split donut charts around node using a blue to red gradient. A single treatment with 5-MeO-DMT resulted in a differential expression of 64 proteins in the control hippocampus and 74 in the chronic cocaine hippocampus. A single treatment with LSD resulted in a differential expression of 73 proteins in the control hippocampus and 57 in the chronic cocaine hippocampus. A single treatment with psilocybin resulted in a differential expression of 91 proteins in the control hippocampus and 45 in the chronic cocaine hippocampus. A single treatment with DOI resulted in a differential expression of 79 proteins in the control hippocampus and 55 in the chronic cocaine hippocampus (FIG. 65A). Data are the number of proteins significantly regulated (p<0.05, FC: +20%, Student's test versus the saline-saline control group or cocaine-saline group) by psychedelic treatment. A single treatment with 5-MeO-DMT, LSD, psilocybin or DOI induced a 77%, 59%, 74% or 70% reversal of chronic cocaine mediated proteomic changes in the hippocampus, respectively (FIG. 65B). Data are expressed as the percentage of significant proteins (p<0.05, FC+/−20%, Student's t-test) reversed to control and remaining altered by cocaine following a single treatment with each psychedelic.

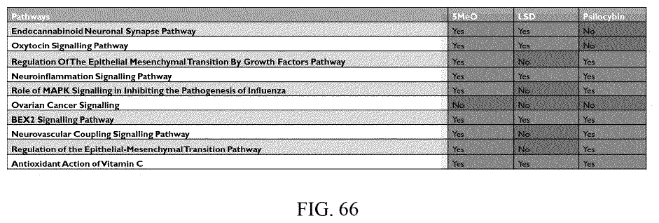

Finally, biological pathway analysis of a corresponding transcriptomics analysis identified pathways dysregulated by cocaine including growth/plasticity, endocannabinoid and oxytocin signalling and neuroinflammation. Looking at the top 10 cocaine-dysregulated pathways, 5-MeO-DMT corrected 90% of these biological pathways, psilocybin 70% and LSD 50% (FIG. 66).

The data highlight the ability of a single treatment of 5-MeO-DMT to reverse cocaine addiction associated molecular changes in the hippocampus. The effect was compared to a single treatment with the psychedelic agents LSD, psilocybin or DOI. Cocaine addiction associated molecular changes were differentially modulated by each psychedelic. Pathway analysis of differentially expressed genes dysregulated by cocaine demonstrates that 5-MeO-DMT corrects 9 of the top 10 dysregulated pathways while psilocybin corrects 7 and LSD corrects 5 of these pathways.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-39. (canceled)

40. A method of treating a substance use disorder in a subject, the method comprising: administering to the subject a therapeutically effective amount of 5-methoxy-N,N-dimethyltryptamine (5-MeO-DMT), or a pharmaceutically acceptable derivative or salt thereof.

41. The method of claim 40, wherein the administration is effective at reducing: self-administration of the substance, relapse, an effect of substance withdrawal, or any combination thereof.

42. The method of claim 40, wherein the substance comprises a stimulant.

43. The method of claim 42, wherein the stimulant is selected from the group consisting of: cocaine, nicotine, methamphetamine, amphetamine, ecstasy, and any combination thereof.

44. The method of claim 42, wherein the stimulant is cocaine.

45. The method of claim 40, wherein the substance comprises a sedative.

46. The method of claim 45, wherein the sedative is selected from the group consisting of a barbiturate, a benzodiazepine, an antihistamine, an antidepressant, an opioid, an antipsychotic, alcohol, and any combination thereof.

47. The method of claim 45, wherein the sedative is heroin.

48. The method of claim 40, wherein the 5-MeO-DMT modulates gene expression of a biomarker present in the subject's blood or urine.

49. The method of claim 40, wherein the 5-MeO-DMT modulates gene expression of a biomarker is selected from the group consisting of: FosB, ΔFosB, cAMP response element binding protein (CREB), histone methyltransferase (G9a), histone H3 lysine 9 (H3K9), metabotropic glutamate receptor (mGluR), glucocorticoid receptor (GR), 5-HT1A Receptor (5-HT1AR), and brain-derived neurotrophic factor (BDNF).

50. The method of claim 49, wherein the biomarker is FosB.

51. The method of claim 49, wherein the biomarker is G9a.

52. The method of claim 49, wherein the biomarker is ΔFosB.

53. The method of claim 49, wherein the biomarker is 5-HT_{1A}R.

54. The method of claim 40, wherein the therapeutically effective amount is from about 1 mg/kg to about 50 mg/kg.

55. The method of claim 40, wherein the therapeutically effective amount is about 10 mg/kg or 20 mg/kg.

56. The method of claim 40, further comprising administering a therapeutically effective amount of an antidote reversal agent selected from the group consisting of: ketanserin, rapamycin, pizotifen, spiperone, ritanserin, WAY100635, and ANA-12.

57. A method of reducing anxiety or depression of a subject suffering from sustained substance exposure, comprising: administering to the subject a therapeutically effective amount of 5-methoxy-N,N-dimethyltryptamine (5-MeO-DMT), or a pharmaceutically acceptable derivative or salt thereof.

58. A method of modulating gene or protein expression of a biomarker in a subject suffering from sustained substance exposure, comprising: administering to the subject a therapeutically effective amount of 5-methoxy-N,N-dimethyltryptamine (5-MeO-DMT), or a pharmaceutically acceptable derivative or salt thereof.

59. A kit, comprising: (a) 5-Methoxy-N,N-dimethyltryptamine (5-MeO-DMT), or pharmaceutically acceptable derivative or salt thereof, and (b) an antidote reversal agent.
